(19) United States
(12) Patent Application Publication
(10) Pub. No.: US 2022/0249413 A1
(43) Pub. Date: Aug. 11, 2022

(54) SWELL 1 MODULATORS FOR TREATMENT OF NON-ALCOHOLIC FATTY LIVER DISEASE, IMMUNE DEFICIENCIES, MALE INFERTILITY AND VASCULAR DISEASES

(71) Applicant: UNIVERSITY OF IOWA RESEARCH FOUNDATION, Iowa City, IA (US)

(72) Inventors: Rajan Sah, Iowa City, IA (US); Susheel Gunasekar, Iowa City, IA (US); Litao Xie, Iowa City, IA (US)

(73) Assignee: UNIVERSITY OF IOWA RESEARCH FOUNDATION, Iowa City, IA (US)

(21) Appl. No.: 17/618,118

(22) PCT Filed: Jun. 10, 2020
(86) PCT No.: PCT/US2020/036992

§ 371 Date: Dec. 10, 2021

Related U.S. Application Data
(60) Provisional application No. 62/859,606, filed on Jun. 10, 2019.

(51) Int. Cl. A61K 31/192; A61K 31/138; A61K 31/40; A61P 1/16

(52) U.S. Cl. CPC A61K 31/192; A61P 1/16; A61K 31/40; A61K 31/138

(57) ABSTRACT

The invention provides the use of SWELL1-LRRC8 modulators and/or binding molecules for therapeutic use, e.g., to treat nonalcoholic fatty liver disease (NAFLD), immune deficiency, and/or male infertility and to regulate vascular tone, systemic arterial and/or pulmonary arterial blood pressure and/or blood flow.

### CROSS-REFERENCE TO RELATED APPLICATION(S)

This application claims priority to U.S. Provisional Application No. 62/859,606 that was filed on Jun. 10, 2019. The entire contents of this application referenced above are hereby incorporated by reference herein.

### BACKGROUND

Nonalcoholic fatty liver disease (NAFLD) is a condition in which fat builds up in the liver. Nonalcoholic steatohepatitis (NASH) is a type of NAFLD. A patient having NASH has inflammation and liver cell damage, along with fat in the liver.

Usually, nonalcoholic fatty liver disease (NAFLD) and nonalcoholic steatohepatitis (NASH) cause few or no symptoms. Doctors use your medical history, a physical exam, and tests to diagnose nonalcoholic fatty liver disease (NAFLD) and nonalcoholic steatohepatitis (NASH). Tests may include blood tests, imaging tests, and sometimes liver biopsy.

Doctors may recommend weight loss to treat nonalcoholic fatty liver disease (NAFLD) and nonalcoholic steatohepatitis (NASH). Weight loss can reduce fat in the liver, inflammation, and fibrosis. However, no medicines have been approved to treat NAFLD and NASH. Accordingly, treatments for NAFLD and NASH are needed.

Normal SWELL1 function is required for normal human immune system development. In one example, expression of a truncated SWELL1 protein caused by a translocation in one allele of SWELL1 inhibits normal B-cell development, causing agammaglobulinemia 5 (AGM5). (Sawada et al., J Clin Invest. 2003 December; 112(11):1707-13) Because different types of immune system cells (e.g. B-lymphocytes and T-lymphocytes) use similar intracellular signaling pathways, it is likely that the development and/or function of other immune system cells (e.g. T-lymphocytes, macrophages, and/or NK cells) would also be affected by inadequate SWELL1 expression or function. Currently, no medicines have been approved to treat agammaglobulinemia 5 and many other deficiencies of immune system cell function.

Currently, the molecular causes of male infertility are only partially understood. In mice lacking SWELL1 late spermatids fail to reduce their cytoplasm during development into spermatozoa and have disorganized mitochondrial sheaths with angulated flagella, resulting in reduced sperm motility. This demonstrates that SWELL1 is also required for normal spermatid development and male fertility. (Luck et al., J Biol Chem. 2018 Jul. 27; 293(30):11796-11808)

### SUMMARY OF CERTAIN EMBODIMENTS

Accordingly, certain embodiments provide a method for preventing and/or treating nonalcoholic fatty liver disease (NAFLD) in a patient in need of such therapy, comprising administering a therapeutically effective amount of a SWELL1 modulator to the patient.

Certain embodiments provide the use of a SWELL1 modulator for preventing and/or treating nonalcoholic fatty liver disease (NAFLD).

Certain embodiments provide a method for regulating vascular tone, systemic arterial and/or pulmonary arterial blood pressure and/or blood flow in a patient in need of such treatment, comprising administering to the patient a therapeutically effective amount of a SWELL1 modulator to the patient.

Certain embodiments provide the use of a SWELL1 modulator for regulating vascular tone, systemic arterial and/or pulmonary arterial blood pressure and/or blood flow.

Certain embodiments provide a method for preventing and/or treating agammaglobulinemia or other immune deficiency in a patient in need of such therapy, comprising administering a therapeutically effective amount of a SWELL1 modulator to the patient.

Certain embodiments provide the use of a SWELL1 modulator for preventing and/or treating agammaglobulinemia or other immune deficiency.

Certain embodiments provide a method for preventing and/or treating male infertility in a patient in need of such therapy, comprising administering a therapeutically effective amount of a SWELL1 modulator to the patient.

Certain embodiments provide the use of a SWELL1 modulator for preventing and/or treating male infertility.

### BRIEF DESCRIPTION OF THE FIGURES

FIGS. 1a-1g. I_{Cl,SWELL} and SWELL1 protein are reduced in T2D β-cells and adipocytes. a-b. Current-voltage plots of I_{Cl,SWELL} measured in non-T2D and T2D mouse (a) and human (b) β-cells at baseline (iso, black trace) and; with hypotonic stimulation (hypo, red trace). c-d. Mean inward and outward I_{Cl,SWELL} current densities at +100 and −100 mV from non-T2D (n=3 cells) and T2D (n=6 cells) mouse (c) and non-T2D (n=6 cells) and T2D (n=22 cells) human (d) β-cells. e. Mean inward and outward I_{Cl,SWELL} current densities at +100 and −100 mV from adipocytes isolated from visceral fat of lean^{#} (n=7 cells), obese non-T2D^{#} (n=13 cells) and T2D patients (n=5 cells). f. Western blot detecting SWELL1 protein expression in inguinal adipose tissue isolated from a polygenic-T2D KKA^{y} mouse (12 months old) compared to its control strain KKA^{a} (12 months old) and wild-type C57BL/6 mouse (14 months old) respectively. g. Western blot comparing SWELL1 protein expression in visceral adipose tissue isolated from lean, obese non-T2D and obese T2D patients respectively. #Data from lean and obese non-T2D adipocytes replotted from our previously reported data in Zhang, Y et al. (2017) for purposes of comparison. Data are represented as mean±SEM. Two-tailed unpaired t-test was used in c and d. Two-tailed permutation t-test group comparison was used in e. * and ** represents p<0.05 and p<0.01 respectively.

FIGS. 2a-2k. SWELL1 protein expression regulates insulin stimulated PI3K-AKT2-AS160 signaling. a. Western blots detecting levels of SWELL1, pAKT2, AKT2 and β-actin with 0 and 10 nM insulin stimulation for 15 min in wildtype (WT, black), SWELL1 knockout (KO, red) and adenoviral overexpression of SWELL1 in KO (KO+SWELL1 O/E, blue) 3T3-F442A adipocytes (left). The corresponding densitometric ratio for pAKT2/β-actin are shown to the right (n=3 independent experiments for each condition). b. Mean inward and outward current densities at +100 and −100 mV from WT (black, n=5 cells), KO (red, n=4 cells) and KO+SWELL1 O/E (blue, n=4 cells) 3T3-F442A preadipocytes. c-d. Western blots comparing levels of SWELL1, pAKT2, AKT2 and 3-actin (c) and pAS160, AS160 and 3-actin (d) with 0 and 10 nM insulin stimulation in wildtype (WT, black) and SWELL1 overexpression in WT (WT+SWELL1 O/E, blue) 3T3-F442A adipocytes (n=6 independent experiments for each condition). The corresponding densitometric ratio for pAKT2/β-actin and total AKT2 are shown to the right in (c) and pAS160/β-actin (right top) and total AS160 (right bottom) in (d). e) Cartoon model of homomeric mouse LRRC8a/SWELL1 derived from cryo-electron microscopy (EM) and x-ray crystallography structure (PDB ID: 6G90^{#}) and the inset (shown as dimer for descriptive purpose) showing Smod1/DCPIB bound in the pore region derived from DCPIB bound SWELL1 cryo-EM structure (PDB ID: 6NZW^{$)} (bottom) and chemical structure of Smod1/DCPIB (top). f) I_{Cl,SWELL} inward and outward current over time upon hypotonic stimulation and subsequent inhibition by 10 μM Smod1 in BEK cells. g) Western blots detecting levels of SWELL1, pAKT2 and β-actin with 0, 3 and 10 nM insulin-stimulation in WT 3T3-F442A preadipocytes (n=2 independent experiments for each condition, top) and corresponding densitometric ratio for SWELL1/β-actin and pAKT2/β-actin (bottom); h) SWELL1, pAKT2, AKT2 and R-actin with 0 and 10 nM insulin in WT and KO 3T3-F442A adipocytes (n=6 independent experiments for each condition) and the corresponding densitometric ratio for SWELL1/β-actin (i) and pAKT2/β-actin (top) and pAKT2/AKT2 (bottom) (j), respectively; k) pAS160, AS160 and j-actin with 0 and 10 nM insulin in WT 3T3-F442A adipocytes (n=3 independent experiments for each condition, left) and the corresponding densitometric ratio of pAS160/AS160 (right) incubated in either vehicle or Smod1 for 96 h. All densitometries are normalized to values of 0 nM insulin of WT 3T3-F442A pre-/adipocytes except for bottom panel j) where the pAKT2/AKT2 normalization was done to 0 nM insulin for WT and 0 nM insulin for KO values respectively due to the differential expression of total AKT2 in WT and KO. ^{#}Deneka et al. (2018) and ^{$}Kern et al. (2019). Data are represented as mean±SEM. Two-tailed unpaired t-test was used in a-d, g and i-k where *, ** and *** represents p<0.05, p<0.01 and p<0.001 respectively.

FIGS. 3a-3m. Smod1 induces SWELL1 and improves systemic glucose homeostasis in murine T2D models by enhancing insulin sensitivity and secretion a. Western blots detecting SWELL1 protein expression in visceral fat of C57BL/6 mice on high-fat diet (HFD) for 21 weeks treated with either vehicle or Smod1 (5 mg/kg i.p.×3-6 days, as described in scheme FIG. 4e) and the corresponding densitometric ratio for SWELL1/β-actin (right) (n=6 mice in each group). b. Western blots comparing SWELL1 protein expression in inguinal adipose tissue of a polygenic-T2D KKA^{y} mouse treated with Smod1 (5 mg/kg i.p daily×14 days) compared to untreated control KKA^{a} and wild-type C57/B6 mice. c. Glucose tolerance test (GTT) and insulin tolerance test (ITT) of C57BL/6 mice on HFD for 8 weeks treated with either vehicle or Smod1 (5 mg/kg i.p) for 10 days (n=7 mice in each group). d-f. Fasting glucose levels (d), GTT (e) and ITT (f) of polygenic-T2D KKA^{y} mice (n=6) compared to its control strain KKA^{a} (n=3) pre- and post-Smod1 (5 mg/kg i.p) treatment for 4 days. g-h. Fasting glucose levels (g) of regular chow-diet fed, lean mice treated with either vehicle or Smod1 (5 mg/kg i.p) for 6 days and the corresponding GTT (h) (n=6 in each group). i-j. Fasting glucose levels (i) and GTT (16 weeks HFD, 4 days treatment) and ITT (18 weeks HFD, 4 days treatment) (j) of HFD-T2D mice treated with either vehicle or Smod1 (5 mg/kg i.p). k. Relative insulin secretion in plasma of HFD-T2D mice (18 weeks HFD, 4 days treatment) after i.p. glucose (0.75 g/kg BW) treated with either vehicle (n=3) or Smod1 (n=4, 5 mg/kg i.p). 1-m. Glucose stimulated insulin secretion (GSIS) perifusion assay from islets isolated from HFD-T2D mouse (21 week timepoint) treated with either vehicle (n=3 mice, and 3 experimental replicates) or Smod1 (n=3 mice, and 2 experimental replicates, 5 mg/kg i.p) (1) and from polygenic-T2D KKA^{y} mouse treated with either vehicle or Smod1 (5 mg/kg i.p for 6 days, n=3 mice in each group, 3 experimental replicates), (m); and their corresponding area under the curve (AUC) comparisons respectively on the right. Data are represented as mean±SEM. Two-tailed unpaired t-test was used in a, g, i, l and m. Paired t-test was used in d. Paired (in group) and unpaired (between group) t-tests performed in k. Two-way ANOVA was used for c, e, f, h and j. Statistical significance is denoted by *, ** and *** representing p<0.05, p<0.01 and p<0.001 respectively.

FIGS. 4a-4i. Smod1 improves systemic insulin sensitivity, tissue glucose uptake and non-alcoholic fatty liver disease in murine T2D models. a. Mean glucose-infusion rate during euglycemic hyperinsulinemic clamps of KKA^{y} mice treated with vehicle (n=7) or Smod1 (n=8) for 4 days. b. Hepatic glucose production at baseline and during euglycemic hyperinsulinemic clamp of KKA^{y} mice treated with vehicle or Smod1 (n=9 in each group). c. Glucose uptake determined from 2-DG uptake in adipose (iWAT, gWAT) and heart during traced clamp of KKA^{y} mice treated with vehicle or Smod1 (n=9 in each group). d. Glucose uptake into glycogen determined from 2-DG uptake in liver (n=9 for vehicle and n=8 for Smod1), adipose (iWAT, n=7 vehicle and n=6 Smod1) and gastrocnemius muscle (n=7 vehicle and n=6 Smod1) during clamp of KKA^{y} mice. e. Schematic representation of treatment protocol of C57BL/6 mice injected with either vehicle or Smod1 (n=6 in each group) during HFD-feeding. f. Liver mass (left) and normalized ratio to body mass (right) of HFD-T2D mice following chronic-intermittent treatments of either vehicle or Smod1 (5 mg/kg i.p.). g-i. Corresponding hematoxylin-eosin stained liver sections (g), liver triglycerides (6 mice in each group) (h), and the corresponding NAFLD-activity score (NAS) based on hepatocyte steatosis, inflammation and ballooning (i). Data are represented as mean±SEM. Two-tailed unpaired t-test was used in a-d, f, h and i. Statistical significance is denoted by *, ** and *** representing p<0.05, p<0.01 and p<0.001 respectively. Scale bar—100 μm.

FIGS. 5A-5C. Adipocyte SWELL1 deletion (Adipo KO) limits adiposity in the setting of obesity under high-fat high-sucrose diet (58% kcal fat, 18% sucrose, 27 weeks) and exacerbates non-alcoholic fatty liver disease (NAFLD). a) Representative images of epididymal (eWAT) and inguinal (iWAT) fat pads (left). Total fat pad weights and ratio of fat pad over body weight of WT (n=10) and Adipo KO (n=8) mice fed with high-fat/high-sucrose (HFHS) diet for 27 weeks (right). b) Representative images of liver tissue dissected from WT (top) and Adipo KO (bottom). Corresponding total liver mass and ratio of liver mass over body weight. c) Representative images of haematoxylin and eosin (H&E) stained liver sections of WT and Adipo KO mice and liver steatosis estimated from H and E sections. Scale bar: 100 μm.

FIGS. 6A-6C. Adipocyte SWELL1 deletion (Adipo KO) limits adiposity in short-term high fat diet (60% kcal fat, 8 weeks) and predisposes to developing non-alcoholic fatty liver disease (NAFLD). a) Total body weight of WT and Adipo KO (n=7 each group) mice fed with high-fat diet for 8 weeks. b) Total mass of dissected epididymal (eWAT) and inguinal (iWAT) fat pads and their corresponding total fat pad weights and ratio of fat pad over body weight. c) Total liver mass and ratio of liver mass over body weight dissected from WT and Adipo KO mice respectively.

FIGS. 7A-7C. Adipocyte SWELL1 deletion (Adipo KO) limits adiposity in long-term high fat diet (60% kcal fat, 19 weeks) and predisposes to developing non-alcoholic fatty liver disease (NAFLD). a) Total body weight of WT (n=7) and Adipo KO (n=6) mice fed with high-fat diet for 19 weeks. b) Total mass of dissected epididymal (eWAT) and inguinal (iWAT) fat pads and their corresponding total fat pad weights and ratio of fat pad over body weight. c) Total liver mass and ratio of liver mass over body weight dissected from WT and Adipo KO mice respectively.

FIG. 8. SWELL1 protein is reduced in ageing. Representative image of western blot comparing SWELL1 protein expression in epididymal adipose tissue isolated from young (2 months old, females) and aged (18 months old, females) C57BL/6 mice fed with regular-chow diet respectively.

FIGS. 9a-9d. Adipocyte SWELL1 deletion (Adipo KO) limits adiposity with aging (regular-chow diet, 18 months old) and predisposes to non-alcoholic fatty liver disease (NAFLD). a) Total body weight of WT (n=15) and Adipo KO (n=11) mice fed with regular chow diet for 18 months. b) Total mass of dissected epididymal (eWAT) and inguinal (iWAT) fat pads and their corresponding total fat pad weights and ratio of fat pad over body weight. c) Total liver mass and ratio of liver mass over body weight dissected from WT and Adipo KO mice respectively. d) Representative images of haematoxylin and eosin (H&E) stained liver sections of WT and Adipo KO mice and their corresponding liver steatosis estimated from H and E sections. Scale bar: 100 μm.

FIGS. 10A-10E. FIG. 10 depicts results demonstrating that SWELL1 is required for prominent VRAC currents in human umbilical vein endothelial cells. FIG. 10A, Western blot of endogenous SWELL1 in Ad-shSCR and Ad-shSWELL1 transduced HUVECs. FIG. 10B, SWELL1 immunoflurorescence staining. FIG. 10C, SWELL1 mediated VRAC in response to voltage-steps from −100 to +100 mV. FIG. 10D, Current-voltage relationships in shSCR and shSWELL1 transduced HUVECs. FIG. 10E, Mean current densities at −100 mV and +100 mV.

FIGS. 11A-11B. FIG. 11 depicts results demonstrating that SWELL1 is necessary for insulin-PI3K, ERK signaling in endothelium.

FIGS. 12A-12K. FIG. 12 depicts results of time-course experiments over 6 hours of insulin-stimulation.

FIGS. 13A-13K. FIG. 13 depicts results demonstrating that overexpressing SWELL1 above endogenous SWELL1 protein levels is sufficient to augment insulin-stimulated pAKT, eNOS, p-eNOS and pERK (MAP kinase signaling) in HUVECs, while reducing p-p70, pS6 ribosomal protein (mTOR signaling).

FIGS. 14A-14B. FIG. 14 depicts results suggesting that SWELL1 resides in a signaling complex that includes GRB2, Cav1 and eNOS.

FIGS. 15A-15B. FIG. 15 depicts results indicating that SWELL1 regulates stretch-dependent AKT and ERK1/2 signaling in HUVECs.

FIGS. 16A-16C. FIG. 16 depicts results that SWELL1 KD HUVECs are significantly 4-fold larger (FIG. 7A) and migrate >5-fold faster in scratch assays (FIG. 7B) compared to controls. Conversely, SWELL1 overexpression significantly slows migration rate to control levels (FIG. 7C).

FIGS. 17A-17B. FIG. 17 depicts results that indicate that SWELL1 may regulate angiogenesis via mTOR signaling.

FIG. 18. FIG. 18 depicts results that genome-wide transcriptome analysis of SWELL1 KD HUVEC compared to control (RNA sequencing) reveal multiple pathways enriched regulating angiogenesis, migration and tumorigenesis, including GADD45, IL-8, p70S6K, TREM1, angiopoeitin and HGF signaling.

FIG. 19. FIG. 19 depicts results consistent with endothelial dysfunction and impaired vascular relaxation in eSWELL1 KO mice, resulting in a propensity for systolic hypertension.

FIG. 20. FIG. 20 depicts results that, in mice raised on HFHS diet, retinal blood flow is more severely impaired with significant focal, and diffuse retinal vessel narrowing in eSWELL1 KO mice compared to WT mice, and this is markedly worse in female compared to male mice. It has also been demonstrated that SWELL1 is both necessary and sufficient for insulin-AKT, eNOS, ERK and mTOR signaling in endothelium, and that this signaling is independent of SWELL1-mediated plasma membrane VRAC activity.

FIGS. 21a-21e. Transient expression of full-length SWELL1 with C-terminal 3λFlagtag rescues ICl,SWELL and traffics to the plasma membrane. a-c. Current-voltage plots of ICl,SWELL measured in 3T3-F442A preadipocytes WT (a), KO (b) and adenoviral overexpression of SWELL1 in KO (KO+SWELL1 O/E) (c) at baseline (iso, black trace) and hypotonic (hypo, red trace) stimulation respectively. d. Immunostaining images demonstrating localization of endogenous SWELL1 or overexpressed SWELL1 with anti-Flag or anti-SWELL1 antibody (Scale bar—20 μm). e. Validation of SWELL1 antibody in WT 3T3-F442A compared to SWELL1 KO pre-adipocytes (Scale bar—20 μm), revealing a punctate pattern of endogenous SWELL1 localization (inset).

FIGS. 22a-22d. Smod1 does not induce hypoglycemia in lean, non-T2D mice and maintains normoglycemia in murine T2D with chronic treatment without overt signs of toxicity. Fasting glucose levels (a), GTT (b) and ITT (c) of C57BL/6 lean mice on regular chow diet treated with either vehicle or Smod1 (5 mg/kg i.p) for 10 days (n=7 males in each group). d. GTT of HFD-T2D mice (8 weeks HFD) treated with either vehicle (n=5 males) or Smod1 (5 mg/kg i.p, n=4 males) for 8 weeks. Data are represented as mean±SEM. Two-tailed unpaired t-test was used in a. Two-way ANOVA was used for b-d. Statistical significance is denoted by *, ** and *** representing p<0.05, p<0.01 and p<0.001 respectively and ‘ns’ indicates the difference was not significant.

FIG. 23. Smod1 does not induce hyperglycemia upon acute treatment. Fasting glucose levels of 1-month HFD fed C57BL/6 mice after fasting for 6 h (0 min) and 30 min post-injection of either vehicle or Smod1 (5 mg/kg i.p) (n=8 males in each group). Data are represented as mean±SEM. Two-tailed unpaired t-test was used for the measurement. Statistical significance is denoted by *, ** and *** representing p<0.05, p<0.01 and p<0.001 respectively and ‘ns’ indicates the difference was not significant.

FIG. 24. Smod1 does not affect glucose uptake in brown fat and skeletal muscle. Glucose uptake determined from 2-DG uptake in brown fat, extensor digitorum longus (EDL), soleus and gastrocnemius muscles harvested under clamp for KKAy mice treated with vehicle or Smod1 (n=9 in each group, 5 mg/kg i.p) for 4 days. Data are represented as mean±SEM. Two-tailed unpaired t-test was used for the analysis. ‘ns’ indicates the difference was not significant.

FIG. 25. Smod1 improves non-alcoholic fatty liver disease in murine T2D models. Images of hematoxylin and eosin stained liver histology sections of HFD-T2D mice treated with either vehicle or Smod1 (5 mg/kg i.p) as in FIG. 4e. Scale—(10×: 100 m and 20×: 50 μm).

### DETAILED DESCRIPTION

Accordingly, certain embodiments provide a method for preventing and/or treating nonalcoholic fatty liver disease (NAFLD) in a patient in need of such therapy, comprising administering a therapeutically effective amount of a SWELL1 modulator to the patient.

Certain embodiments provide the use of a SWELL1 modulator for preventing and/or treating nonalcoholic fatty liver disease (NAFLD).

Certain embodiments provide a method for regulating vascular tone, systemic arterial and/or pulmonary arterial blood pressure and/or blood flow in a patient in need of such treatment, comprising administering to the patient a therapeutically effective amount of a SWELL1 modulator to the patient.

In certain embodiments, the method comprises administering to the patient a therapeutically effective amount of a SWELL1 modulator to the patient so as to regulate vascular tone.

In certain embodiments, the method comprises administering to the patient a therapeutically effective amount of a SWELL1 modulator to the patient so as to regulate systemic arterial and/or pulmonary arterial blood pressure.

In certain embodiments, the method comprises administering to the patient a therapeutically effective amount of a SWELL1 modulator to the patient so as to regulate blood flow.

Certain embodiments provide the use of a SWELL1 modulator for regulating vascular tone, systemic arterial and/or pulmonary arterial blood pressure and/or blood flow.

Certain embodiments provide a method for preventing and/or treating agammaglobulinemia or other immune deficiency in a patient in need of such therapy, comprising administering a therapeutically effective amount of a SWELL1 modulator to the patient.

Certain embodiments provide the use of a SWELL1 modulator for preventing and/or treating agammaglobulinemia or other immune deficiency.

Certain embodiments provide a method for preventing and/or treating male infertility in a patient in need of such therapy, comprising administering a therapeutically effective amount of a SWELL1 modulator to the patient.

Certain embodiments provide the use of a SWELL1 modulator for preventing and/or treating male infertility.

In certain embodiments, the SWELL1 modulator is DCPIB, clomiphene, nafoxidine or tamoxifen.

In certain embodiments, the SWELL1 modulator is DCPIB.

In certain embodiments, the SWELL1 modulator is a compound of formula I, or a salt thereof

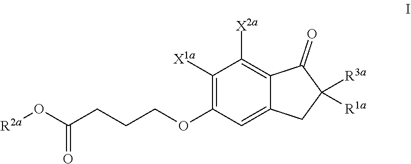

wherein:

X^{1a} and X^{2a} are independently halo;

R^{1a} is C₁₋₆ alkyl, 3-6 membered cycloalkyl, or phenyl, wherein the C₁₋₆ alkyl is optionally substituted with 3-6 membered cycloalkyl;

R^{2a} is hydrogen or C₁₋₆ alkyl, wherein the C₁₋₆ alkyl is optionally substituted with carboxy; and

R^{3a} is C₁₋₆ alkyl.

In certain embodiments, the SWELL1 modulator is a compound of formula II, or a salt thereof

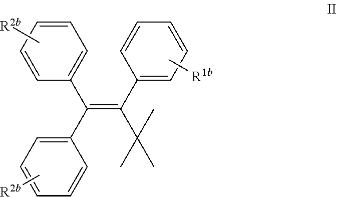

wherein:
R^{1b} is hydrogen, halo or methoxy;
only one of R^{2b} is —O(CH₂)ₙ—NR^{3b}R^{4b};
the other R^{2b} is hydrogen, halo or methoxy;
each of R^{3b} and R^{4b} is independently H or C₁₋₆ alkyl, or R^{3b} and R^{4b} together with the nitrogen to which they are attached form aziridino, azetidino, morpholino, piperazino, pyrrolidino or piperidino;

n is an integer from 2 to 4; and

X is halo.

In certain embodiments, the SWELL1 modulator is a compound of formula III, or a salt thereof

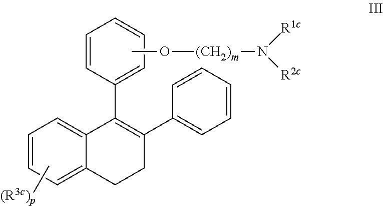

wherein:

each of R^{1c} and R^{2c} is independently H or C₁₋₈ alkyl, or R^{1c} and R^{2c} together with the nitrogen to which they are attached form aziridino, azetidino, morpholino, piperazino, pyrrolidino or piperidino; wherein the aziridino, azetidino, morpholino, piperazino, pyrrolidino and piperidino are optionally substituted with one or more C₁₋₆ alkyl;

m is an integer from 2 to 6,

R^{3c} is C₁₋₈ alkoxy; and

p is an integer from 1 to 4.

In certain embodiments, the SWELL1 modulator is a compound of formula IV, or a salt thereof

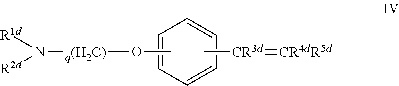

wherein:

each of R^{1d} and R^{2d} is independently H or C₁₋₆ alkyl, or R^{1d} and R^{2d} together with the nitrogen to which they are attached form aziridino, azetidino, morpholino, piperazino, pyrrolidino or piperidino;

each of R^{3d} and R^{4d} is independently aryl which is optional substituted with one or more groups selected from C₁₋₆ alkyl, C₁₋₆ alkoxy, C₁₋₆ dialkylamino, or halo;

R^{5d} is C₁₋₆ alkyl or C₁₋₆ alkenyl, wherein the C₁₋₆ alkyl is optionally substituted with aryl; and

q is an integer from 2 to 6.

In certain embodiments, the administration or use of the SWELL1 modulator and/or SWELL1-LRRC8 binding molecule is sufficient to upregulate the expression of SWELL1 and/or stability and/or assembly of SWELL1-LRRC8 complexes and/or membrane trafficking and/or SWELL1-LRRC8 signaling, or alter expression and/or associated of a SWELL1 associated protein (e.g., LRRC8b,c,d,e, GRB2, Cav1, IRS1, or IRS2).

In certain embodiments, the administration or use of the SWELL1 modulator is sufficient to upregulate the expression of SWELL1.

In certain embodiments, the SWELL1-LRRC8 modulator is DCPIB, clomiphene, nafoxidine or tamoxifen.

In certain embodiments, the SWELL1-LRRC8 modulator is a SWELL1 inhibitor.

In certain embodiments, the SWELL1-LRRC8 modulator is a SWELL1 activator.

In certain embodiments, the SWELL1-LRRC8 modulator is a SWELL1 binding molecule or protein

In certain embodiments, the SWELL1-LRRC8 modulator stabilizes SWELL1-LRRC8 assembly and trafficking to the plasma membrane

In certain embodiments, the SWELL1-LRRC8 modulator augments SWELL1-LRRC8 signaling.

In certain embodiments, the SWELL1-LRRC8 modulator is DCPIB.

In certain embodiments, the administration or use of the SWELL1 modulator is sufficient to upregulate the expression of SWELL1 protein.

In certain embodiments, the SWELL1-LRRC8 modulator or modulator is DCPIB, clomiphene, nafoxidine or tamoxifen or a compound that modulates SWELL1-LRRC8 activity or expression levels. In certain embodiments, the compound is a compound of formula I, II, III or IV, or a salt thereof.

In certain embodiments, the administration or use of the SWELL1 modulator is sufficient to upregulate the expression, and/or assembly and/or trafficking of SWELL1, and/or accessory proteins, including but not limited to LRRC8b, LRRC8c, LRRC8d, LRRC8e, GRB2, Cav1, IRS1, IRS2.

In certain embodiments, the SWELL1-LRRC8 modulator is a compound of formula I, II, III, or IV, or a salt thereof.

In certain embodiments, the modulator alters pannexin channel activity, expression or function, as pannexin proteins are homologous to SWELL1/LRRC8 proteins.

Healthy expansion of adipose tissue is critical for the maintenance of metabolic health, providing an optimized reservoir for energy storage in the form of triacylglycerol-rich lipoproteins. Indeed, dysfunctional adipocytes that are unable to efficiently store lipid can result in lipodystrophy and contribute to non-alcoholic fatty liver disease (NAFLD) and metabolic syndrome. SWELL1 (LRRC8a) is a component of a volume-sensitive membrane protein complex that is highly expressed in adipocytes, induced in the setting of obesity and is required or normal adipocyte expansion during high-fat feeding. Here, we show that adipocyte SWELL1 is required for adipocyte expansion that occurs with high-fat, high-sucrose diet (HFHS, Western diet) and with aging. Adipose-targeted SWELL1 KO mice (Adipo KO) show a mildly reduced body weight over time while raised on a high-fat/high sucrose diet, and no significant body weight difference in aged mice. Similarly, total fat mass and percent fat assessed by NMR is also mildly reduced in obese mice but not in aged mice. However, iWAT and eWAT depot weights are significantly lower in Adipo KO mice compared to WT in both HFHS fed and aged mice. Liver mass is significantly increased in aged and in HFHS-fed Adipo KO mice, and this is associated with significant hepatic steatosis, and NAFLD. These data highlight the importance of adipocyte SWELL1 for healthy adipocyte expansion to protect against NAFLD in the setting of over nutrition and with aging.

Maintenance of adipose tissue is a fundamental process in energy homeostasis and is critical especially in the milieu of changing energy requirements in both healthy and diseased state environment. In previous work, we had discovered a novel transmembrane protein called SWELL1 (also known as LRRC8A) required for volume regulated anion current (VRAC) activity in adipocytes, acts as a sensor for adipocyte size and is required for insulin sensitivity. Adipose specific SWELL1 knockout (SWELL1 KO) mice are insulin insensitive and are subject to decreased adiposity under high-fat diet fed obese conditions when compared to their wildtype (WT) counterparts, demonstrating that SWELL1 is required for expansion of fat. We then questioned how the excess fat is distributed in the absence of SWELL1 by subjecting the WT and SWELL1 KO mice to high-fat/high-sucrose diet for 27 weeks. We then isolated the tissues and found that the KO mice possessed significantly decreased fat content (41% and 9% decrease in eWAT and iWAT respectively) confirming our previous report. Interestingly, we found that the mass of liver in SWELL1 KO mice increased by 32% compared to WT and upon histological examination through Haematoxylin and eosin staining the SWELL1 KO livers were indicative of more steatosis compared to the WT. These data demonstrate that adipose SWELL1 is required for fat maintenance and expansion in the obese conditions without which the fat deposition mechanism becomes dis-regulated and develops into non-alcoholic fatty liver disease.

Obesity results largely from massive volumetric expansion of constituent adipocytes. It is this increase in adipocyte size that is tightly associated with metabolic disease, implicating the action of an undiscovered volume-sensing signaling molecule in adipocytes. Adipocyte patch-clamp was combined with shRNA- and CRISPR/cas9-mediated gene silencing to show that SWELL1 (LRRC8a), a member of the Leucine Rich Repeat Containing protein family, encodes a volume-sensitive current in adipocytes. SWELL1 is induced and activated in hypertrophic adipocytes in the setting of obesity and is required for adipocyte hypertrophy and glucose uptake. Moreover, SWELL1 modulates adipocyte insulin signaling via C-terminal leucine-rich repeat domain interactions with GRB2/Cav1 and PI3K-AKT pathway. In vivo, SWELL1 knock-down reduces adipocyte size, fat mass and exacerbates glucose intolerance in obese mice. These studies identify SWELL1 as a cell-autonomous sensor of adipocyte size that regulates adipocyte growth, insulin sensitivity and glucose tolerance in the setting of obesity.

Further, insulin secretion from the pancreatic 3-cell is initiated by calcium influx through voltage-gated Ca²⁺ channels (VGCC) to trigger insulin vesicle fusion with the 3-cell plasma membrane. The firing of VGCC depends on the 3-cell membrane potential, which is in turn mediated by the balance of depolarizing (excitatory) and hyperpolarizing (inhibitory) ionic currents. While much attention has focused on inhibitory hyperpolarizing potassium currents, there is little knowledge about the requisite excitatory currents required to depolarize the 3-cell, including the molecular identity of these excitatory currents. One candidate for a depolarizing current is a chloride conductance known as the volume-regulatory anion current (VRAC) or I_{Cl,SWELL}. Here it is shown, using shRNA and CRISPR/cas9 gene silencing combined with 3-cell patch-clamp, that SWELL1 (LRRC8a) mediates I_{Cl,SWELL} in β-cells. SWELL1-mediated I_{Cl,SWELL} activates in response to hypotonic and glucose-stimulated β-cell swelling. SWELL1-depletion entirely disrupts both glucose-stimulated and hypotonic swell-mediated activation of VGCC-dependent intracellular calcium signaling in β-cells. Finally, SWELL1 KO MIN6 cells and 0-cell targeted SWELL1 KO murine islets exhibit significantly impaired glucose-stimulated insulin secretion, with preserved insulin content. These results reveal a physiological role for SWELL1 as a glucose sensor—linking glucose-mediated j-cell swelling to SWELL1-dependent activation of VGCC-triggered calcium signaling and insulin secretion. These findings highlight the importance of SWELL1 in swell-mediated f-cell activation, a form of “swell-secretion” coupling important for glucose-stimulated insulin secretion.

### Adipocytes

The adipocyte has been optimized over several hundred million years to maximize energy storage by forming a large lipid droplet, separated from the plasma membrane by only a thin rim (˜300 nm) of cytoplasm, with nucleus and other organelles pushed aside. The adipocyte is also unique in its tremendous capacity for volumetric expansion, increasing by more than 30-fold in the setting of obesity to accommodate the expanding lipid droplet during times of plenty (Farnier et al., Int J Obes Relat Metab Disord, 27, 1178-1186 (2003)). These findings lead to some interesting questions: Could the growing lipid droplet mechanically interact with the plasma membrane to increase membrane tension? Could there be molecular “stretch” sensors active within the adipocyte plasma membrane that may signal to lipid growth pathways? There are several recent studies in the bioengineering field that link adipocyte lipid droplet expansion with increases in adipocyte stiffness and reduced membrane compliance (Shoham et al., Biophys J, 106, 1421-1431 (2014)), in addition to a relationship between membrane tension and activation of adipogenic MAP Kinase signaling pathways (Shoham et al., Am J Physiol Cell Physiol, 302, C429-441 (2012); Pellegrinelli et al., The Journal of pathology, 233, 183-195 (2014)). Moreover, adipocyte size correlates in obesity (as opposed to number) and the severity of linked diseases such as diabetes and insulin resistance (Salans et al., J Clin Invest, 47, 153-165 (1968); Weyer et al., Diabetologia, 43, 1498-1506 (2000); Khan et al., Molecular and cellular biology, 29, 1575-1591 (2009)). Other studies propose that caveolae allow expanding adipocytes to auto-regulate lipid content based on mechanical lipid droplet-plasma membrane interactions and tune insulin signaling in response to adipocyte swelling (Briand et al., Diabetes, 63, 4032-4044 (2014); Eduardsen et al., Cell Physiol Biochem, 28, 1231-1246 (2011)).

Ion channels are membrane proteins that can signal in response to membrane-stretch. There are a number of candidate stretch/mechano-sensitive ion channels in mammalian cells including TRPM7, TRPV2, TRPV4, TRPC6 and Piezo-1/Piezo-2. Many of these ion channels are expressed in adipocytes, and have signaling roles important for adipogenesis, fatty acid sensing, oxidative metabolism, inflammation and energy homeostasis (Che et al., Pflugers Arch, 466, 947-959 (2014); Sukumar et al., Circ Res, 111, 191-200 (2012); Ye et al., Cell, 151, 96-110 (2012)).

As described herein, the swell-activated ion channel signaling in adipocytes was explored by applying the patch-clamp technique to freshly isolated, mature murine and human adipocytes that were mechanically swelled by applying positive pressure intracellularly or by osmotic swelling with hypotonic solution. Using this approach, a prominent swell-activated chloride current (SAC) was discovered in adipocytes encoded by the gene LRRC8a, a member of the Leucine Rich Repeat (LRR) Containing proteins (Voss et al., Science, 344, 634-638 (2014); Qiu et al., Cell, 157, 447-458 (2014)); renamed SWELL1 by Qiu and colleagues. As described herein, it was hypothesized that SWELL1 may sense adipocyte volume during physiological or pathophysiological adipocyte expansion and engage insulin-PI3K-AKT signaling, thereby coupling adipocyte size with growth and insulin sensitivity. Herein, the volume-sensitive SWELL1 molecule is linked to adipocyte insulin signaling, growth and systemic glucose homeostasis. Accordingly, a model is proposed in which SWELL1 tracks adipocyte expansion, and accordingly tunes insulin-mediated activation of growth and glucose import pathways. This discovery allows for the development of improved methods for treating nonalcoholic fatty liver disease (NAFLD).

The swell-activated molecule, SWELL1 is highly expressed in adipocytes, is enriched and activated in the context of adipocyte hypertrophy in obesity, and is required for maintaining adipocyte size, insulin sensitivity and glucose homeostasis via a LRRD-mediated GRB2 interaction with the insulin-PI3K-AKT2 signaling pathway. These findings link the volume-sensitive molecule, SWELL1, with adipocyte insulin-PI3K signaling and provide a molecular mechanism for the effects of adipocyte membrane tension on lipogenesis and intracellular signaling. Based on the data presented herein, a working model is presented in which SWELL1 is activated by increases in adipocyte volume during adipocyte hypertrophy, and this potentiates insulin-PI3K-AKT2 signaling via C-terminal LRRD interactions with GRB2-Cav1-IRS1-IR to support insulin-mediated glucose import and lipogenesis. In this model, SWELL1 senses adipocyte volumetric expansion and acts as a feed-forward amplifier to further promote adipocyte expansion, energy storage, and enhance insulin sensitivity during times of caloric excess (feeding).

While it is tempting to connect SWELL1-mediated channel activity with its signaling role either via SWELL1 conformational changes or ion permeation, and while not intending to be limited by this hypothesis, an alternative possibility is that SWELL1 Leucine-Rich Repeat Domains (LRRD) provide docking surfaces for protein-protein interactions and passively promote the association of components of the insulin signaling cascade (GRB2), or other signaling pathways. In this case there may be no direct relationship between SWELL1-mediated channel activation and insulin-PI3K-AKT signaling. Further, SWELL1 forms heteromultimers with LRRC8b-e, which modifies channel gating, and may also influence the diversity of molecular interactions with different protein partners based on the relative abundance of LRRC8b-e. Therefore it is possible that, depending in the expression profile of LRRC8 proteins in different tissues, SWELL1 modulation of intracellular signaling may vary in a cell-type dependent fashion.

An intriguing observation is that SWELL1 deficiency specifically prevents insulin-PI3K-AKT2 signaling and glucose uptake despite a constitutive increase in AKT1 signaling. Indeed, both cellular and in vivo phenotypes are entirely consistent with previous AKT2-selective loss of function studies, including insulin resistance, reduced adiposity and glucose intolerance. Conversely, AKT1 is dispensable for maintenance of glucose homeostasis but is instead required for organismal growth and development. Moreover, recent work highlights AKT2 over AKT1 as required for adipogenesis in the setting of obesity. Thus, targeting the SWELL1-PI3K-AKT2 axis may represent a novel approach to specifically modulate AKT2 effects on adiposity and insulin sensitivity without altering adipose tissue development. Further molecular studies to determine the mechanism for biased SWELL1-PI3K-AKT2 over AKT1 signaling are also warranted.

The LRRD-mediated SWELL1-GRB2-Cav1 molecular interaction connecting SWELL1 to insulin-PI3K-AKT2 signaling is both consistent with the SWELL1 (LRRC8a)-GRB2-GAB2-LCK complex reported in lymphocytes and also provides a molecular mechanism for the observed defect in insulin-PI3K-AKT2 signaling upon adipocyte SWELL1 ablation. Likewise, the SWELL1-Cav1 interaction is also compelling as this positions SWELL1 within caveolae, which are abundant in adipocytes, are thought to form insulin-signaling microdomains and are required for normal insulin and PI3K-AKT signaling. Indeed, Cav1 KO mice on a HFD are phenotypically similar to AAV/Rec2-mediated SWELL1 KD mice with respect to adiposity and insulin-sensitivity.

Insulin-stimulation reduces both SWELL1-GRB2 and Cav1-GRB2, but not SWELL1-Cav1 interactions in WT 3T3-F442A adipocytes. This suggests that insulin-stimulation induces GRB2 dissociation from the insulin-signaling complex. Curiously, this insulin-mediated GRB2 dissociation from Cav1 is abrogated upon SWELL1 ablation implying that SWELL1 may tune insulin signaling by titrating GRB2-interactions with components of the insulin signaling complex. This intriguing hypothesis warrants further investigation.

### B-Cells

Normal SWELL1 function is required for normal human immune system development. In one example, expression of a truncated SWELL1 protein caused by a translocation in one allele of SWELL1 inhibits normal B-cell development, causing agammaglobulinemia 5 (AGM5). (Sawada et al., J Clin Invest. 2003 December; 112(11):1707-13; Kubota et al., FEBS Lett. 2004 Apr. 23; 564(1-2):147-52) Because different types of immune system cells (e.g. B-lymphocytes and T-lymphocytes) use similar intracellular signaling pathways, it is likely that the development and/or function of other immune system cells (e.g. T-lymphocytes, macrophages, and/or NK cells) would also be affected by inadequate SWELL1 expression or function. A therapy that could increase expression and/or function of SWELL1 could be used to treat immune deficiencies due to SWELL1-based abnormal immune cell development and/or function.

### Spermatazoa

Currently, the molecular causes of male infertility are only partially understood. In mice lacking SWELL1 late spermatids fail to reduce their cytoplasm during development into spermatozoa and have disorganized mitochondrial sheaths with angulated flagella, resulting in reduced sperm motility. This demonstrates that SWELL1 is also required for normal spermatid development and male fertility. (Luck et al., J Biol Chem. 2018 Jul. 27; 293(30):11796-11808) A therapy that could increase expression or function of SWELL1 could be used to treat male infertility due to SWELL1-based abnormal spermatozoa development and/or function.

### SWELL1 Modulators

Embodiments of the present invention are directed to the use of SWELL1 modulators to treat diseases such as nonalcoholic fatty liver disease (NAFLD) and to regulate vascular tone, systemic arterial and/or pulmonary arterial blood pressure and/or blood flow.

### DCPIB

DCPIB (4-[(2-Butyl-6,7-dichloro-2-cyclopentyl-2,3-dihydro-1-oxo-1H-inden-5-yl)oxy]butanoic acid), a selective SWELL1 inhibitor, is a potent and selective inhibitor of the volume-sensitive anion channel (VSAC) in rat pancreatic β-cells and I_{Cl,swell} in various cardiovascular tissues. DCPIB is an example of a SWELL1 modulator useful in the practice of certain embodiments of the invention.

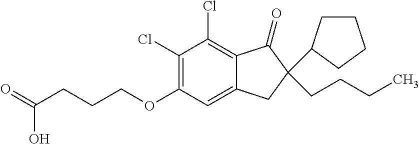 [SMILES: CCCCC1(C2CCCC2)Cc2cc(OCCCC(=O)O)c(Cl)c(Cl)c2C1=O]

Other SWELL1 modulators include clomiphene, nafoxidine and tamoxifen and compounds as described below, or salts thereof.

Accordingly, in certain embodiments the SWELL1 modulator is a compound of formula I

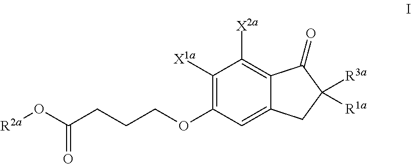

wherein:

X^{1a} and X^{2a} are independently halo;

R^{1a} is C₁₋₆ alkyl, 3-6 membered cycloalkyl, or phenyl, wherein the C₁₋₆ alkyl is optionally substituted with 3-6 membered cycloalkyl;

R^{2a} is hydrogen or C₁₋₆ alkyl, wherein the C₁₋₆ alkyl is optionally substituted with carboxy; and

R^{3a} is C₁₋₆ alkyl;

or a pharmaceutically acceptable salts thereof.

In certain embodiments, the compound is a compound of the following formula, or a salt thereof (see: U.S. Pat. No. 4,465,850)

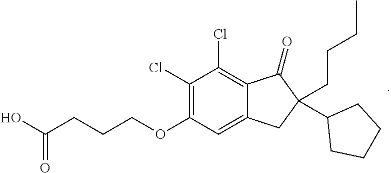 [SMILES: CCCCC1(C2CCCC2)Cc2cc(OCCCC(=O)O)c(Cl)c(Cl)c2C1=O]

In certain embodiments the SWELL1 modulator is a compound of formula II

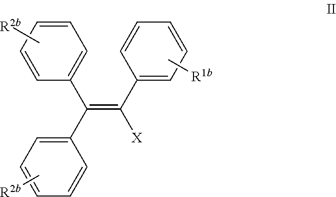

wherein:

R^{1b} is hydrogen, halo or methoxy;

only one of R^{2b} is —O(CH₂)ₙ—NR^{3b}R^{4b};

the other R^{2b} is hydrogen, halo or methoxy;

each of R^{3b} and R^{4b} is independently H or C₁₋₆ alkyl, or R^{3b} and R^{4b} together with the nitrogen to which they are attached form aziridino, azetidino, morpholino, piperazino, pyrrolidino or piperidino;

n is an integer from 2 to 4; and

X is halo;

or a pharmaceutically acceptable salt thereof.

In certain embodiments, the compound is a compound of the following formula, or a salt thereof. (see U.S. Pat. No. 2,914,563)

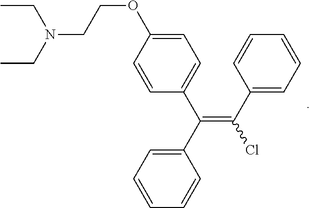 [SMILES: CCN(CC)CCOc1ccc(C(=C(Cl)c2ccccc2)c2ccccc2)cc1]

In certain embodiments the SWELL1 modulator is a compound of formula III

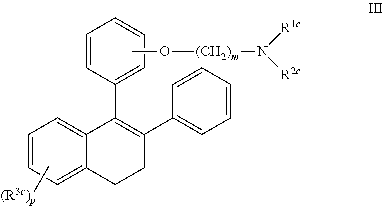

wherein:

each of R^{1c} and R^{2c} is independently H or C₁₋₈ alkyl, or R^{1c} and R^{2c} together with the nitrogen to which they are attached form aziridino, azetidino, morpholino, piperazino, pyrrolidino or piperidino; wherein the aziridino, azetidino, morpholino, piperazino, pyrrolidino and piperidino are optionally substituted with one or more C₁₋₆ alkyl;

m is an integer from 2 to 6,

R^{3c} is C₁₋₈ alkoxy; and

p is an integer from 1 to 4;

or a pharmaceutically acceptable salt thereof.

In certain embodiments, the compound is a compound of the following formula, or a salt thereof.
(see U.S. Pat. No. 3,274,213)

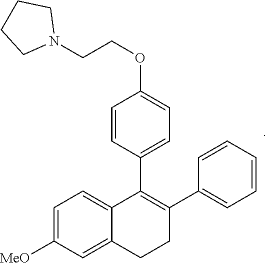 [SMILES: COc1ccc2c(c1)CCC(c1ccccc1)=C2c1ccc(OCCN2CCCC2)cc1]

In certain embodiments the SWELL1 modulator is a compound of formula IV

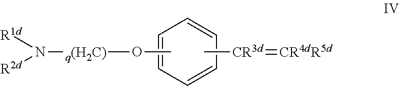

wherein:

each of R^{1d} and R^{2d} is independently H or C₁₋₆ alkyl, or R^{1d} and R^{2d} together with the nitrogen to which they are attached form aziridino, azetidino, morpholino, piperazino, pyrrolidino or piperidino;

each of R^{3d} and R^{4d} is independently aryl which is optional substituted with one or more groups selected from C₁₋₆ alkyl, C₁₋₆ alkoxy, C₁₋₆ dialkylamino, or halo;

R^{5d} is C₁₋₆ alkyl or C₁₋₆ alkenyl, wherein the C₁₋₆ alkyl is optionally substituted with aryl; and

q is an integer from 2 to 6;

or a pharmaceutically acceptable salt thereof.

In certain embodiments, the compound is a compound of the following formula, or a salt thereof. (see U.S. Pat. No. 4,536,516)

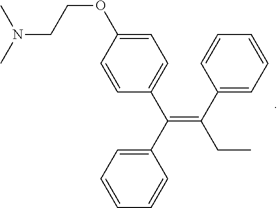 [SMILES: CC/C(=C(\c1ccccc1)c1ccc(OCCN(C)C)cc1)c1ccccc1]

“Systemic delivery,” as used herein, refers to delivery of agents that lead to a broad biodistribution of an active agent within an organism. Some techniques of administration can lead to the systemic delivery of certain agents, but not others. Systemic delivery means that a useful, preferably therapeutic, amount of an agent is exposed to most parts of the body. To obtain broad biodistribution generally requires a blood lifetime such that the agent is not rapidly 10 degraded or cleared (such as by first pass organs (liver, lung, etc.) or by rapid, nonspecific cell binding) before reaching a desired site distal to the site of administration. Systemic delivery of active agents (e.g., SWELL1 modulators) can be by any means known in the art including, for example, intravenous, subcutaneous, and intraperitoneal. In a preferred embodiment, systemic delivery is by intravenous delivery.

“Local delivery,” as used herein, refers to delivery of an active agent such as a siRNA directly to a target site within an organism. For example, an agent can be locally delivered by direct injection into a disease site, other target site, or a target organ such as the liver, heart, pancreas, kidney, and the like.

The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

An “effective amount” of an agent, e.g., a pharmaceutical formulation, refers to an amount effective, at dosages and for periods of time necessary, to achieve the desired therapeutic or prophylactic result. In some embodiments, the effective amount refers to an amount of a SWELL1 modulator that (i) treats the particular disease, condition or disorder, (ii) attenuates, ameliorates or eliminates one or more symptoms of the particular disease, condition, or disorder, or (iii) prevents or delays the onset of one or more symptoms of the particular disease, condition or disorder described herein.

“Treatment” (and variations such as “treat” or “treating”) refers to clinical intervention in an attempt to alter the natural course of the individual or cell being treated, and can be performed either for prophylaxis or during the course of clinical pathology. Desirable effects of treatment include one or more of preventing occurrence or recurrence of disease, alleviation of symptoms, diminishment of any direct or indirect pathological consequences of the disease, stabilized (i.e., not worsening) state of disease, decreasing the rate of disease progression, amelioration or palliation of the disease state, prolonging survival as compared to expected survival if not receiving treatment and remission or improved prognosis. In certain embodiments, a SWELL1 modulator is used to delay development of a disease or disorder or to slow the progression of a disease or disorder. Those individuals in need of treatment include those already with the condition or disorder as well as those prone to have the condition or disorder, (for example, through a genetic mutation or aberrant expression of a gene or protein) or those in which the condition or disorder is to be prevented.

As used herein, “delaying progression of a disease” means to defer, hinder, slow, retard, stabilize, and/or postpone development of the disease (such as nonalcoholic fatty liver disease (NAFLD)). This delay can be of varying lengths of time, depending on the history of the disease and/or individual being treated. As is evident to one skilled in the art, a sufficient or significant delay can, in effect, encompass prevention, in that the individual does not develop the disease.

Further provided herein are pharmaceutical compositions that comprise a SWELL1 modulator for use in the methods described herein, e.g., to treat nonalcoholic fatty liver disease (NAFLD). In one embodiment, the composition further comprises a pharmaceutically acceptable carrier, adjuvant, or vehicle. In another embodiment, the composition further comprises an amount of the compound effective to measurably inhibit SWELL1, modulate SWELL1 activity or increase SWELL1 expression level, or associated protein partners. In certain embodiments, the composition is formulated for administration to a patient in need thereof.

Compositions comprising a SWELL1 modulator or salt thereof may be administered orally, parenterally, by inhalation spray, topically, transdermally, rectally, nasally, buccally, sublingually, vaginally, intraperitoneal, intrapulmonary, intradermal, epidural or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques.

In one embodiment, the composition comprising a SWELL1 modulator or salt thereof is formulated as a solid dosage form for oral administration. Solid dosage forms for oral administration include capsules, tablets, pills, powders, and granules. In certain embodiments, the solid oral dosage form comprising a SWELL1 modulator or a salt thereof further comprises one or more of (i) an inert, pharmaceutically acceptable excipient or carrier, such as sodium citrate or dicalcium phosphate, and (ii) filler or extender such as starches, lactose, sucrose, glucose, mannitol, or silicic acid, (iii) binders such as carboxymethylcellulose, alginates, gelatin, polyvinylpyrrolidinone, sucrose or acacia, (iv) humectants such as glycerol, (v) disintegrating agent such as agar, calcium carbonate, potato or tapioca starch, alginic acid, certain silicates or sodium carbonate, (vi) solution retarding agents such as paraffin, (vii) absorption accelerators such as quaternary ammonium salts, (viii) a wetting agent such as cetyl alcohol or glycerol monostearate, (ix) absorbent such as kaolin or bentonite clay, and (x) lubricant such as talc, calcium stearate, magnesium stearate, polyethylene glycols or sodium lauryl sulfate. In certain embodiments, the solid oral dosage form is formulated as capsules, tablets or pills. In certain embodiments, the solid oral dosage form further comprises buffering agents. In certain embodiments, such compositions for solid oral dosage forms may be formulated as fillers in soft and hard-filled gelatin capsules comprising one or more excipients such as lactose or milk sugar, polyethylene glycols and the like.

In certain embodiments, tablets, dragees, capsules, pills and granules of the compositions comprising a SWELL1 modulator or salt thereof optionally comprise coatings or shells such as enteric coatings. They may optionally comprise opacifying agents and can also be of a composition that they release the active ingredient(s) only, or preferentially, in a certain part of the intestinal tract, optionally, in a delayed manner. Examples of embedding compositions include polymeric substances and waxes, which may also be employed as fillers in soft and hard-filled gelatin capsules using such excipients as lactose or milk sugar as well as high molecular weight polethylene glycols and the like.

In another embodiment, a composition comprises a micro-encapsulated SWELL1 modulator or salt thereof, and optionally, further comprises one or more excipients.

In another embodiment, compositions comprise liquid dosage formulations comprising a SWELL1 modulator or salt thereof for oral administration, and optionally further comprise one or more of pharmaceutically acceptable emulsions, microemulsions, solutions, suspensions, syrups and elixirs. In certain embodiments, the liquid dosage form optionally, further comprise one or more of an inert diluent such as water or other solvent, a solubilizing agent, and an emulsifier such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols or fatty acid esters of sorbitan, and mixtures thereof. In certain embodiments, liquid oral compositions optionally further comprise one or more adjuvant, such as a wetting agent, a suspending agent, a sweetening agent, a flavoring agent and a perfuming agent.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a SWELL1 modulator, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

In certain embodiments, the composition for rectal or vaginal administration are formulated as suppositories which can be prepared by mixing a SWELL1 modulator or a salt thereof with suitable non-irritating excipients or carriers such as cocoa butter, polyethylene glycol or a suppository wax, for example those which are solid at ambient temperature but liquid at body temperature and therefore melt in the rectum or vaginal cavity and release the SWELL1 modulator.

Example dosage forms for topical or transdermal administration of a SWELL1 modulator include ointments, pastes, creams, lotions, gels, powders, solutions, sprays, inhalants or patches. The SWELL1 modulator or a salt thereof is admixed under sterile conditions with a pharmaceutically acceptable carrier, and optionally preservatives or buffers. Additional formulation examples include an ophthalmic formulation, ear drops, eye drops, transdermal patches. Transdermal dosage forms can be made by dissolving or dispensing the SWELL1 modulator or a salt thereof in medium, for example ethanol or dimethylsulfoxide. Absorption enhancers can also be used to increase the flux of the compound across the skin. The rate can be controlled by either providing a rate controlling membrane or by dispersing the compound in a polymer matrix or gel.

Nasal aerosol or inhalation formulations of a SWELL1 modulator or a salt thereof may be prepared as solutions in saline, employing benzyl alcohol or other suitable preservatives, absorption promotors to enhance bioavailability, fluorocarbons, and/or other conventional solubilizing or dispersing agents.

In certain embodiments, pharmaceutical compositions may be administered with or without food. In certain embodiments, pharmaceutically acceptable compositions are administered without food. In certain embodiments, pharmaceutically acceptable compositions of this invention are administered with food.

Specific dosage and treatment regimen for any particular patient will depend upon a variety of factors, including age, body weight, general health, sex, diet, time of administration, rate of excretion, drug combination, the judgment of the treating physician, and the severity of the particular disease being treated. The amount of a provided SWELL1 modulator or salt thereof in the composition will also depend upon the particular compound in the composition.

In one embodiment, the effective amount of the compound administered parenterally per dose will be in the range of about 0.01-100 mg/kg, alternatively about 0.1 to 20 mg/kg of patient body weight per day, with the typical initial range of compound used being 0.3 to 15 mg/kg/day. In another embodiment, oral unit dosage forms, such as tablets and capsules, contain from about 5 to about 100 mg of the compound of the invention.

An example tablet oral dosage form comprises about 2 mg, 5 mg, 25 mg, 50 mg, 100 mg, 250 mg or 500 mg of a SWELL1 modulator or salt thereof, and further comprises about 5-30 mg anhydrous lactose, about 5-40 mg sodium croscarmellose, about 5-30 mg polyvinylpyrrolidone (PVP) K30 and about 1-10 mg magnesium stearate. The process of formulating the tablet comprises mixing the powdered ingredients together and further mixing with a solution of the PVP. The resulting composition can be dried, granulated, mixed with the magnesium stearate and compressed to tablet form using conventional equipment. An example of an aerosol formulation can be prepared by dissolving about 2-500 mg of a compound of formula I or salt thereof, in a suitable buffer solution, e.g. a phosphate buffer, and adding a tonicifier, e.g. a salt such sodium chloride, if desired. The solution may be filtered, e.g. using a 0.2 micron filter, to remove impurities and contaminants.

The SWELL1 inhibitors or modulators or salts thereof may be employed alone or in combination with other agents for treatment as described above. For example, the second agent of the pharmaceutical combination formulation or dosing regimen may have complementary activities to the SWELL1 modulator such that they do not adversely affect each other. The compounds may be administered together in a unitary pharmaceutical composition or separately.

The term “co-administering” refers to either simultaneous administration, or any manner of separate sequential administration, of a SWELL1 modulator or a salt thereof, and a further active pharmaceutical ingredient or ingredients. If the administration is not simultaneous, the compounds are administered in a close time proximity to each other. Furthermore, it does not matter if the compounds are administered in the same dosage form, e.g. one compound may be administered topically and another compound may be administered orally. Typically, any agent that has activity against a disease or condition being treated may be co-administered.

The term “patient” or “individual” as used herein, refers to an animal, such as a mammal, such as a human. In one embodiment, patient or individual refers to a human.

The term “mammal” refers to any mammalian species such as a human, mouse, rat, dog, cat, hamster, guinea pig, rabbit, livestock, and the like.

Recitation of ranges of values herein are merely intended to serve as a shorthand method of referring individually to each separate value falling within the range, unless otherwise indicated herein, and each separate value is incorporated into the specification as if it were individually recited herein.

As is understood by one skilled in the art, reference to “about” a value or parameter herein includes (and describes) embodiments that are directed to that value or parameter per se. For example, description referring to “about X” includes description of “X”.

The use of the terms “a” and “an” and “the” and similar terms in the context of describing embodiments of invention are to be construed to cover both the singular and the plural, unless otherwise indicated herein or clearly contradicted by context. The terms “comprising,” “having,” “including,” and “containing” are to be construed as open-ended terms (i.e., meaning “including, but not limited to”) unless otherwise noted. It is understood that aspect and embodiments of the invention described herein include “consisting” and/or “consisting essentially of” aspects and embodiments.

Certain embodiments of the invention will now be illustrated by the following non-limiting Examples.

### Example 1

Type 2 diabetes (T2D) is characterized by both a loss of insulin sensitivity of target tissues and ultimately, impaired insulin secretion from the pancreatic β-cell (Del Guerra et al., Diabetes, 54, 727-735, 2005; Ashcroft et al., Cell, 148, 1160-1171, 2012; Rorsman et al., Annu Rev Physiol, 75, 155-179, 2013). Recent findings that SWELL1 (LRRC8a) ablation impairs adipose insulin-pAKT2 signaling (Zhang et al., Nat Cell Biol, 19, 504-517, 2017), f-cell insulin secretion (Kang et al., Nat Commun, 9, 367, 2018; Stuhlmann et al., Nat Commun, 9, 1974, 2018) and systemic glycemia (Xie et al., Channels (Austin), 11(6): 673-677, 2017; Zhang et al., Nat Cell Biol, 19, 504-517, 2017; Kang et al., Nat Commun, 9, 367, 2018; Stuhlmann et al., Nat Commun, 9, 1974, 2018) suggests that SWELL1 dysfunction may contribute to T2D pathogenesis (Xie et al., Channels (Austin), 11(6): 673-677, 2017; Osei-Owusu et al., Curr Top Membr, 81, 177-203, 2018). Here, it is shown that I_{Cl,SWELL}/SWELL1 protein is reduced in T2D β-cells and adipocytes, and that SWELL1 protein expression regulates insulin-AKT2-AS160 signaling. DCPIB (Smod1), a selective I_{Cl,SWELL} inhibitor, upregulates SWELL1 protein, thereby enhancing SWELL1-dependent insulin signaling in cultured adipocytes. In vivo, Smod1 (5 mg/kg i.p.), augments SWELL1 expression and normalizes systemic glycemia in two T2D mouse models (HFD-induced and KKA^{y}) by enhancing both systemic insulin sensitivity and insulin secretion from pancreatic islets, without causing hypoglycemia in non-T2D mice. Smod1 treatment augments glucose uptake in white adipose tissue and myocardium, increases hepatic, adipose and skeletal muscle incorporation of glucose into glycogen, suppresses hepatic glucose production, and improves non-alcoholic fatty liver disease (NAFLD) in T2D mice. These findings reveal that small molecule SWELL1 modulators may represent a “first-in-class” therapeutic approach to treat T2D, metabolic syndrome, and associated NAFLD by concomitantly enhancing systemic insulin-sensitivity and insulin secretion.

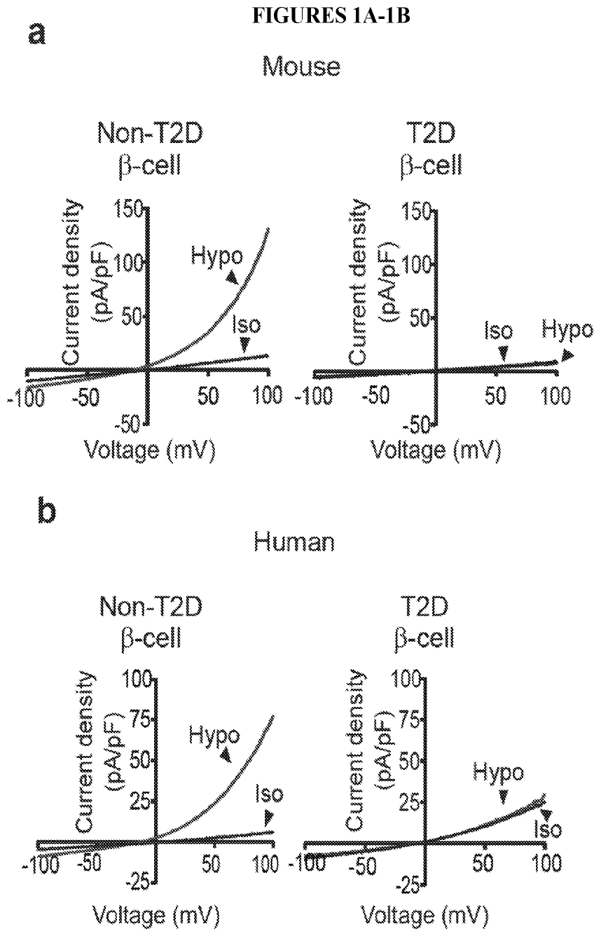

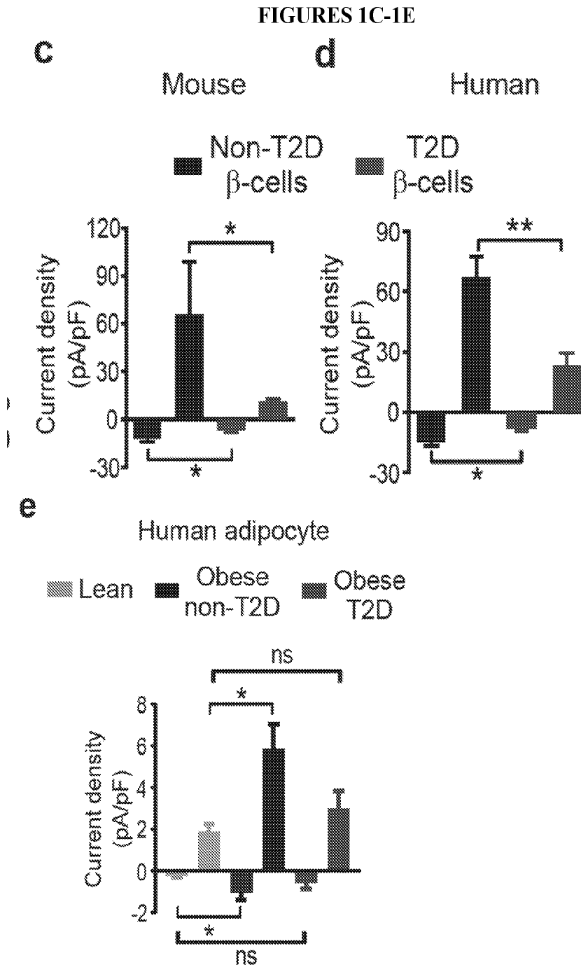

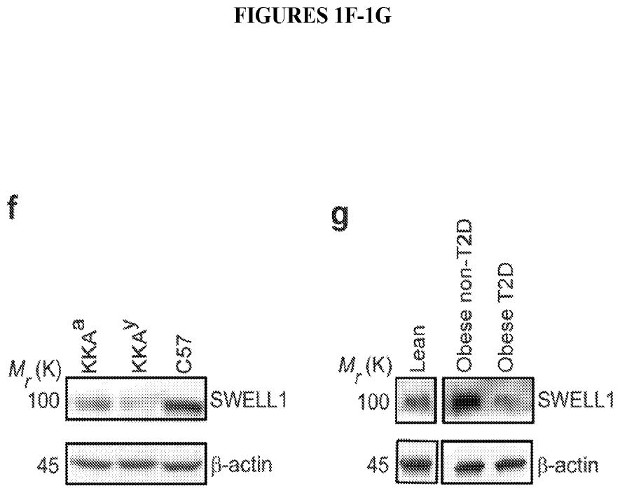

SWELL1/LRRC8a ablation impairs insulin signaling in target tissues (Xie et al., Channels (Austin), 11(6): 673-677, 2017; Zhang et al., Nat Cell Biol, 19, 504-517, 2017) and insulin secretion from the pancreatic β-cell (Kang et al., Nat Commun, 9, 367, 2018; Stuhlmann et al., Nat Commun, 9, 1974, 2018), inducing a pre-diabetic state of glucose intolerance (Xie et al., Channels (Austin), 11(6): 673-677, 2017; Zhang et al., Nat Cell Biol, 19, 504-517, 2017; Kang et al., Nat Commun, 9, 367, 2018). These recent findings suggest that reductions in SWELL1 may contribute to Type 2 diabetes (T2D). To determine if SWELL1-mediated currents are altered in T2D, we measured I_{Cl,SWELL} in pancreatic β-cells freshly isolated from T2D mice raised on HFD for 5-7 months (FIG. 1a) and from T2D patients (FIG. 1b, Table 1&2) compared to non-T2D controls. In both mouse and human T2D β-cells, the maximum I_{Cl,SWELL} current density (measured at +100 mV) upon stimulation with hypotonic swelling is significantly reduced (5.9-fold murine; 2.7-fold human, FIG. 1c&d) compared to non-T2D controls, similar to reductions observed in SWELL1 knock-out (KO) and knock-down (KD) murine and human 0-cells (Kang et al., Nat Commun, 9, 367, 2018), respectively. These reductions in β-cell I_{Cl,SWELL} in the setting of T2D are consistent with previous measurements of VRAC/I_{Cl,SWELL} in the murine KKA^{y} T2D model (Inoue et al., Am J Physiol Cell Physiol, 298, C900-909, 2010), which were >2-fold lower I_{Cl,SWELL} in adipocytes isolated from T2D KKA^{y} mice compared to non-T2D controls (Inoue et al., Am J Physiol Cell Physiol, 298, C900-909, 2010). Likewise, SWELL1-mediated I_{Cl,SWELL} measured in isolated human adipocytes from an obese T2D patient (BMI=52.3, HgbAlc=6.9%; Glucose=148-151 mg/dl) show a trend toward being reduced 1.9-fold compared to obese, non-T2D patients that we reported previously (Zhang et al., Nat Cell Biol, 19, 504-517, 2017), and not different from I_{Cl,SWELL} in adipocytes from lean patients (FIG. 1e, Table 3). As SWELL1/LRRC8a is a critical component of I_{Cl,SWELL}/VRAC (Qiu et al., Cell, 157, 447-458, 2014; Voss et al., Science, 344, 634-638, 2014) in both adipose tissue (Xie et al., Channels (Austin), 11(6): 673-677, 2017; Zhang et al., Nat Cell Biol, 19, 504-517, 2017) and 0-cells (Kang et al., Nat Commun, 9, 367, 2018; Stuhlmann et al., Nat Commun, 9, 1974, 2018), we asked whether these reductions in I_{Cl,SWELL} in the setting of T2D (Inoue et al., Am J Physiol Cell Physiol, 298, C900-909, 2010) are associated with reductions in SWELL1 protein expression. Indeed, SWELL1 protein expression is reduced in adipose tissue of a T2D KKA^{y} mouse as compared to either the parental control KKA^{a} or C57BL/6 mouse (FIG. 1f). Similarly, SWELL1 protein is lower in adipose tissue from an obese T2D patient (BMI=53.7, HgbAlc=8.0%, Glucose=183-273 mg/dl) compared to adipose tissue from a normoglycemic obese patient (BMI=50.2 HgbAlc=5.0%; glucose=84-97 mg/dl, FIG. 1g, Table 4). Taken together, these findings suggest that reduced SWELL1 activity in adipocytes and f-cells (and possibly other tissues) may underlie insulin-resistance and impaired insulin secretion associated with T2D. This notion is consistent with the observation that LRRC8a expression is reduced in β-cells from hyperglycemic compared to normoglycemic individuals (Taneera et al., Cell Metab, 16, 122-134, 2012; Osei-Owusu et al., Curr Top Membr, 81, 177-203, 2018). Moreover, SWELL1 protein expression increases in both adipose tissue and liver in the setting of early euglycemic obesity (Xie et al., Channels (Austin), 11(6): 673-677, 2017) and shRNA-mediated suppression of this SWELL1 induction exacerbates insulin-resistance and glucose intolerance (Xie et al., Channels (Austin), 11(6): 673-677, 2017). Therefore, we speculate that maintenance or induction of SWELL1 expression/signaling in peripheral tissues may support insulin sensitivity and secretion to preserve systemic glycaemia in the setting of T2D.

TABLE 1
Characteristics of non-T2D and T2D mice (HFD) from which murine β-cells were
isolated for β-cell patch-clamp in FIG. 1a&c.

| Mouse | Age (weeks) | Sex | Diet | Body weight (g) | Glucose (mg/dl) |
|---|---|---|---|---|---|
| Non-T2D | 12-13 (n = 4) | M | Regular chow | 28.6 ± 0.51 | 148 ± 6.49 |
| T2D | 23-27 (n = 3) | M | High-fat diet | 52.7 ± 2.99 | 229 ± 21.4 |

TABLE 2
Non-T2D and T2D patients from whom β-cells
were isolated for patch-clamp in FIG. 1 b&d.

| | | | | Random | Estimated | |
|---|---|---|---|---|---|---|
| | Age | | | Glucose | Glucose | HbA1c |
| Patient | (years) | Sex | BMI | (mg/dl) | (mg/dl) | (%) |
| Non- | 44 | F | 26.8 | 151.8 | NA | 6.1 |
| T2D | 57 | M | 28.7 | 144.3 | NA | 5.3 |
| | 24 | F | 32.2 | 234 | NA | NA |
| T2D | 46 | F | 35.9 | 262.4 | NA | 6.8 |
| | 37 | F | 38.1 | 253.8 | NA | 8.2 |
| | 51 | M | 35.59 | NA | 157 | 7.1 |

TABLE 3
Lean, non-T2D and T2D bariatric surgery patients
from whom primary adipocytes were isolated for
patch-clamp studies FIG. 1e. ^{#}Data from lean and obese non-
T2D patients were reported previously in Zhang, Y et al. (2017).

| | | | | Random | Estimated | |
|---|---|---|---|---|---|---|
| | Age | | | Glucose | Glucose | HbA1c |
| Patient | (years) | Sex | BMI | (mg/dl) | (mg/dl) | (%) |
| Lean^{#} | 52 | M | 27.56 | 97 | 111 | 5.5 |
| | 61 | F | 28.36 | 112 | NA | 5.5 |
| Obese | 38 | F | 55.10 | 88 | 117 | 5.7 |
| non-T2D^{#} | 65 | F | 32.02 | 100 | 111 | 5.5 |
| | 51 | F | 48.80 | 97 | 114 | 5.6 |
| Obese-T2D | 41 | F | 52.31 | 148 | 151 | 6.9 |

TABLE 4
Lean, obese non-T2D and obese T2D patients
from whom adipose samples were obtained to measure
SWELL1 protein expression levels in FIG. 1g.

| | | | | Random | Estimated | |
|---|---|---|---|---|---|---|
| | Age | | | Glucose | Glucose | HbA1c |
| Patient | (years) | Sex | BMI | (mg/dl) | (mg/dl) | (%) |
| Lean | 47 | F | 24.85 | 97 | 111 | 5.5 |
| Obese | 48 | F | 50.18 | 84 | 97 | 5 |
| non-T2D | | | | | | |
| Obese- | 57 | F | 53.69 | 273 | 183 | 8 |
| T2D | | | | | | |

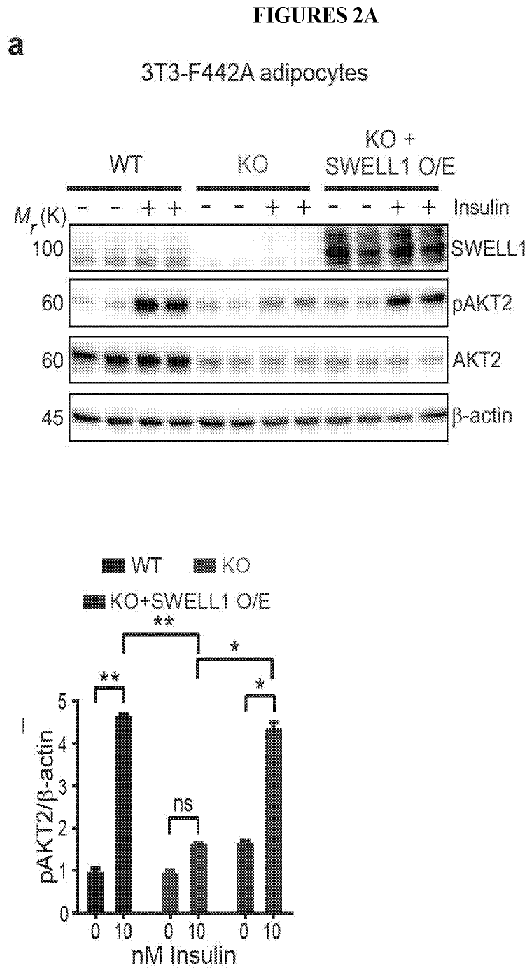

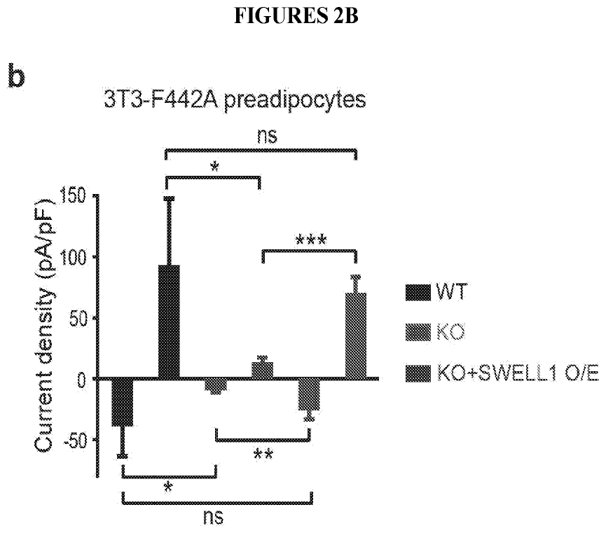

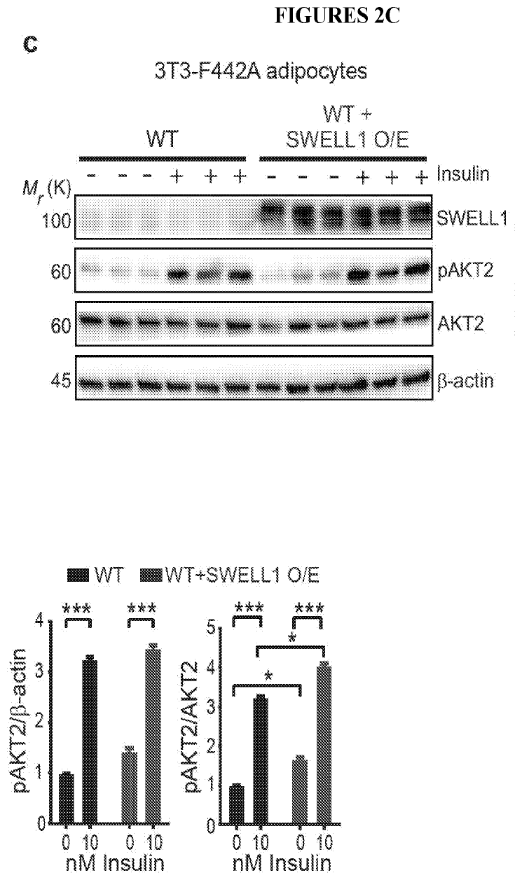

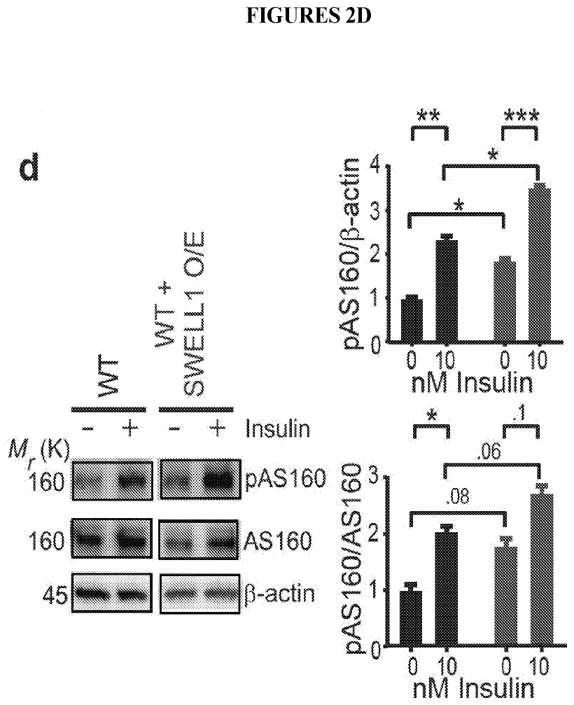

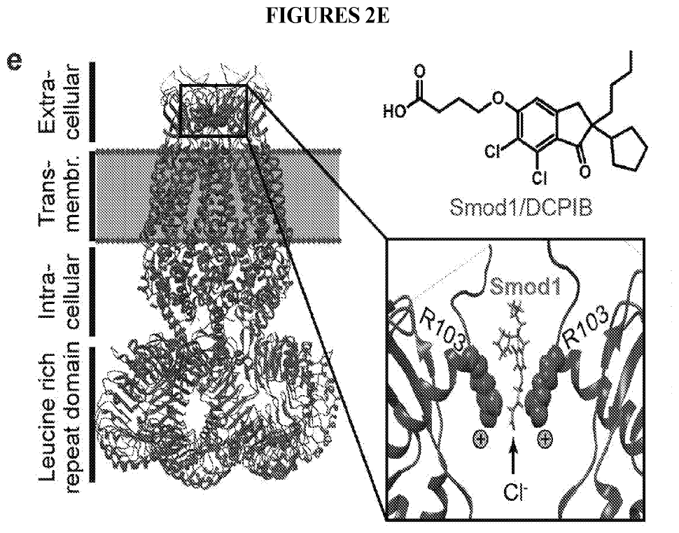

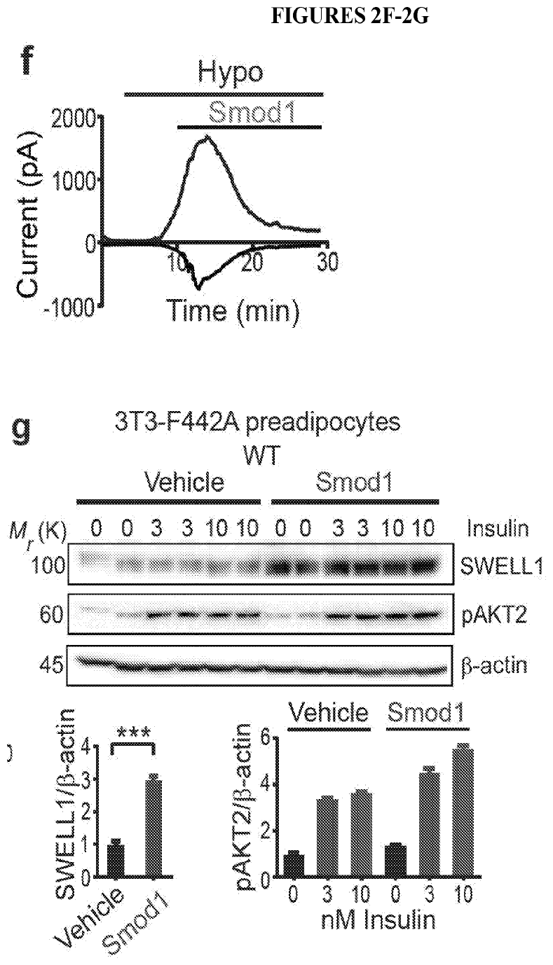

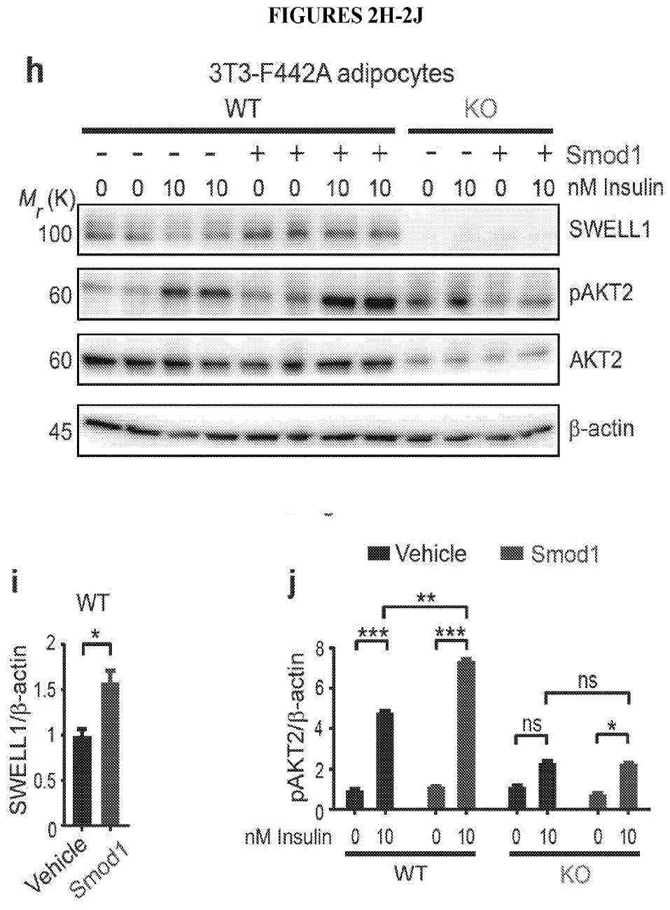

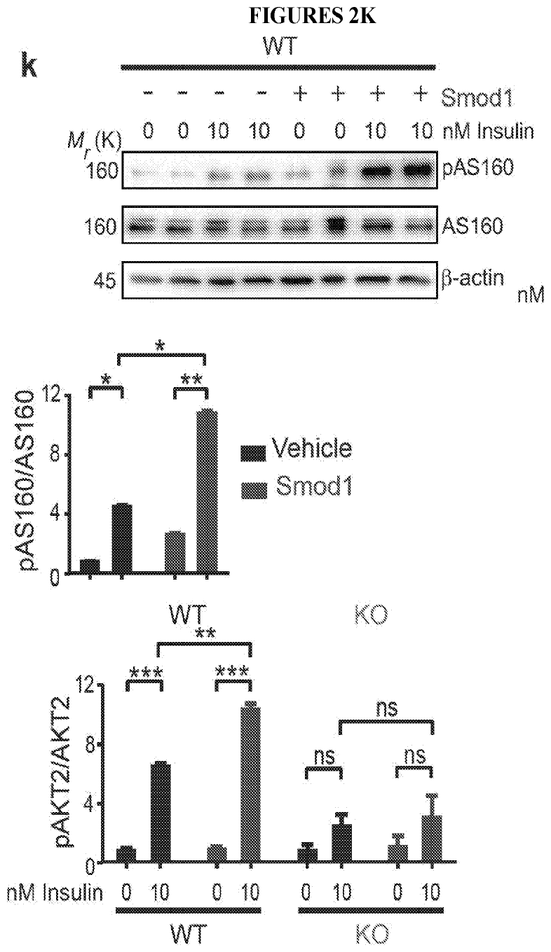

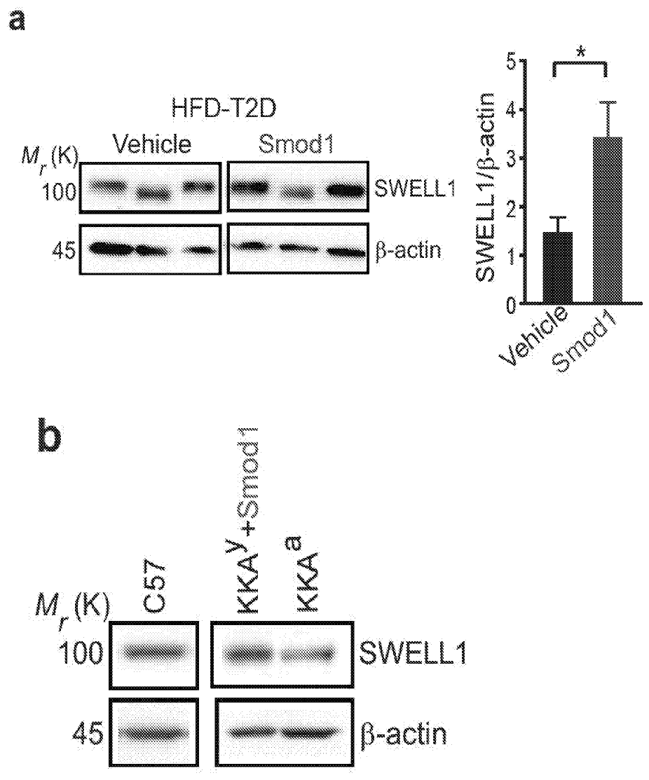

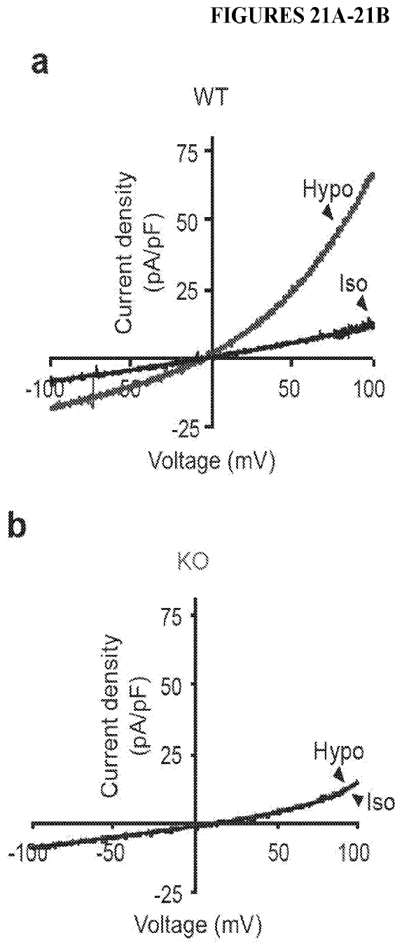

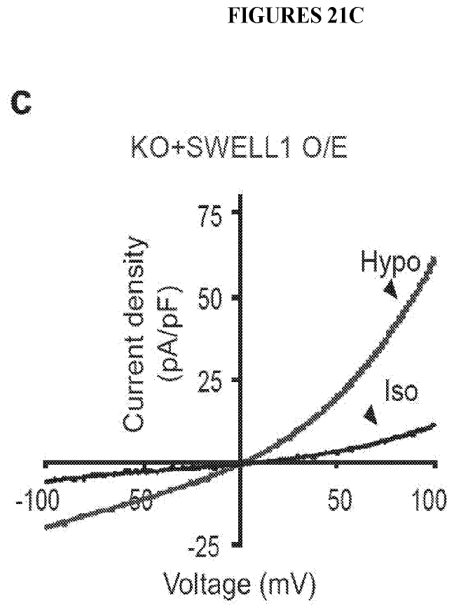

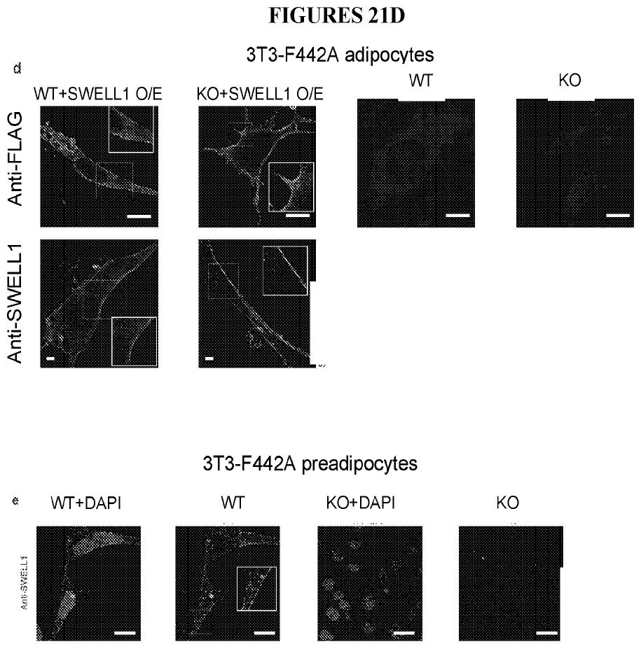

To first test the concept that SWELL1 regulates insulin signaling we overexpressed full-length SWELL1-3× Flag-tagged (SWELL1 O/E) in both WT and SWELL1 KO 3T3-F442A adipocytes and measured insulin-stimulated pAKT2 as a readout of insulin-sensitivity (FIG. 2a). SWELL1 KO 3T3-F442A adipocytes exhibit significantly blunted insulin-mediated pAKT2 signaling compared to WT adipocytes, as described previously (Zhang et al., Nat Cell Biol, 19, 504-517, 2017), and this is fully rescued by re-expression of SWELL1 in SWELL1 KO adipocytes (KO+SWELL1 O/E, FIG. 2a), along with restoring SWELL1-mediated I_{Cl,SWELL} in response to hypotonic stimulation (FIG. 2b and FIG. 21a-c). Notably, the reductions in total AKT2 protein expression observed in SWELL1 KO adipocytes is not rescued by SWELL1 re-expression, indicating that transient changes in SWELL1 protein expression preferentially regulates insulin-pAKT2 signaling, as opposed to AKT2 protein expression. SWELL1 overexpression in WT adipocytes also increases both basal and insulin-stimulated pAKT2 and downstream pAS160 signaling in WT adipocytes (FIG. 2c&d). We confirmed that SWELL1-3× Flag traffics normally to the plasma membrane when expressed in both WT and SWELL1 KO adipocytes as visualized by immunofluorescence (IF) using both anti-Flag and anti-SWELL1 antibodies (FIG. 21d). Indeed, the characteristic punctate pattern of plasma membrane SWELL1-3× Flag expressed in WT and SWELL1 KO adipocytes (FIG. 21d) closely resembles the pattern of endogenous SWELL1, visualized by IF using our knockout validated, custom-made SWELL1 antibody (FIG. 21e). Overall, these data indicate that SWELL1 is both necessary and sufficient for insulin-PI3K-AKT2-AS160 signaling in adipocytes. Importantly, these data suggest that pharmacological SWELL1 induction in peripheral tissues in the setting of T2D may enhance insulin signaling, and improve systemic insulin-sensitivity and glucose homeostasis.

4-[(2-Butyl-6,7-dichloro-2-cyclopentyl-2,3-dihydro-1-oxo-1H-inden-5-yl)oxy]butanoic acid (DCPIB, FIG. 2e) is among a series of structurally diverse (acylaryloxy)acetic acid derivatives that were synthesized and studied for diuretic properties in the late 1970s (Woltersdorf et al., J Med Chem, 20, 1400-1408, 1977; deSolms et al., J Med Chem, 21, 437-443, 1978), and evaluated in the 1980s as potential treatments for brain edema (Cragoe et al., J Med Chem, 25, 567-579, 1982; Cragoe et al., Eur. Pat. Appl. (1982), EP 47011A1 19820310, 1982). Subsequently, DCPIB has been used as a selective VRAC/I_{Cl,SWELL} inhibitor (Decher et al., Br J Pharmacol, 134, 1467-1479, 2001; Zhang et al., Nat Cell Biol, 19, 504-517, 2017; Kang et al., Nat Commun, 9, 367, 2018) (FIG. 2f), binding within the pore of the SWELL1-LRRC8 hexamer at a constriction point located at arginine 103 (R103; FIG. 2e)(Deneka et al., Nature, 2018; Kasuya et al., Nat Struct Mol Biol, 25, 797-804, 2018; Kefauver et al., Elife, 7, 2018; Kern et al., Elife, 8, 2019), with an IC₅₀ of ˜2-4 μM (Decher et al., Br J Pharmacol, 134, 1467-1479, 2001; Best et al., Eur J Pharmacol, 489, 13-19, 2004). In experiments designed to pharmacologically inhibit VRAC/I_{Cl,SWELL} in signaling studies, we noted that DCPIB, which we here re-name Smod1, increases SWELL1 protein expression in 3T3-F442 preadipocytes β-fold) and adipocytes (1.5-fold; FIG. 2g-i) when applied for 96 hours, associated with enhanced insulin-stimulated pAKT2 (FIG. 2g-h&j), and insulin-stimulated pAS160 (FIG. 2k). These Smod1-mediated effects on insulin-AKT2-AS160 signaling are absent in SWELL1 KO 3T3-F442A adipocytes, consistent with an on-target, SWELL1-mediated mechanism of action (FIG. 2h&j).

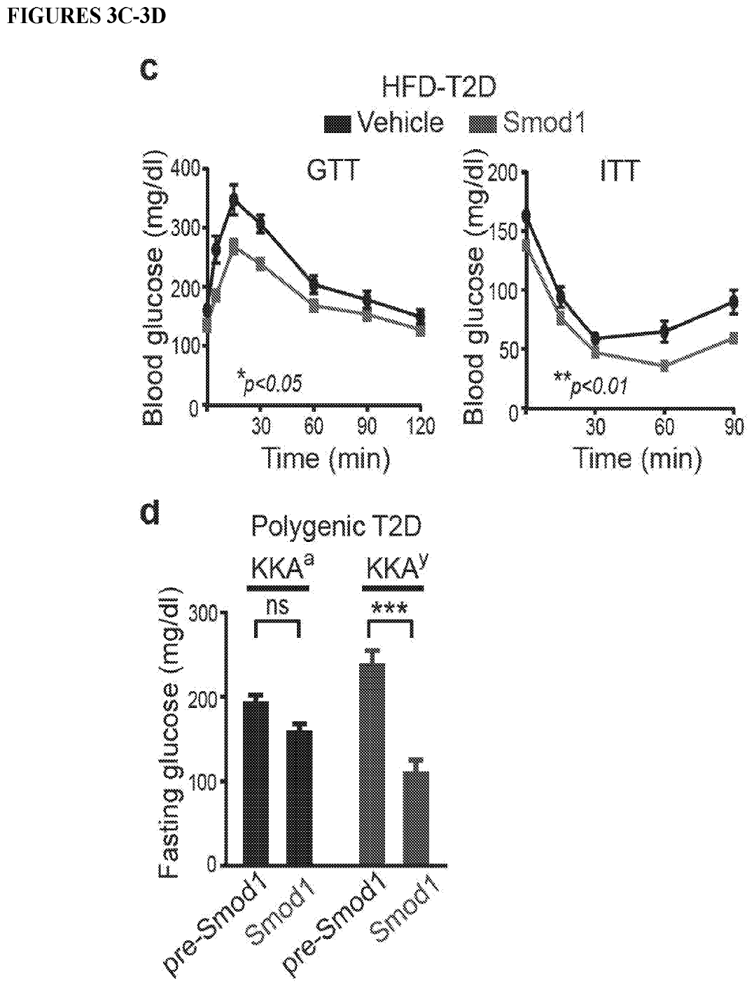

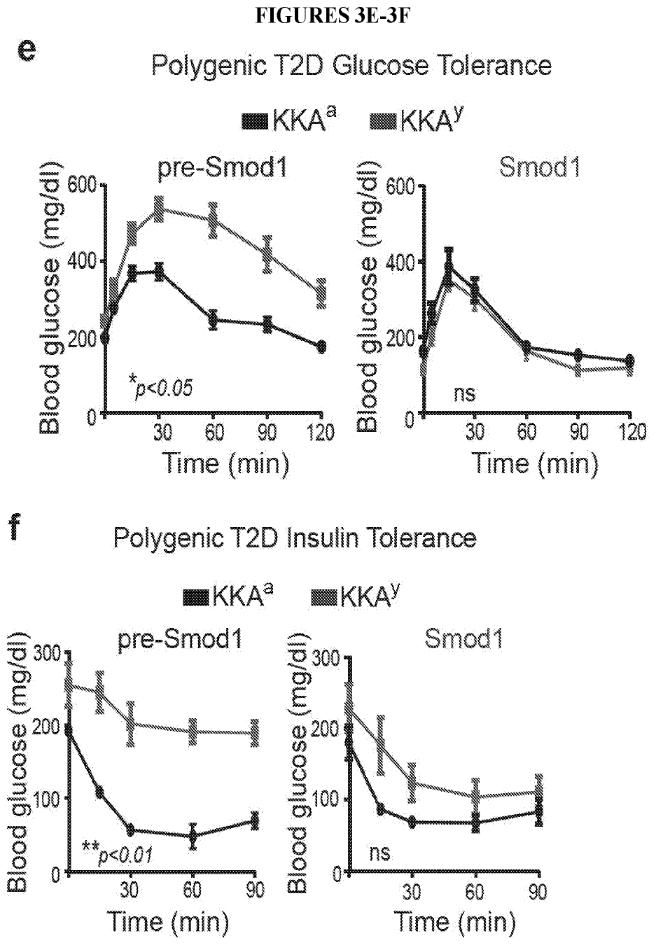

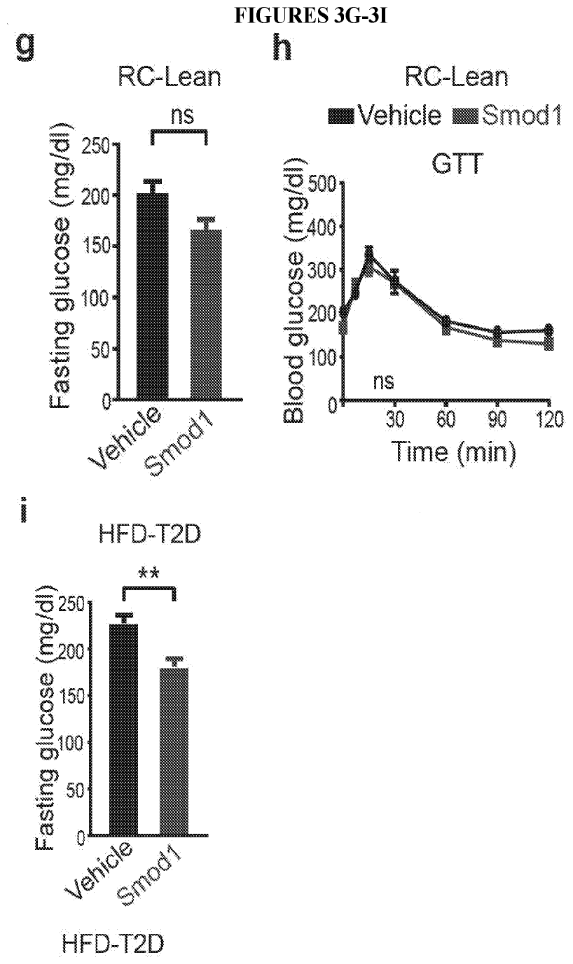

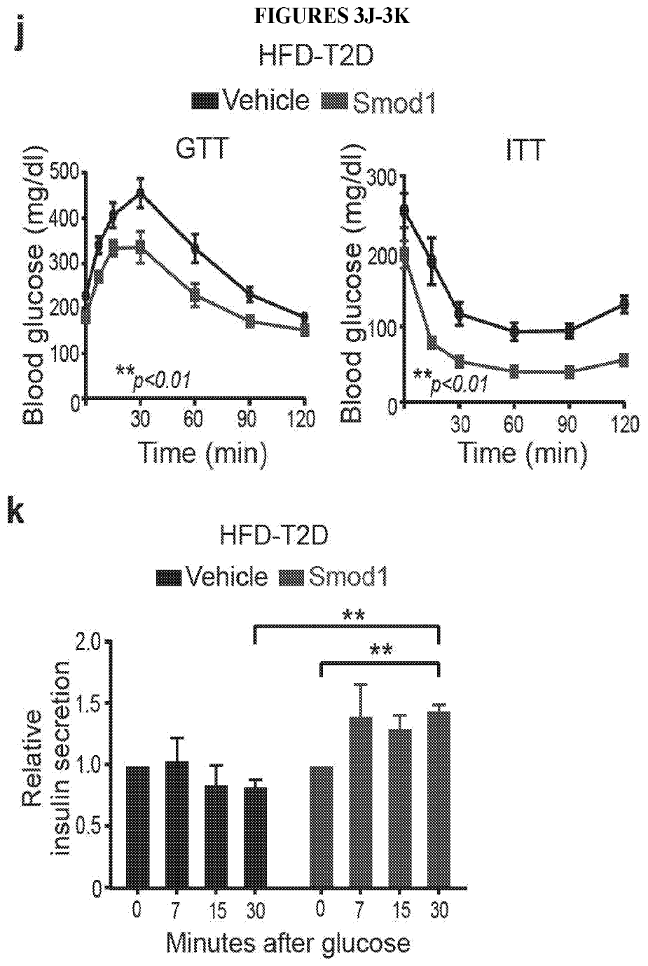

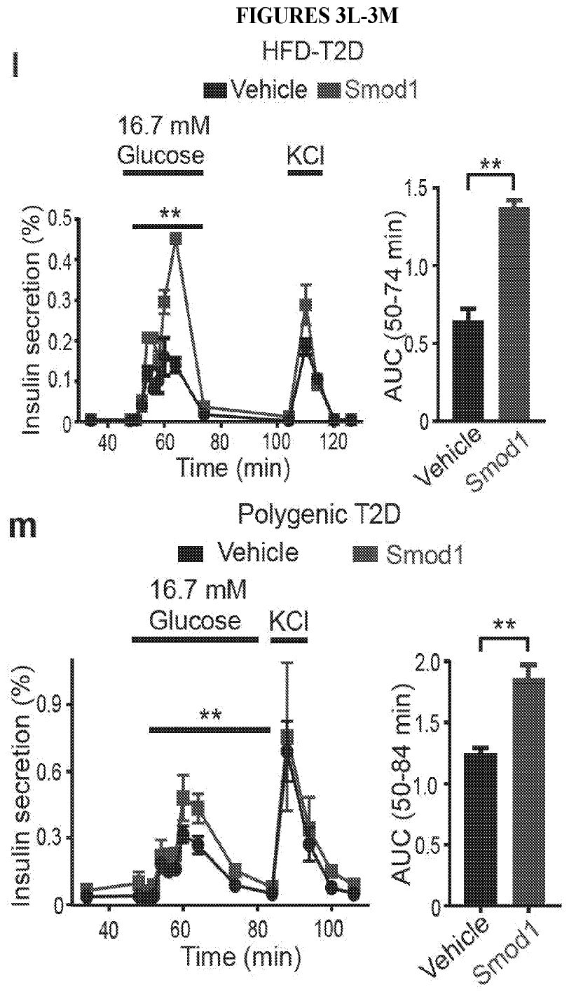

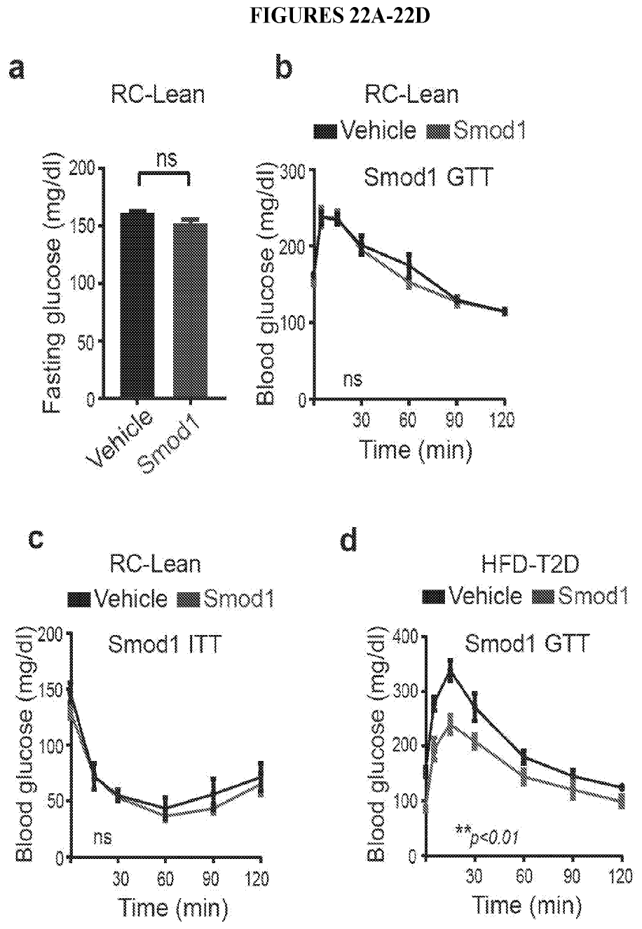

To determine if the observed effects of Smod1 on cultured adipocytes have activity on insulin signaling and glucose homeostasis in vivo we treated two T2D mouse models: obese, HFD-fed mice and the polygenic KKA^{y} mouse model with Smod1 (5 mg/kg i.p. for 4-10 days). In vivo, Smod1 augments SWELL1 expression 2.3-fold in adipose tissue of HFD-fed T2D mice (FIG. 3a). Similarly, Smod1 increases KKA^{y} adipose SWELL1 expression to levels comparable to both non-T2D C57/B6 mice and to the parental KKA^{a} parental strain (FIG. 3b). This restoration of SWELL1 expression is associated with normalized fasting glucose (FG), glucose tolerance (GTT), and markedly improved insulin-tolerance (ITT) in both HFD-induced T2D mice and in the polygenic Type 2 diabetes KKA^{y} model (FIG. 3c-f). Remarkably, treating the control KKA^{a} parental strain with Smod1 at the same treatment dose (5 mg/kg×4-10 days) does not cause hypoglycemia, nor does it alter glucose and insulin tolerance (FIG. 3d-f). Similarly, lean, non-T2D, glucose-tolerant mice treated with Smod1 have similar FG, GTT and ITT compared to vehicle treated mice (FIG. 3g&h and FIG. 22a-c, separate cohort of mice), however when re-treated with Smod1, after 16 weeks of HFD feeding, these HFD-induced T2D mice show marked improvements in FG (FIG. 3i), GTT and ITT (FIG. 3j). These data reveal that Smod1 is preferentially effective at normalizing glycemia in the setting of T2D, while lacking significant activity in non-T2D mice, and thus portend a low risk for inducing hypoglycemia. Moreover, Smod1 was well-tolerated during chronic injection protocols, with no overt signs of toxicity with daily injections for up to 8 weeks, despite remarkable effects on glucose tolerance (FIG. 22d).

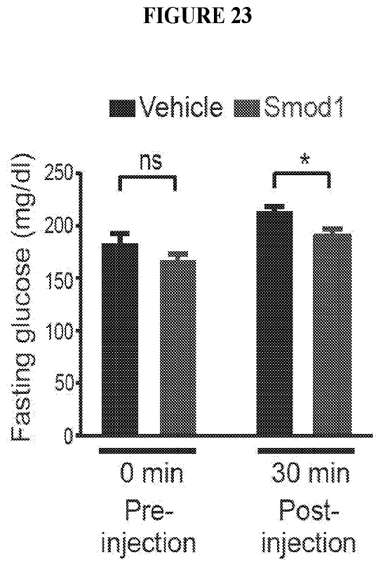

To examine the possible contribution of Smod1-mediated enhancements in insulin secretion from pancreatic β-cells, we next measured glucose-stimulated insulin secretion (GSIS) in mice subjected to 21 weeks of HFD. We find that the impairments in glucose-stimulated insulin secretion (GSIS) classically observed with long-term HFD (21 weeks HFD) are significantly improved in Smod1-treated HFD mice based on serum insulin measurements (FIG. 3k) and perifusion GSIS from isolated islets (FIG. 3l), consistent with the predicted effect of SWELL1 induction in pancreatic β-cells (Kang et al., Nat Commun, 9, 367, 2018; Stuhlmann et al., Nat Commun, 9, 1974, 2018). Similar results are obtained in perfusion assays performed in Smod1 compared to vehicle treated KKA^{y} mice (FIG. 3m). In fact, Smod1-mediated inhibition of I_{Cl,SWELL} in pancreatic β-cells is predicted to potentially cause hyperglycemia if circulating levels are sufficient to significantly block depolarizing β-cell I_{Cl,SWELL} and impair stimulus-secretion (Best et al., Eur J Pharmacol, 489, 13-19, 2004). To examine this possibility, we measured random glucose 30 minutes after Smod1 injection and observed no evidence of exacerbated hyperglycemia (FIG. 23). Collectively, these data suggest that Smod1-mediated improvements in systemic glycemia in T2D occur via augmentation of both peripheral insulin sensitivity and 3-cell insulin secretion—the inverse phenotype to in vivo loss-of-function studies (Xie et al., Channels (Austin), 11(6): 673-677, 2017; Zhang et al., Nat Cell Biol, 19, 504-517, 2017; Kang et al., Nat Commun, 9, 367, 2018; Stuhlmann et al., Nat Commun, 9, 1974, 2018).

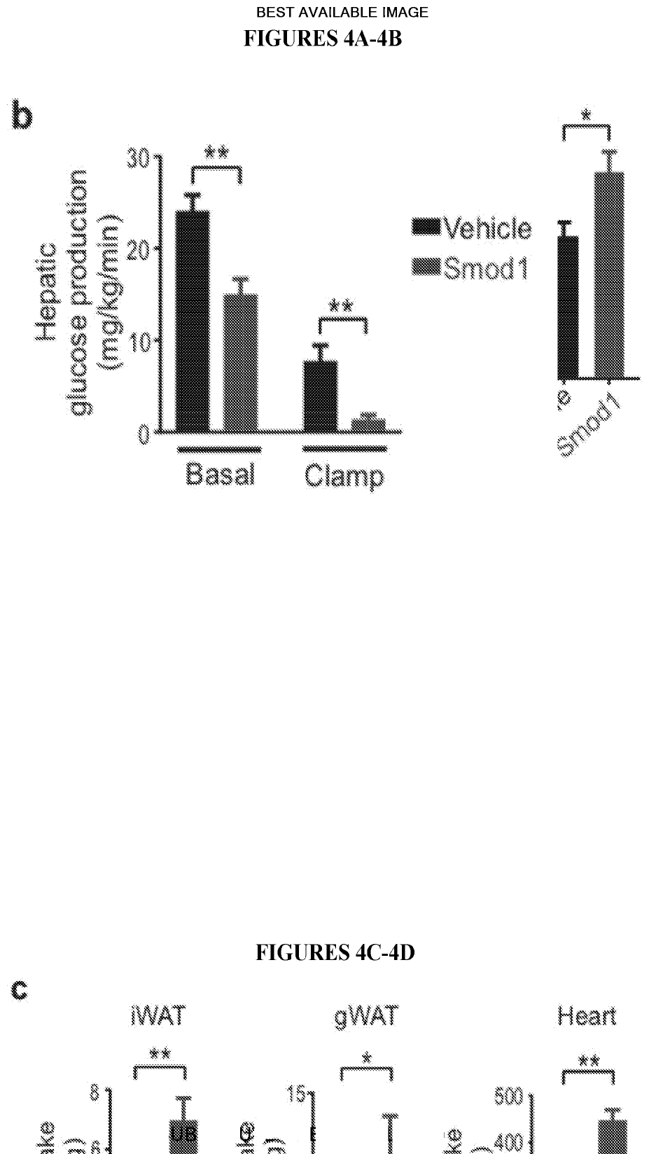

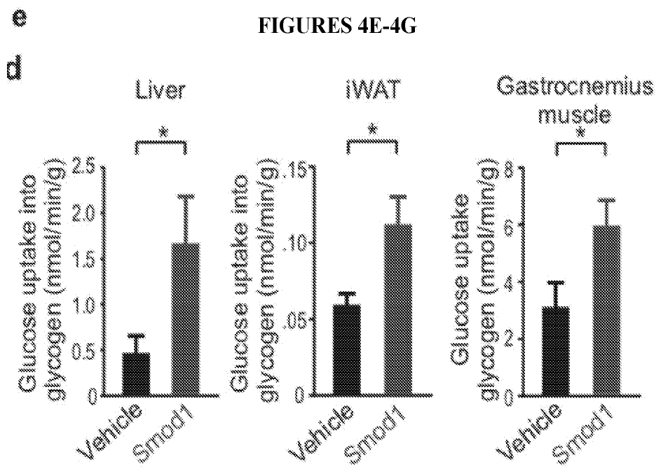

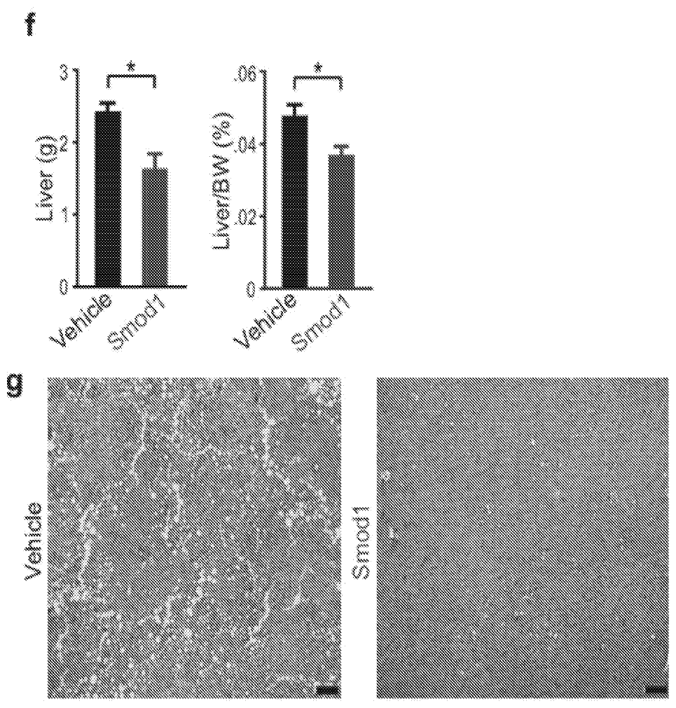

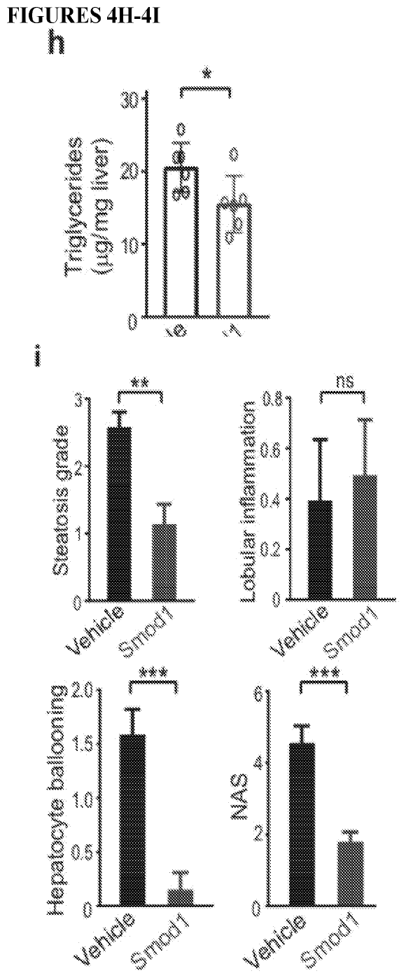

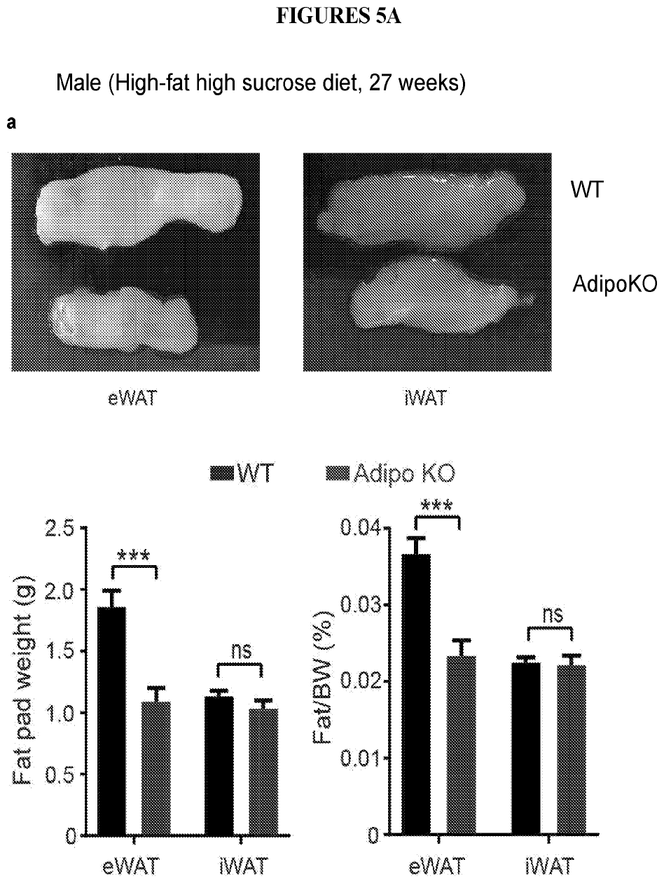

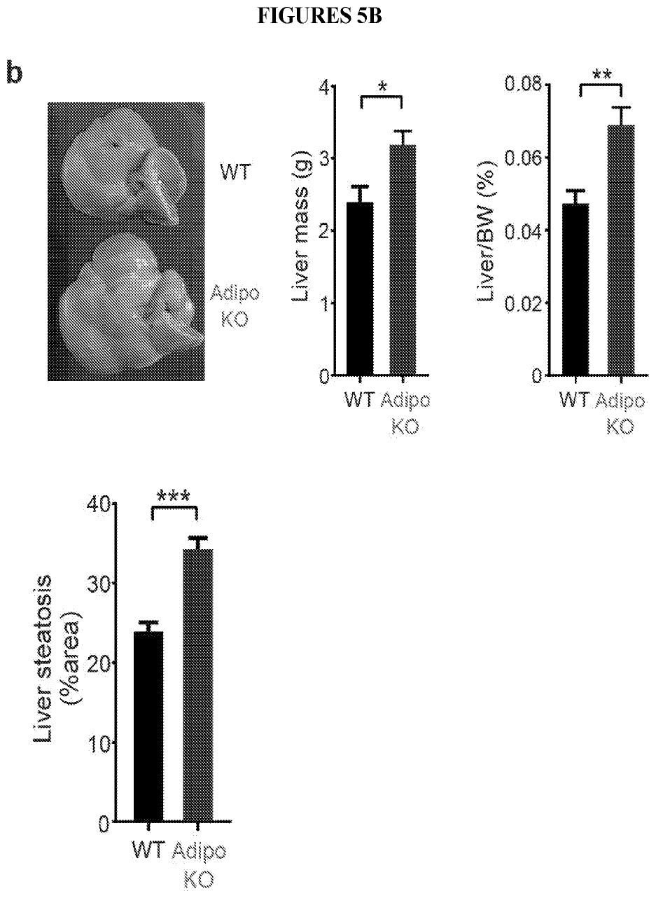

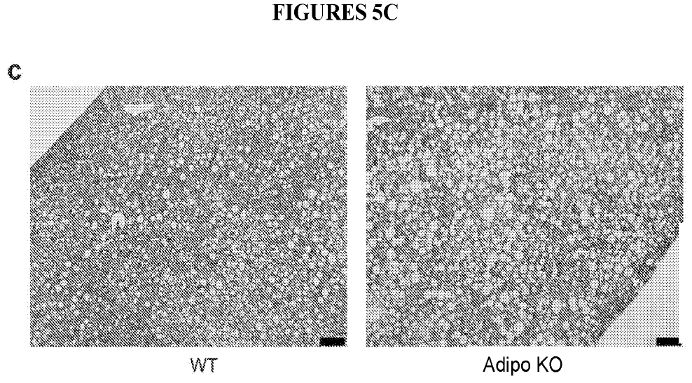

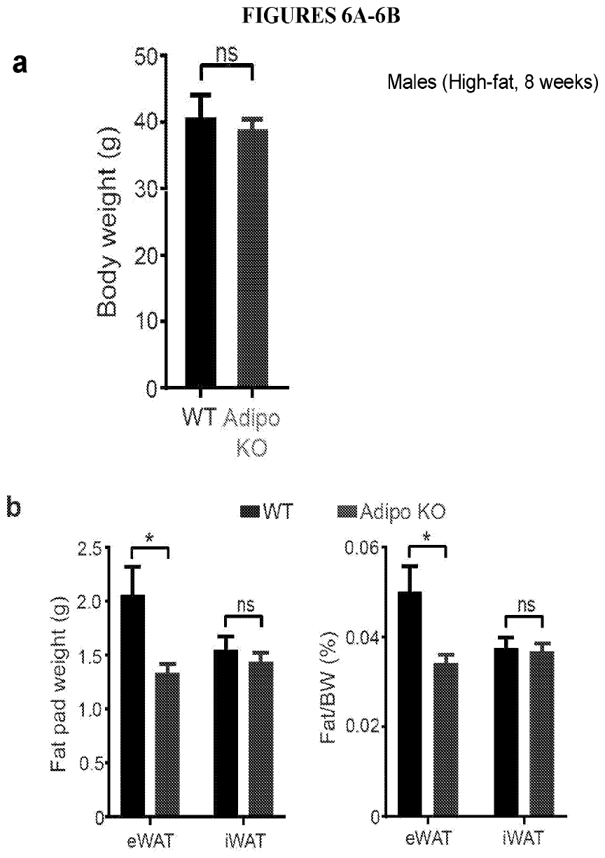

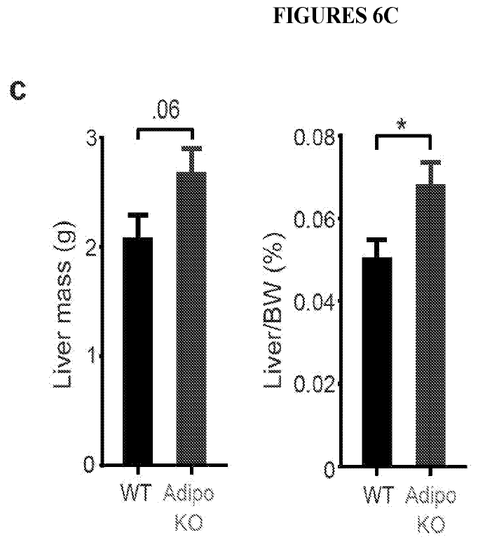

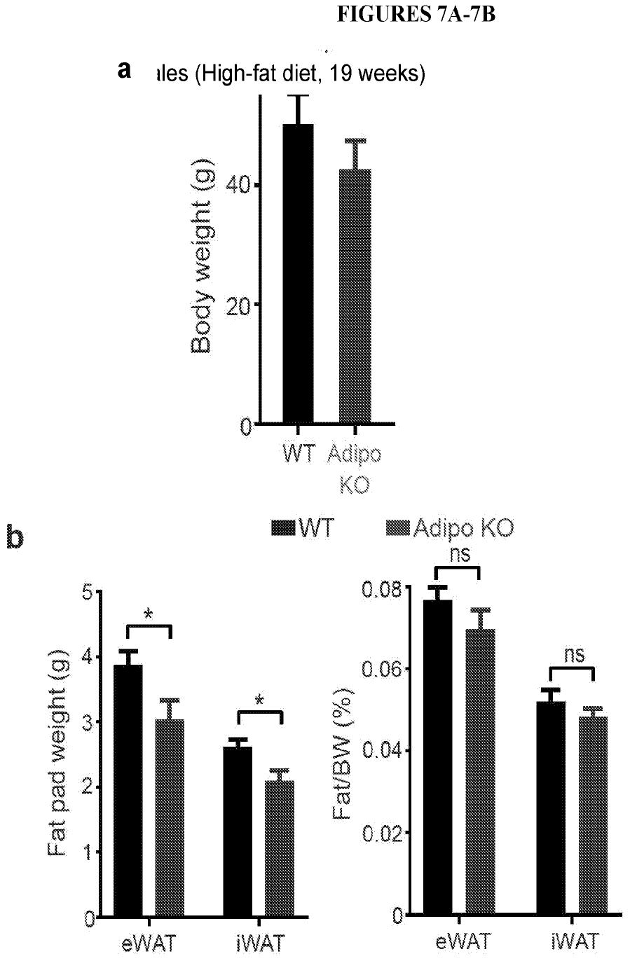

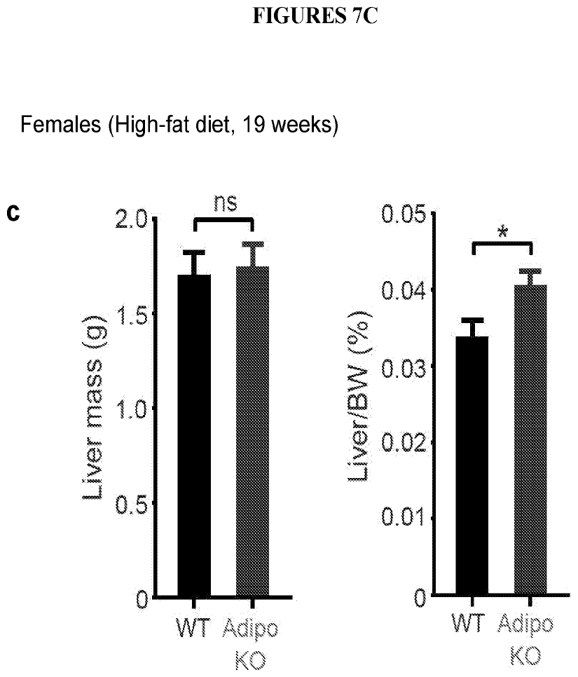

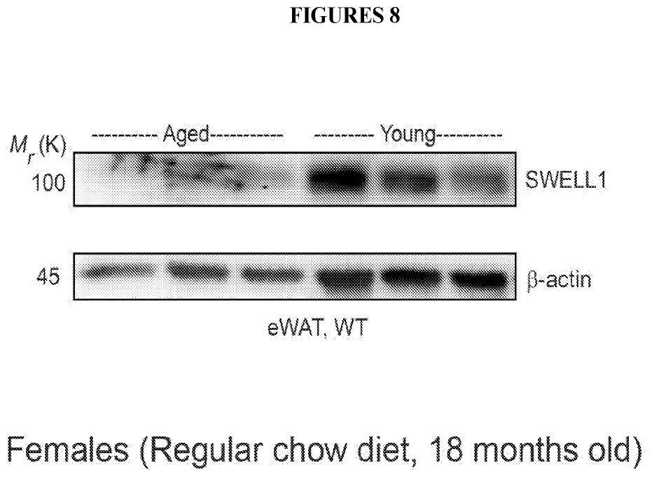

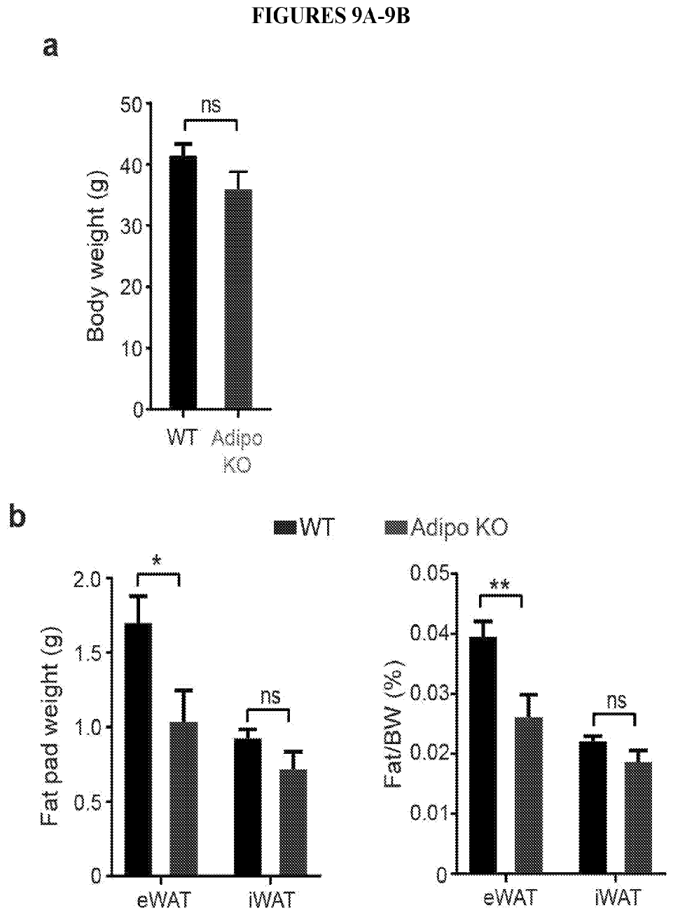

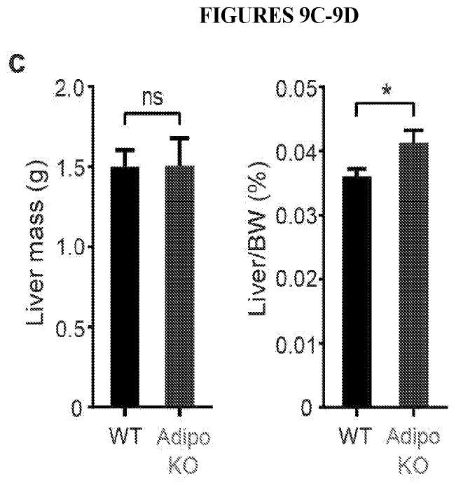

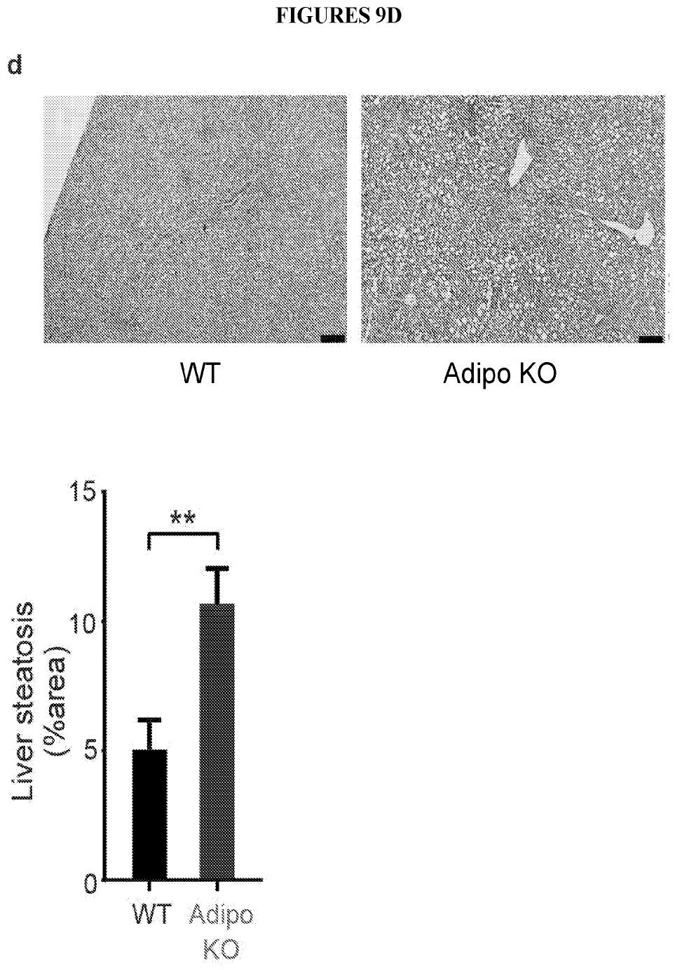

To more rigorously evaluate Smod1 effects on both insulin-sensitization and glucose metabolism in T2D mice we performed traced euglycemic hyperinsulinemic clamps, using both ³H-glucose and ¹⁴C-glucose, in KKA^{y} mice treated with Smod1 compared to vehicle. Smod1 treated KKA^{y} mice require a higher glucose-infusion rate (GIR) to maintain euglycemia as compared to vehicle (GIR,_{Smod1}=33.58±3.4 mg/kg/min and GIR,_{Veh}=23.23±2.4 mg/kg/min, p<0.05), consistent with enhanced systemic insulin-sensitivity (FIG. 4a). Hepatic glucose production from gluconeogenesis and/or glycogenolysis (Rₐ, rate of glucose appearance) is reduced ˜1.6-fold in Smod1-treated KKA^{y} mice at baseline (basal, FIG. 4b), and further suppressed 4.8-fold during the glucose/insulin infusion (clamp, FIG. 4b). These data reflect Smod1-mediated improvements in hepatic insulin-sensitivity. The mechanisms of the improvement in liver insulin-sensitivity are unknown, but could be tied to the effects of Smod1 on adipose tissue, for example reducing non-esterified fatty acid (NEFA) flux from adipose to liver due to enhanced adipose-insulin sensitivity and insulin-induced reductions in lipolysis.

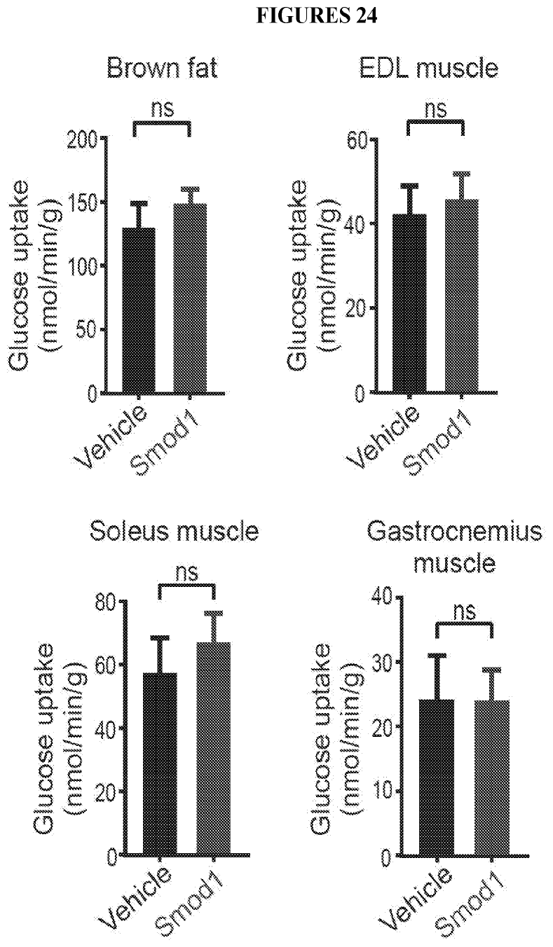

As Smod1-mediated SWELL1 induction is expected to enhance insulin-pAKT2-pAS160, GLUT4 translocation and glucose uptake (Zhang et al., Nat Cell Biol, 19, 504-517, 2017), we next measured tissue uptake of glucose determined by 2-deoxyglucose (2-DG) in adipose, myocardium and skeletal muscle. Smod1 enhanced insulin stimulated 2-DG uptake into inguinal white adipose tissue (iWAT-Smod1=7.0±0.78 nmol/min/g; iWAT-Veh=3.5±0.57 nmol/min/g, p<0.01), visceral white adipose tissue (gWAT-Smod1=11.99 2.75 nmol/min/g; gWAT-Veh=5.0±0.5 nmol/min/g, p<0.05), and myocardium (Heart-Smod1=451.3±24.63 nmol/min/g; Heart-Veh=318.15±23.48 nmol/min/g, p<0.01) (FIG. 4c), but not in brown fat, or skeletal muscle (FIG. 24). As adipocyte SWELL1 ablation markedly reduces insulin-pAKT2-pGSK30 regulated cellular glycogen content (Zhang et al., Nat Cell Biol, 19, 504-517, 2017), we next asked whether Smod1-mediated enhancements in SWELL1 expression may conversely increase glucose incorporation into tissue glycogen in the setting of T2D. Indeed, liver, adipose and skeletal muscle glucose-incorporation into glycogen is markedly increased in Smod1-treated mice (FIG. 4d), consistent with a SWELL1 mediated insulin-pAKT2-pGSK30-glycogen synthase gain-of-function.

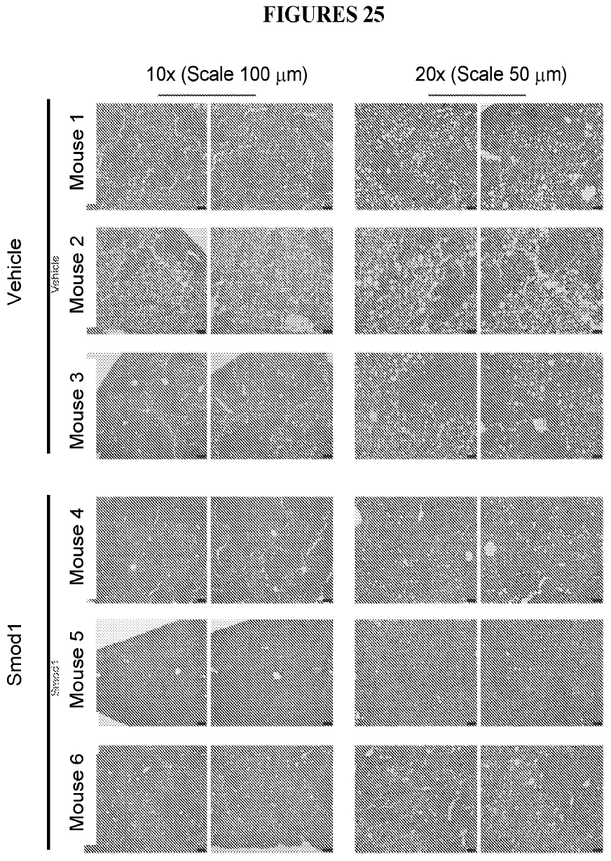

We next asked whether longer-term Smod1 treatment, and the associated sustained improvement in glucose homeostasis, may have salutary effects on non-alcoholic fatty liver disease (NAFLD), a common disease associated with metabolic syndrome for which there is currently no approved therapy. In a cohort of mice treated chronically and intermittently with Smod1 over the course of 21 weeks (FIG. 4e), we note a marked reduction in liver weights (FIG. 4f), in hepatic steatosis on histology (FIG. 4g and FIG. 25), and in liver triglycerides (FIG. 4h). The NAFLD activity score (NAS), which integrates hepatic steatosis, lobular inflammation, and hepatic ballooning (FIG. 4i) is also significantly improved in Smod1-treated mice compared to vehicle treated control mice. Taken together, these data reveal that Smod1 augments SWELL1 expression and SWELL1-mediated signaling to concomitantly enhance both systemic insulin-sensitivity and pancreatic j-cell insulin secretion to normalize systemic glycemia in T2D mouse models. This improved metabolic state abrogates ectopic lipid deposition and NAFLD that is associated with obesity and T2D.

Our current working model is that the transition from “compensated” obesity (pre-diabetes, normoglycemia) to “decompensated” obesity (T2D, hyperglycemia) reflects, among other things, a relative reduction in SWELL1 protein expression and signaling in peripheral insulin-sensitive tissues (Inoue et al., Am J Physiol Cell Physiol, 298, C900-909, 2010; Xie et al., Channels (Austin), 11(6): 673-677, 2017) and in pancreatic β-cells (Taneera et al., Cell Metab, 16, 122-134, 2012; Osei-Owusu et al., Curr Top Membr, 81, 177-203, 2018)-metabolically pheno-copying SWELL1-loss-of-function models (Xie et al., Channels (Austin), 11(6): 673-677, 2017; Zhang et al., Nat Cell Biol, 19, 504-517, 2017; Kang et al., Nat Commun, 9, 367, 2018; Stuhlmann et al., Nat Commun, 9, 1974, 2018). Therefore, we speculate that rectifying this SWELL1-deficient state may improve overall systemic glycemia via both insulin-sensitization and secretion mechanisms. Curiously, we discovered that Smod1/DCPIB, a selective I_{Cl,SWELL} inhibitor (Decher et al., Br J Pharmacol, 134, 1467-1479, 2001; Best et al., Eur J Pharmacol, 489, 13-19, 2004), unexpectedly induces SWELL1 protein, which in turn augments insulin-sensitivity and 3-cell insulin secretion—thereby normalizing systemic glycemia and mitigating NAFLD in T2D mice, without inducing hypoglycemia in non-T2D controls. While the Smod1 mechanism of action may appear somewhat paradoxical—an inhibitor that actually augments it's targets signaling activity—it is not unlike the mechanism of action of β-blockers in improving cardiac function in the setting of heart failure (Baker et al., Trends Pharmacol Sci, 32, 227-234, 2011). And just like the β-blocker story, fully elucidating the molecular mechanism of Smod1 action to explain these observations will require further work, since the detailed molecular pathways are likely more complex than can be possibly divined based on our current understanding of this very nascent field (Qiu et al., Cell, 157, 447-458, 2014; Voss et al., Science, 344, 634-638, 2014). One intriguing molecular mechanism may relate to the requirement for SWELL1 to form a macromolecular signaling complex that includes heterohexamers of SWELL1 and LRRC8b-e, with stoichiometries that probably vary from tissue to tissue. We propose that forming stable SWELL1 signaling complexes may be challenging, and thus result in a proportion of mis-assembled complexes that are subject to protein degradation. This is expected to be further exacerbated under conditions of elevated endoplasmic reticulum (ER) stress associated with T2D states (Back et al., Exp Diabetes Res, 2012, 618396, 2012; Back et al., Annu Rev Biochem, 81, 767-793, 2012; Cnop et al., Trends Mol Med, 18, 59-68, 2012), and may account for the reductions in SWELL1 protein and SWELL1-mediated I_{Cl,SWELL} observed in T2D. In this case, small molecules that stabilize formation of the SWELL1-LRRC8 complex, such as Smod1/DCPIB (Kern et al., Elife, 8, 2019), may serve to augment SWELL1 protein and the number of active SWELL1-LRRC8 signaling complexes by enhancing the passage of SWELL1-LRRC8 heteromers through the ER and Golgi apparatus, especially in the setting of T2D associated ER stress. Indeed, the recent structure of the LRRC8a/SWELL1 homo-hexamer with DCPIB bound in the pore implies that DCPIB assists in stabilizing the SWELL1 hexamer for cryo-EM imaging in lipid nanodiscs (Kern et al., Elife, 8, 2019), and suggests that DCPIB-like small molecules may also stabilize SWELL1-LRRC8 complexes in vivo. Moreover, the concept of small molecule inhibitors acting as therapeutic molecular chaperones (Buchner, J Biol Chem, 294, 2074-2075, 2019) to support the folding, assembly and trafficking of proteins (including ion channels) has been demonstrated for Niemann-Pick C disease (Pipalia et al., Proc Natl Acad Sci USA, 108, 5620-5625, 2011; Pipalia et al., J Lipid Res, 58, 695-708, 2017), congenital hyperinsulinism (SUR1-K_{ATP} channel mutants)(Pratt et al., J Biol Chem, 284, 7951-7959, 2009; Chen et al., J Biol Chem, 288, 20942-20954, 2013; Martin et al., J Biol Chem, 291, 21971-21983, 2016), and Cystic Fibrosis (CFTR mutants)(Loo et al., Biochem J, 413, 29-36, 2008; Pedemonte et al., Front Pharmacol, 3, 175, 2012), thus providing precedent for our hypothesized mechanism of action.

The current study provides an initial proof-of-concept for pharmacological induction of SWELL1 signaling using SWELL1 modulators (Smods) to treat metabolic syndrome at multiple homeostatic nodes, including adipose, liver, and pancreatic β-cell. Hence, Smod1 may represent a tool compound from which a novel drug class may be derived to treat both T2D, metabolic syndrome, and associated diseases such as NAFLD, the latter of which currently lacks therapeutic options, and thus represents a significant unmet need.

### Methods

Patients Human islets and adipocytes were obtained and cultured as described previously (Zhang et al., Nat Cell Biol, 19, 504-517, 2017; Kang et al., Nature Communications, 9, 367, 2018). The Patients involved in the study were anonymous and information such as gender, age, HbA1c, glucose levels and BMI only were available to the research team. The study was approved and carried out as per the guidelines of the University of Iowa Institutional Review Board (IRB).
Animals All experimental procedures involving mice were approved by the Institutional Animal Care and Use Committee of the University of Iowa and Washington University at St. Louis. All C57BL/6 mice involved in study were purchased from Charles River Labs. Both KK.Cg-Ay/J (KKA^{y}) and KK.Cg-Aa/J (KKA^{a}) mice involved in study were gender and age-matched mice obtained from Jackson Labs (Stock No: 002468) and bred up for experiments. The mice were fed ad libitum with either regular chow (RC) or high-fat diet (Research Diets, Inc., 60 kcal % fat) with free access to water and housed in a light-, temperature- and humidity-controlled room. For high-fat diet (HFD) studies, only male mice were used and were started on HFD regimen at the age of 6-9 weeks. For all experiments involving KKA^{y} and KKA^{a} mice, both males and females were used at approximately 50/50 ratio. In all experiments involving mice, investigators were kept blinded both during the experiments and subsequent analysis.
Smod1 (DCPIB) treatment Smod1 (DCPIB, Tocris, #D1540), was dissolved in Kolliphor® EL (Sigma, #C5135). Either vehicle (Kolliphor® EL) or Smod1 (5 mg/kg of body weight/day) were injected (intraperitoneal, i.p.) using 1 cc syringe/26G/12 inch needle daily for 4-10 days, and in one experiment, daily for 8 weeks.
Adenovirus Adenovirus type 5 with Ad5-RIP2-GFP (4.1×10¹⁰ PFU/ml) and Ad5-CAG-LoxP-stop-LoxP-3× Flag-SWELL1 (1×10¹⁰ PFU/ml) were obtained from Vector Biolabs. Adenovirus type 5 with Ad5-CMV-Cre-wt-IRES-eGFP (8×10¹⁰ PFU/ml) was obtained from the University of Iowa Viral Vector Core.
Cell culture Wildtype (WT) and SWELL1 knockout (KO) 3T3-F442A (Sigma-Aldrich) cells were cultured and differentiated as described previously (Zhang et al., Nat Cell Biol, 19, 504-517, 2017). Preadipocytes were maintained in 90% DMEM (25 mM D-Glucose and 4 mM L-Glutamine) containing 10% fetal bovine serum (FBS) and 100 IU penicillin and 100 μg/ml streptomycin on collagen-coated (rat tail type-I collagen, Corning) plates. Upon reaching confluency, the cells were differentiated in the above-mentioned media supplemented with 5 μg/ml insulin (Cell Applications) and replenished every other day with the differentiation media. For insulin signaling studies on WT and KO adipocytes with or without SWELL1 overexpression (O/E), the cells were differentiated for 10 days and transduced with Ad5-CAG-LoxP-stop-LoxP-3× Flag-SWELL1 virus (MOI 12) on day 11 in 2% FBS containing differentiation medium. To induce the overexpression, Ad5-CMV-Cre-wt-IRES-eGFP (MOI 12) was added on day 13 in 2% FBS containing differentiation medium. The cells were then switched to 10% FBS containing differentiation medium from day 15 to 17. On day 18, the cells were starved in serum free media for 6 h and stimulated with 0 and 10 nM insulin for either 5 or 15 min. Either Ad5-CAG-LoxP-stop-LoxP-3× Flag-SWELL1 or Ad5-CMV-Cre-wt-IRES-eGFP virus transduced cells alone were used as controls. Based on GFP fluorescence, viral transduction efficiency was ˜90%.

For Smod1 (DCPIB) mediated induction of SWELL1 and insulin signaling studies in 3T3-F442A preadipocytes, the cells were incubated with either vehicle (DMSO) or 10 μM Smod1 for 96 h. The cells were serum starved for 6 h (+DMSO or Smod1) and stimulated with 0, 3 and 10 nM insulin containing media (−DMSO or Smod1), as described above for 15 mins and the lysates were collected. In the case of 3T3-F442A adipocytes, the WT and KO cells were treated with either vehicle (DMSO) or 10 μM Smod1 following 7-11 days of differentiation for 96 h and then stimulated with 0 and 10 nM insulin for 15 min, as described above. Human embryonic kidney (HEK) cells were cultured and maintained in 90% DMEM (25 mM D-Glucose and 4 mM L-Glutamine) containing 10% fetal bovine serum (FBS) and 100 IU penicillin and 100 μg/ml streptomycin media.

Electrophysiology Patch-clamp recordings of β-cells and mature adipocytes were performed as described previously (Zhang et al., Nat Cell Biol, 19, 504-517, 2017; Kang et al., Nature Communications, 9, 367, 2018). 3T3-F442A WT and KO preadipocytes were prepared as described in the Cell culture section above. For SWELL1 overexpression recordings, preadipocytes were first transduced with Ad5-CAG-LoxP-stop-LoxP-3× Flag-SWELL1 (MOI 12) in 2% FBS culture medium for two days and then overexpression induced by adding Ad5-CMV-Cre-wt-IRES-eGFP (MOI 10-12) in 2% FBS culture medium for two more days and changed to 10% FBS containing culture media and were selected based on GFP expression (˜2-3 days). For 3-cell recordings, islets were transduced with Ad-RIP2-GFP and then dispersed after 48-72 hours for patch-clamp experiments. GFP+ cells marked j-cells selected for patch-clamp recordings. Recordings were measured using Axopatch 200B amplifier paired to a Digidata 1550 digitizer using pClamp 10.4 software. The extracellular buffer composition for hypotonic stimulation contains 90 mM NaCl, 2 mM CsCl, 1 mM MgCl₂, 1 mM CaCl₂), 10 mM HEPES, 10 mM Mannitol, pH 7.4 with NaOH (210 mOsm/kg). The extracellular isotonic buffer composition is same as above except for Mannitol concentration of 110 mM (300 mOsm/kg). The composition of intracellular buffer is 120 mM L-aspartic acid, 20 mM CsCl, 1 mM MgCl₂, 5 mM EGTA, 10 mM HEPES, 5 mM MgATP, 120 mM CsOH, 0.1 mM GTP, pH 7.2 with CsOH. All recordings were carried out at room temperature (RT) with j-cells and 3T3-F442A cells performed in whole-cell configuration and human adipocytes in perforated-patch configuration, as previously (Zhang et al., Nat Cell Biol, 19, 504-517, 2017; Kang et al., Nature Communications, 9, 367, 2018).
Western blot Cells were washed twice in ice-cold phosphate buffer saline and lysed in RIPA buffer (150 mM Nacl, 20 mM HEPES, 1% NP-40, 5 mM EDTA, pH 7.4) with proteinase/phosphatase inhibitors (Roche). The cell lysate was further sonicated in 10 sec cycle intervals for 2-3 times and centrifuged at 14000 rpm for 20 min at 4° C. The supernatant was collected and further estimated for protein concentration using DC protein assay kit (Bio-Rad). Fat tissues were homogenized and suspended in RIPA buffer with inhibitors in similar fashion as described above. Protein samples were further prepared by boiling in 4× laemmli buffer. Approximately 10-20 μg of total protein was loaded in 4-15% gradient gel (Bio-Rad) for separation and protein transfer was carried out onto the PVDF membranes (Bio-Rad). Membranes were blocked in 5% BSA (or 5% milk for SWELL1) in TBST buffer (0.2 M Tris, 1.37 M NaCl, 0.2% Tween-20, pH 7.4) for 1 h and incubated with appropriate primary antibodies (5% BSA or milk) overnight at 4° C. The membranes were further washed in TBST buffer before adding secondary antibody (Bio-Rad, Goat-anti-rabbit, #170-6515) in 1% BSA (or 1% milk for SWELL1) in TBST buffer for 1 h at RT. The signals were developed by chemiluminescence (Pierce) and visualized using a Chemidoc imaging system (Biorad). The images were further analyzed for band intensities using ImageJ software. Following primary antibodies were used: anti-phospho-AKT2 (#8599s), anti-AKT2 (#3063s), anti-phospho-AS160 (#4288s), anti-AS160 (#2670s) and anti-3-Actin (#8457s) from Cell Signaling; anti-SWELL1 as previously described.
Immunofluorescence 3T3-F442A preadipocytes (WT, KO) and differentiated adipocytes without or with SWELL1 overexpression (WT+SWELL1 O/E, KO+SWELL1 O/E) were prepared as described in the Cell culture section. Cells were then plated on collagen coated cover slips and fixed in ice-cold acetone for 15 min at −20° C. The cells were then washed four times with 1×PBS and permeabilized with 0.1% Triton X-100 in 1×PBS for 5 min at RT and subsequently blocked with 5% normal goat serum for 1 h at RT. Either anti-SWELL1 (1:400) or anti-Flag (1:1500, Sigma #F3165) antibody were added to the cells and incubated overnight at 4° C. The cells were then washed three times (1×PBS) prior and post to the addition of 1:1000 Alexa Flour 488/568 secondary antibody (anti-rabbit, #A11034 or anti-mouse, #A11004) for 1 h at RT. Cells were counterstained with nuclear TO-PRO-3 (Life Technologies, #T3605) staining for 20 min followed by three washes with 1×PBS. Coverslips were further mounted on slides with ProLong Diamond anti-fading media. All images were captured using Zeiss LSM700/LSM510 confocal microscope with 63× objective (NA 1.4).
Metabolic phenotyping Mice were fasted for 6 h prior to glucose tolerance tests (GTT). Baseline glucose levels at 0 min timepoint (fasting glucose, FG) were measured from blood sample collected from tail snipping using glucometer (Bayer Healthcare LLC). Either 1 g or 0.75 g D-Glucose/kg body weight were injected (i.p.) for lean or HFD mice, respectively and glucose levels were measured at 7, 15, 30, 60, 90 and 120 min timepoints after injection. For insulin tolerance tests (ITTs), the mice were fasted for 4 h. Similar to GTTs, the baseline blood glucose levels were measured at 0 min timepoint and 15, 30, 60, 90 and 120 min timepoints post-injection (i.p.) of insulin (HumulinR, IU/kg body weight for lean mice or 1.25 U/kg body weight for HFD mice). GTTs or ITTs with Smod1 (or vehicle) treated groups were performed approximately 24 hours after the last injection. For insulin secretion assay, the vehicle or Smod1 treated HFD mice were fasted for 6 h and injected (i.p.) with 0.75 g D-Glucose/kg body weight and blood samples were collected at 0, 7, 15 and 30 min time points in microvette capillary tubes (SARSTEDT, #16.444) and centrifuged at 5000 rpm for 20 min at 4° C. The collected plasma was then measured for insulin content by using Ultra Sensitive Mouse Insulin ELISA Kit (Crystal Chem, #90080). All mice and treatment groups were assessed blindly while performing experiments.
Murine islet isolation and perifusion assay For patch-clamp studies involving primary mouse β-cells, the mice were anesthesized by injecting Avertin (0.0125 g/ml in H₂O) followed by cervical dislocation. HFD or polygenic KKAy mice treated with either vehicle or Smod1 were anesthesized with 14% isoflurane followed by cervical dislocation. Islets were further isolated as described previously (Kang et al., Nature Communications, 9, 367, 2018). The perifusion of islets were performed using a PERI4-02 from Biorep Technologies. For each experiment, around 50 freshly isolated islets (all from the same isolation batch) were handpicked to match size of islets across the samples and loaded into the polycarbonate perifusion chamber between two layers of polyacrylamide-microbead slurry (Bio-Gel P-4, BioRad) by the same experienced operator. Perifusion buffer contained (in mM): 120 NaCl, 24 NaHCO₃, 4.8 KCl, 2.5 CaCl₂), 1.2 MgSO₄, 10 HEPES, 2.8 glucose, 27.2 mannitol, 0.25% w/v bovine serum albumin, pH 7.4 with NaOH (300 mOsm/kg). Perifusion buffer kept at 37° C. was circulated at 120 l/min. After 48 min of washing with 2.8 mM glucose solution for stabilization, islets were stimulated with the following sequence: 16 min of 16.7 mM glucose, 40 min of 2.8 mM glucose, 10 min of 30 mM KCl, and 12 min of 2.8 mM glucose. Osmolarity was matched by adjusting mannitol concentration when preparing solution containing 16.7 mM glucose. Serial samples were collected either every 1 or 2 min into 96 wells kept at 4° C. Insulin concentrations were further determined using commercially available ELISA kit (Mercodia). The area under the curve (AUC) for the high-glucose induced insulin release was calculated for time points between 50 to 74/84 min. At the completion of the experiments, islets were further lysed by addition of RIPA buffer and the amount of insulin was detected by ELISA.
Hyperinsulinemic euglycemic glucose clamp Sterile silicone catheters (Dow-Corning) were placed into the jugular vein of mice under isoflurane anesthesia. Placed catheter was flushed with 200 U/mL heparin in saline and the free end of the catheter was directed subcutaneously via a blunted 14-gauge sterile needle and connected to a small tubing device that exited through the back of the animal. Mice were allowed to recover from surgery for 3 days, then received IP injections of vehicle or Smod1 (5 mg/kg) for 4 days. Hyperinsulinemic euglycemic clamps were performed on day 8 post-surgery on unrestrained, conscious mice as described elsewhere (Kim et al., J Clin Invest, 105, 1791-1797, 2000; Ayala et al., J Vis Exp, 2011), with some modifications. Mice were fasted for 6 h at which time insulin and glucose infusion were initiated (time 0). At 80 min prior to time 0 basal sampling was conducted, where whole-body glucose flux was traced by infusion of 0.05 μCi/min D-[3-³H]-glucose (Perkin Elmer), after a priming 5 μCi bolus for 1 minute. After the basal period, starting at time 0 D-[3-³H]-glucose was continuously infused at the 0.2 μCi/min rate and the infusion of insulin (Humulin, Eli Lilly) was initiated with a bolus of 80 mU/kg/min then followed by continuous infusion of insulin at the dose of 8 mU/kg/min throughout the assay. Fifty percent dextrose (Hospira) was infused at a variable rates (GIR) starting at the same time as the initiation of insulin infusion to maintain euglycemia at the targeted level of 150 mg/dL (8.1 mM). Blood glucose (BG) measurements were taken every ten minutes via tail vein sampling using Contour glucometer (Bayer). After mouse reached stable BG and GIR (typically, after 75 minutes since starting the insulin infusion; for some mice, a longer time was required to achieve steady state) a single bolus of 12 μCi of [1-¹⁴C]-2-deoxy-D-glucose (Perkin Elmer) in 96 μl of saline was administered. Plasma samples (collected from centrifuged blood) for determination of tracers enrichment, glucose level and insulin concentration were obtained at times −80, −20, −10, 0, and every 10 min starting at 80 min post-insulin (5 min. after [1-¹⁴C]-2-deoxy-D-glucose bolus was administered) until the conclusion of the assay at 140 min. Tissue samples were then collected from mice under isofluorane anaesthesia from organs of interest (e.g., liver, heart, kidney, white adipose tissue, brown adipose tissue, gastrocnemius, soleus etc.) for determination of 1-¹⁴C]-2-deoxy-D-glucose tracer uptake.

Plasma and tissue samples were processed as described previously (Ayala et al., J Vis Exp, 2011). Briefly, plasma samples were deproteinized with Ba(OH)₂ and ZnSO₄ and dried to eliminate tritiated water. The glucose turnover rate (mg/kg-min) was calculated as the rate of tracer infusion (dpm/min) divided by the corrected plasma glucose specific activity (dpm/mg) per kg body weight of the mouse. Fluctuations from steady state were accounted for by use of Steele's model. Plasma glucose was measured using Analox GMD9 system (Analox Technologies).

Tissue samples (˜30 mg each) were homogenized in 750 μl of 0.5% perchloric acid, neutralized with 10 M KOH and centrifuged. The supernatant was then used for first measuring the abundance of total [1-¹⁴C] signal (derived from both 1-¹⁴C-2-deoxy-D-glucose, 1-¹⁴C-2-deoxy-D-glucose 6 phosphate) and, following a precipitation step with 0.3 N Ba(OH)₃ and 0.3 N ZnSO₄, for the measuring of non-phosphorylated 1-¹⁴C-2-deoxy-D-glucose.

Glycogen was isolated by ethanol precipitation from 30% KOH tissue lysates, as described (Shiota, Animal Models in Diabetes Research, 229-253, 2012).

Insulin level in plasma at TO and T140 were measured using a Stellux ELISA rodent insulin kit (Alpco).

Liver isolation, triglycerides and histology HFD mice treated with either vehicle or Smod1 were anesthesized with 14% isoflurane followed by cervical dislocation. Gross liver weights were measured and identical sections from right medial lobe of liver were dissected for further examinations. Total triglyceride content was determined by homogenizing 10-50 mg of tissue in 1.5 ml of chloroform:methanol (2:1 v/v) and centrifuged at 12000 rpm for 10 mins at 4° C. An aliquot, 20 ul, was evaporated in a 1.5 ml microcentrifuge tube for 30 mins. Triglyceride content was determined by adding 100 μl of Infinity Triglyceride Reagent (Fisher Scientific) to the dried sample followed by 30 min incubation at RT. The samples were then transferred to a 96 well plate along with standards (0-2000 mg/dl) and absorbance was measured at 540 nm and the final concentration was determined by normalizing to tissue weight. For histological examination, liver sections were fixed in 10% zinc formalin and paraffin embedded for sectioning. Haematoxylin and eosin (H&E) stained sections were then assessed for steatosis grade, lobular inflammation and hepatocyte ballooning for non-alcoholic fatty liver disease (NAFLD) scoring as described (Kleiner et al., Hepatology, 41, 1313-1321, 2005; Liang et al., PLoS One, 9, e115922, 2014; Rauckhorst et al., Mol Metab, 6, 1468-1479, 2017).

Statistical analysis Standard unpaired or paired two-tailed Student's t-test were performed while comparing two groups. For GTTs and ITTs, 2-way analysis of variance (Anova) was used. A p-value less than 0.05 was considered statistically significant. *, ** and *** represents a p-value less than 0.05, 0.01 and 0.001 respectively. All data are represented as mean±SEM. All statistical analysis are indicated in the respective legends.

### Example 2

The endothelium responds to various chemical and mechanical factors in regulating vascular tone, angiogenesis, systemic arterial and/or pulmonary arterial blood pressure and blood flow. The endothelial volume regulatory anion channel (eVRAC) has been proposed to be mechano-sensitive, to activate in response to fluid flow/hydrostatic pressure and putatively regulate vascular reactivity and angiogenesis. Here we show that the Leucine Rich Repeat Containing Protein 8a, LRRC8a (SWELL1) functionally encodes eVRAC in human umbilical vein endothelial cells (HUVECs). Endothelial SWELL1 (eSWELL1) expression positively regulates insulin and stretch-induced AKT2-eNOS signaling while negatively regulating mTOR signaling, HUVEC size, and migration, independent of VRAC current amplitude, via an eSWELL1-GRB2-Cav1-eNOS signaling complex. Endothelium-restricted SWELL1 KO (eSWELL1 KO) mice exhibit enhanced tube formation from ex-vivo aortic ring explants in matrigel angiogenesis assays, develop hypertension in response to chronic angiotensin II infusion and have impaired retinal blood flow with both diffuse and focal blood vessel narrowing in the setting of type 2 diabetes (T2D). These data demonstrate that SWELL1 antithetically regulates AKT-eNOS and mTOR signaling in endothelium and is required for maintaining vascular function, particularly in the setting of T2D.

SWELL1 is Highly Expressed and Functionally Encodes VRAC in Endothelium

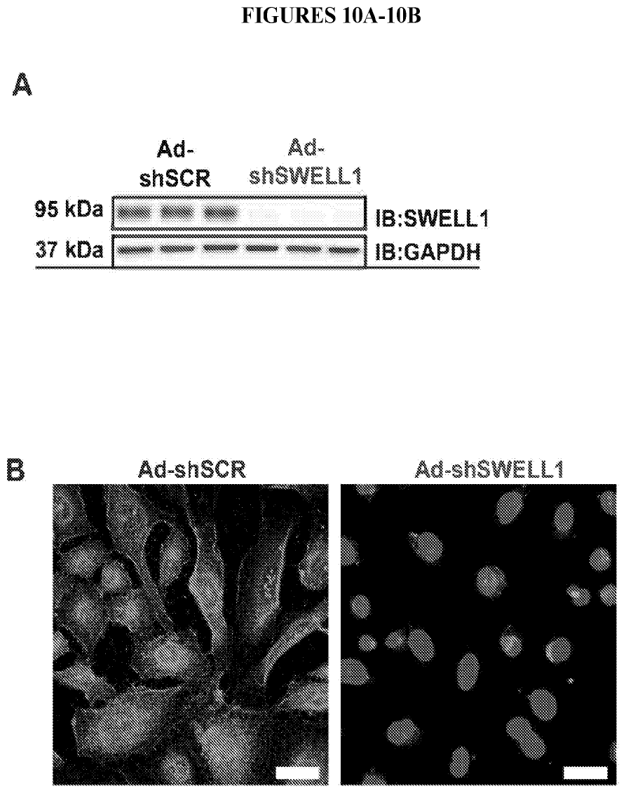

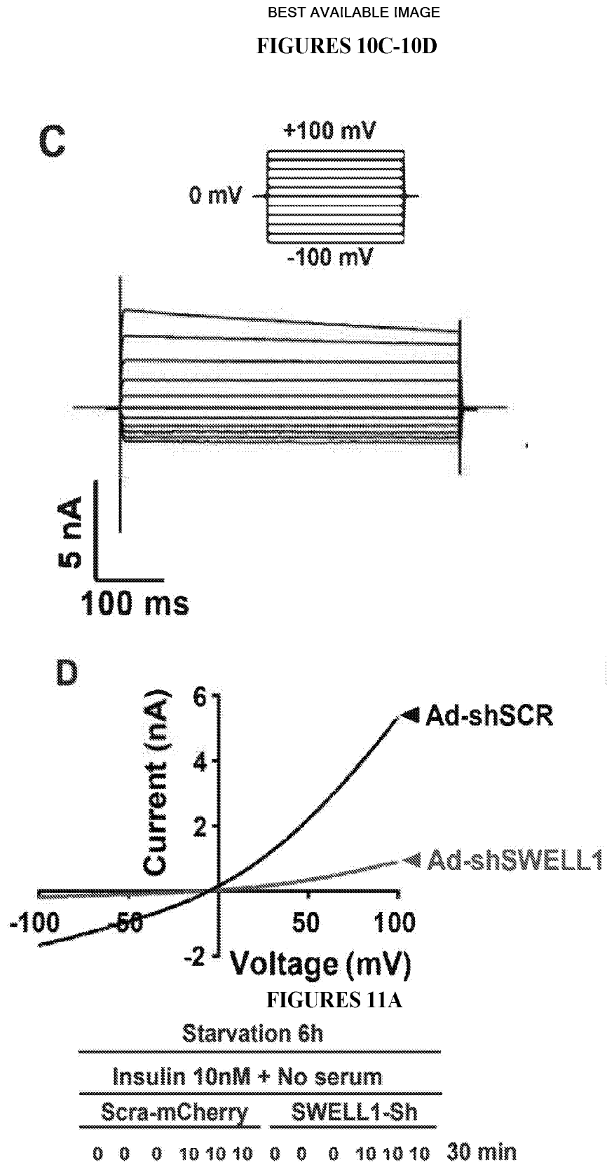

The volume-regulatory anion current (VRAC) has been measured and characterized in in endothelial cells for decades but the molecular identity of this endothelial ion channel remains elusive (Barakat, Int J Mol Med, 4, 323-332, 1999; Barakat et al., Circ Res, 85, 820-828, 1999; Nilius et al., Physiol Rev, 81, 1415-1459, 2001). To determine if the leucine-rich repeat containing membrane protein SWELL1 (LRRC8a) recently identified in cell lines (Qiu et al., Cell, 157, 447-458, 2014; Voss et al., Science, 344, 634-638, 2014) is required for VRAC in endothelial cells, as it is in adipocytes (Zhang et al., Nat Cell Biol, 19, 504-517, 2017), pancreatic β-cells (Kang et al., Nat Commun, 9, 367, 2018; Stuhlmann et al., Nat Commun, 9, 1974, 2018), nodose neurons (Wang et al., JCI Insight, 2, e90632, 2017) and spermatozoa (Luck et al., J Biol Chem, 293, 11796-11808, 2018), we first confirmed robust SWELL1 expression by Western blot (FIG. 10A) and immunostaining (FIG. 10B) in human umbilical vein endothelial cells (HUVECs). This SWELL1 expression is substantially reduced upon adenoviral transduction with a short-hairpin RNA directed to SWELL1 (Ad-shSWELL1-mCherry) as compared to a scrambled control (Ad-shSCR-mCherry). Next, we measured hypotonically-induced (210 mOsm) endothelial VRAC currents in HUVECs. These classic outwardly rectifying VRAC currents are prominent in HUVECs and significantly suppressed upon shSWELL1-mediated SWELL1 knock-down (FIG. 10C-E), consistent with SWELL1 functionally encoding endothelial VRAC.

SWELL1 Regulates Insulin-PI3K-AKT-eNOS, ERK and mTOR Signaling in Endothelium

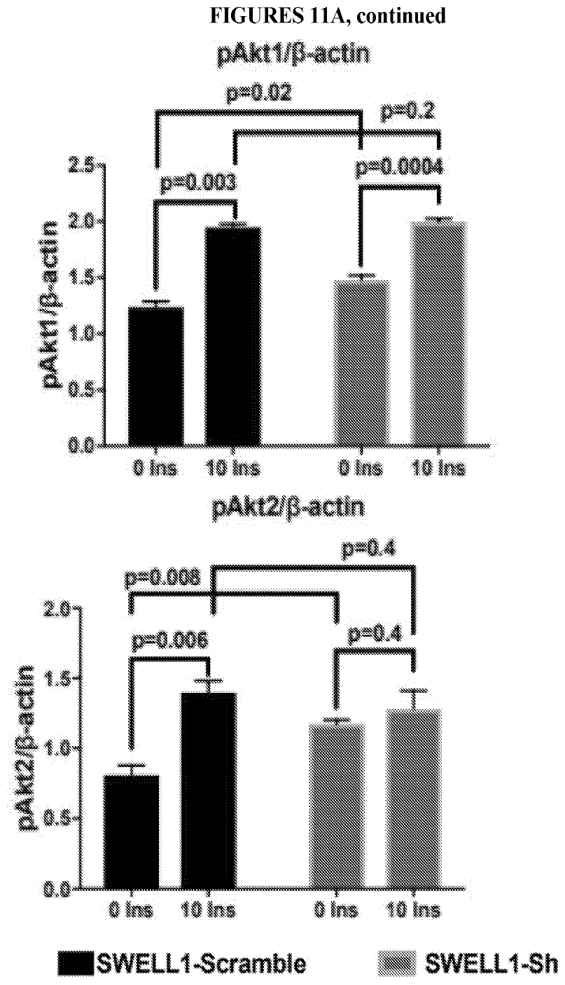

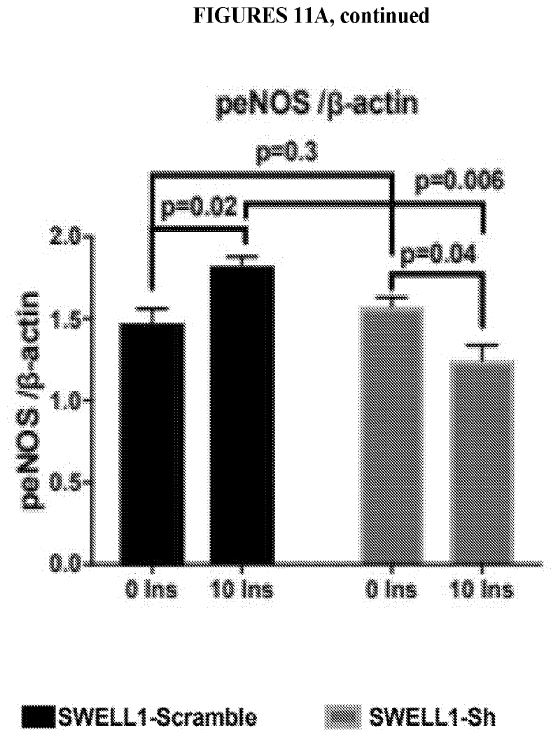

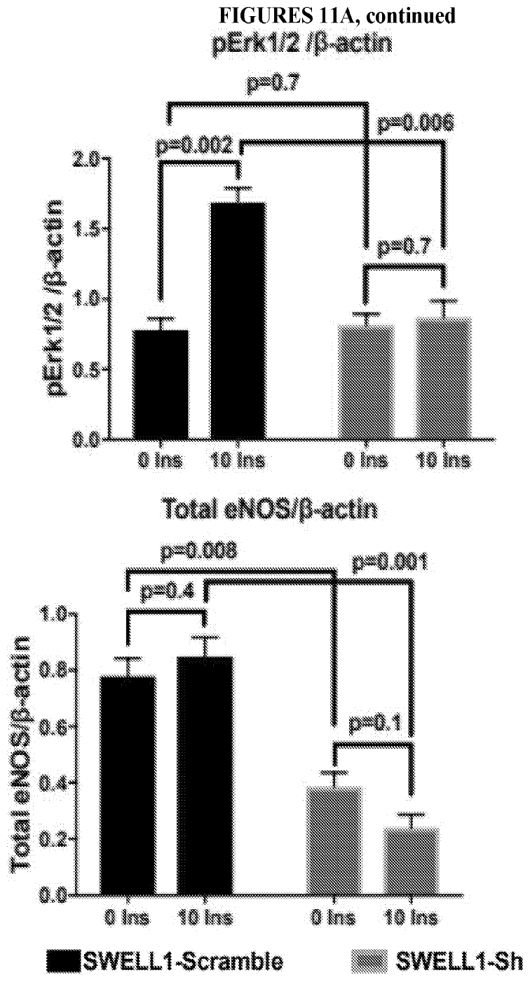

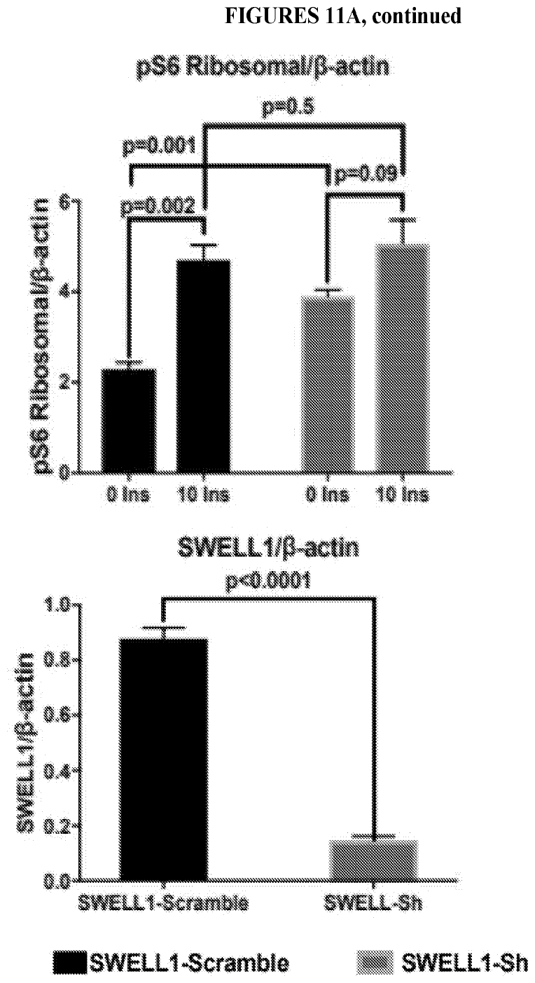

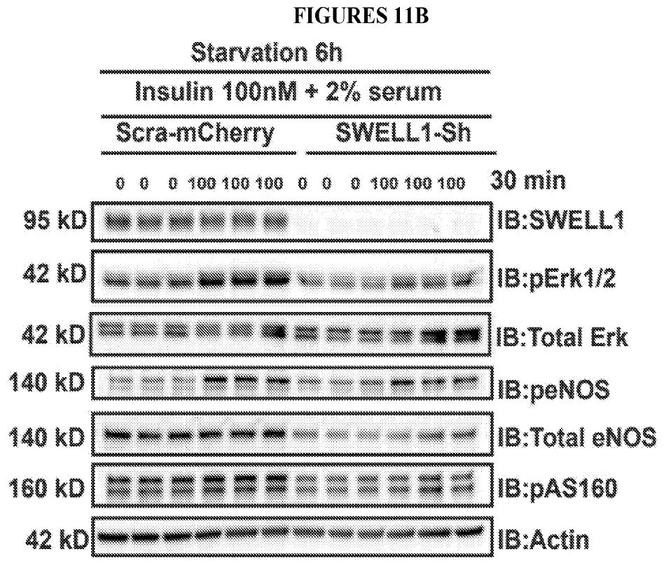

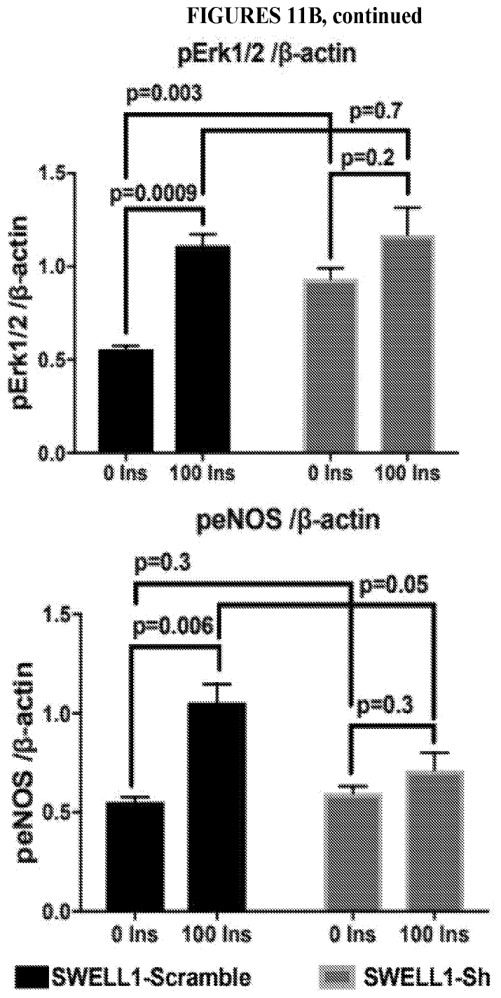

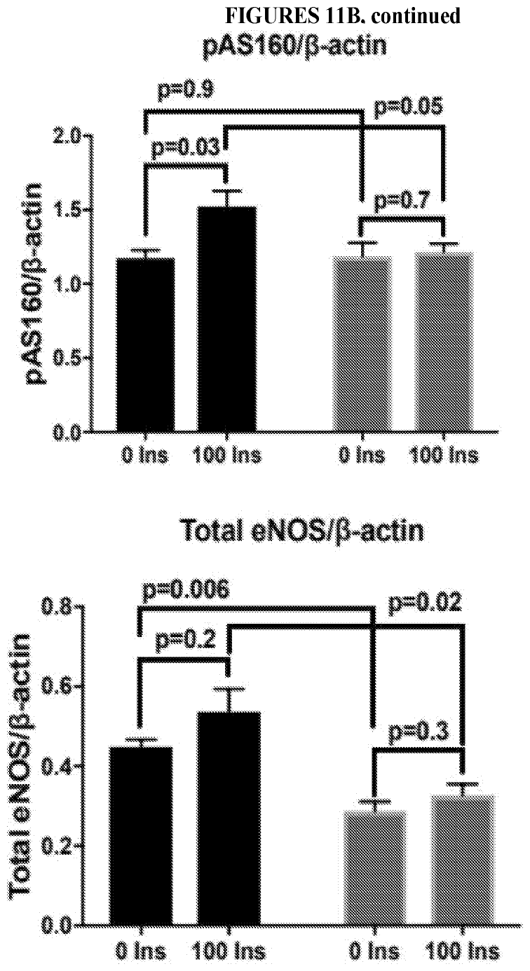

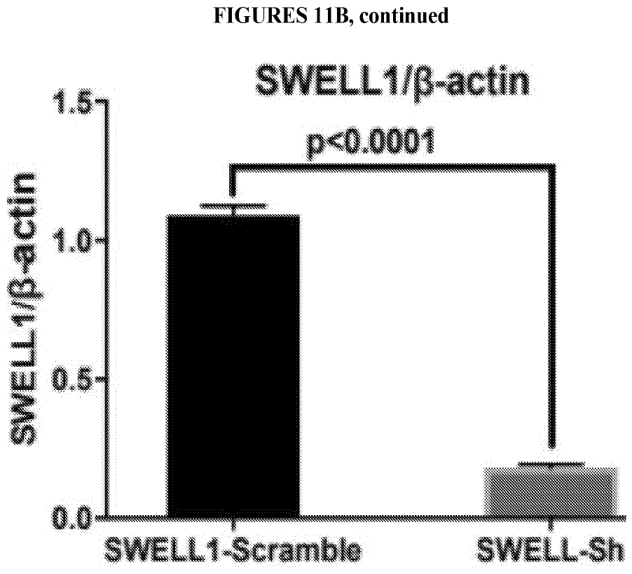

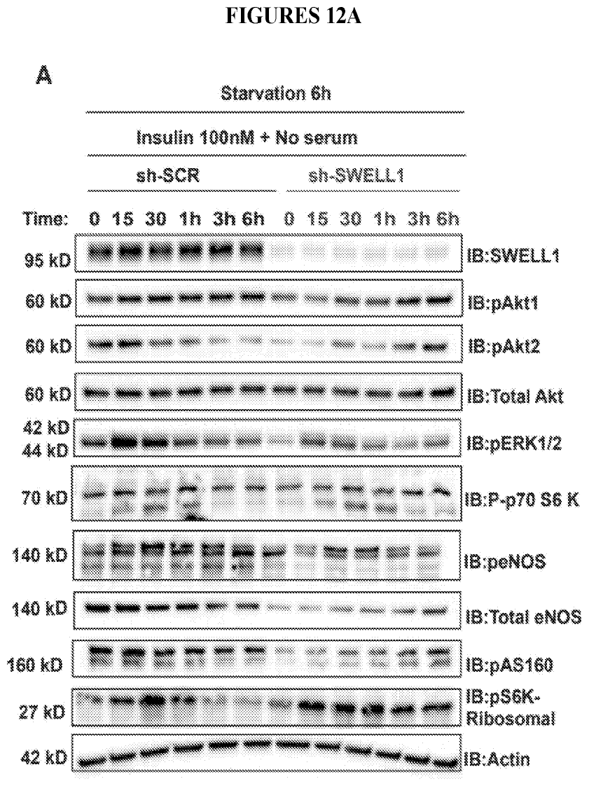

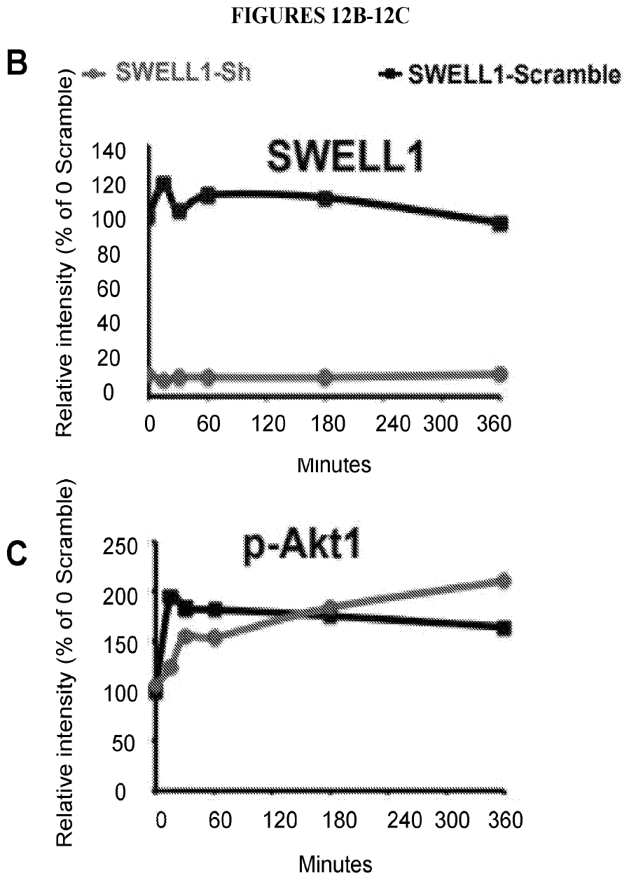

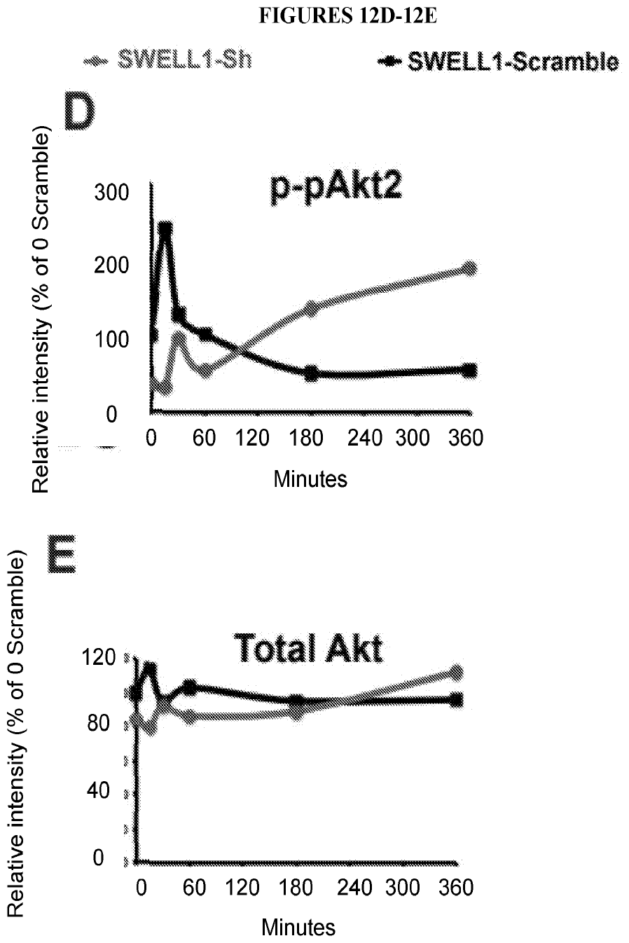

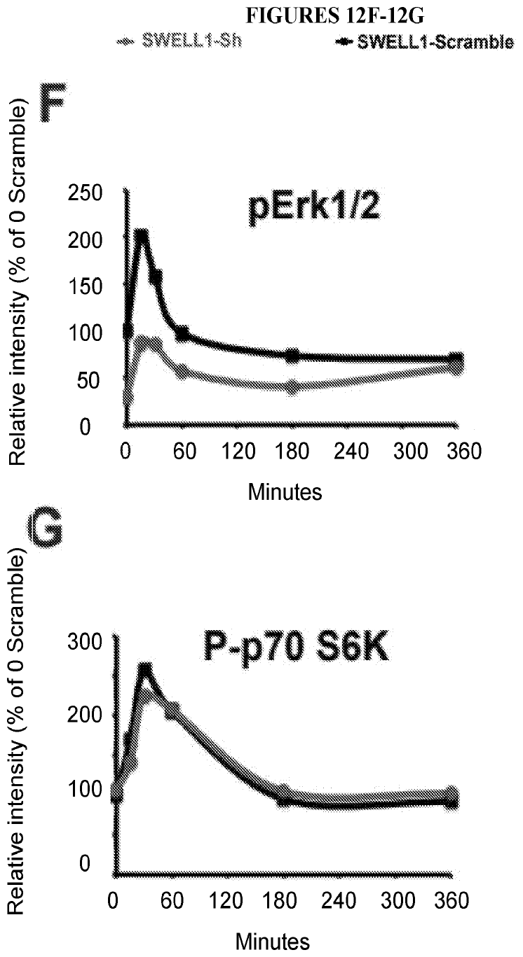

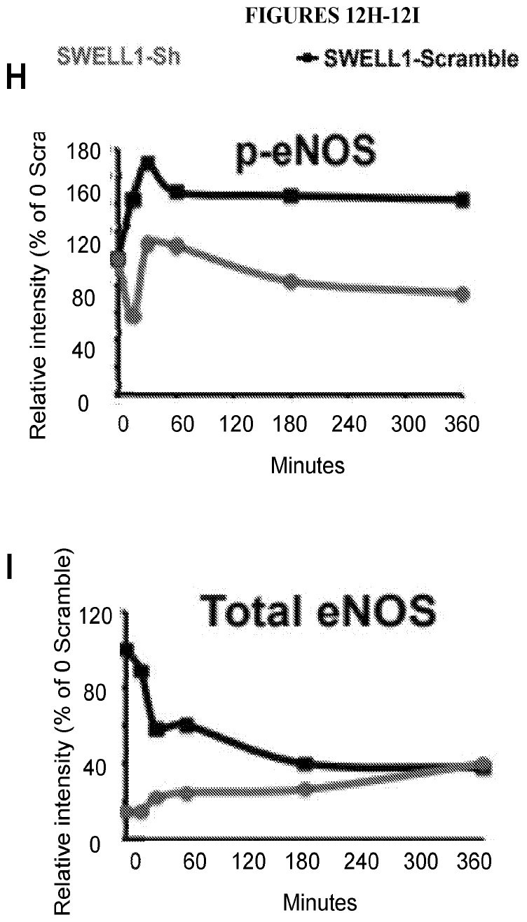

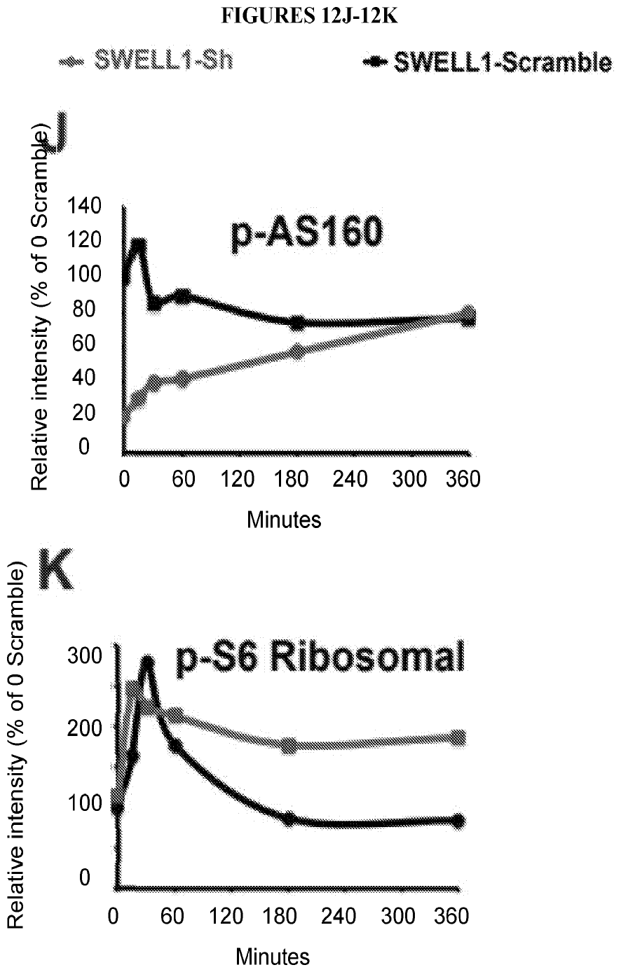

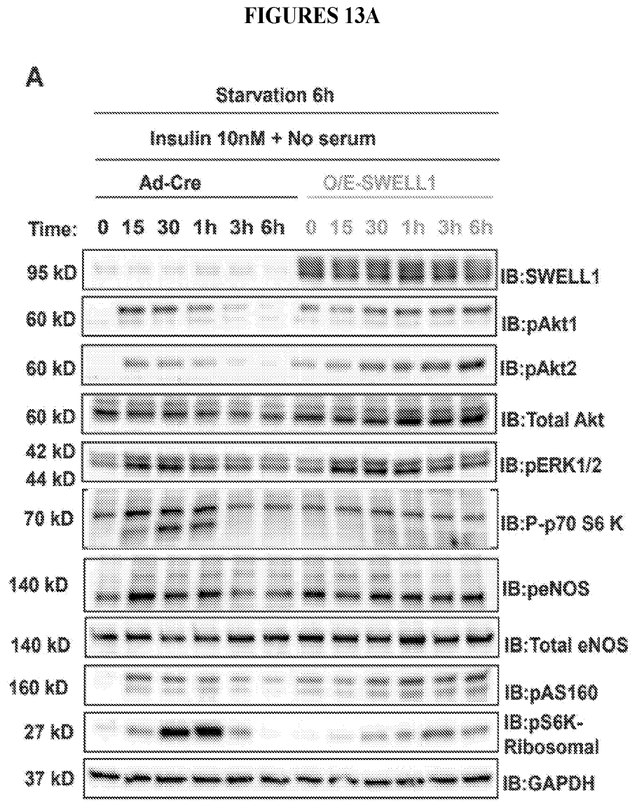

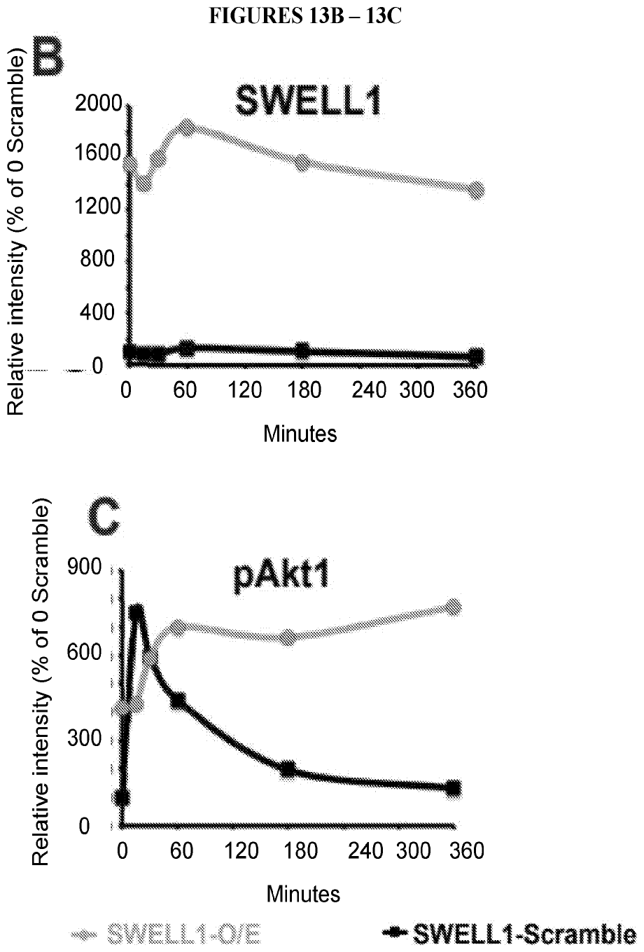

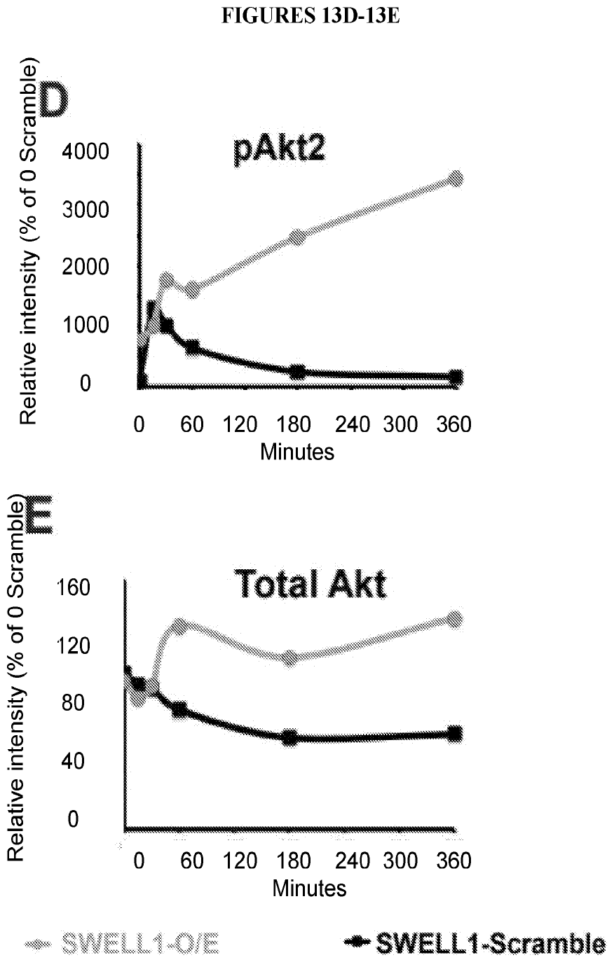

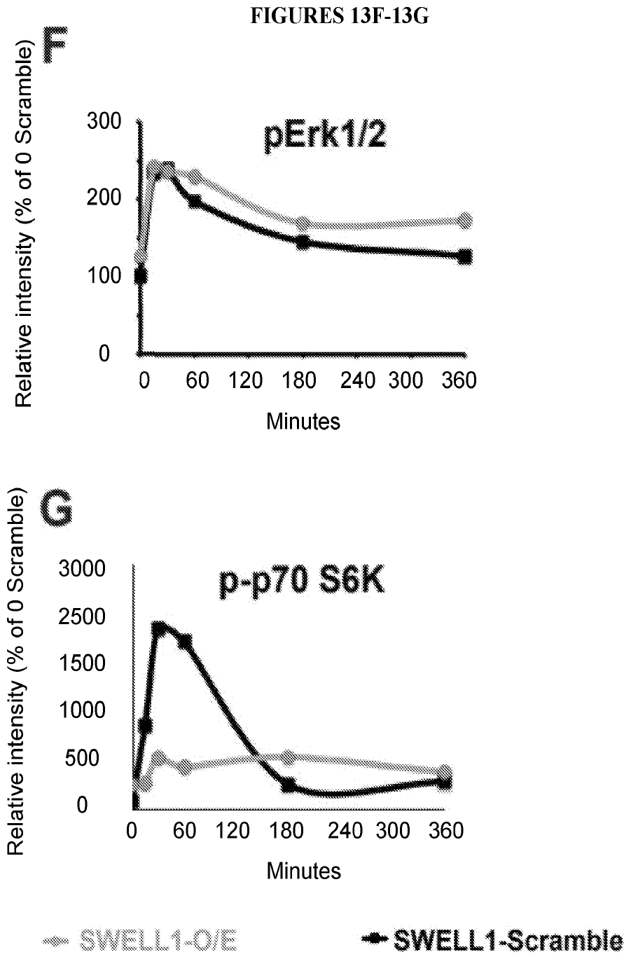

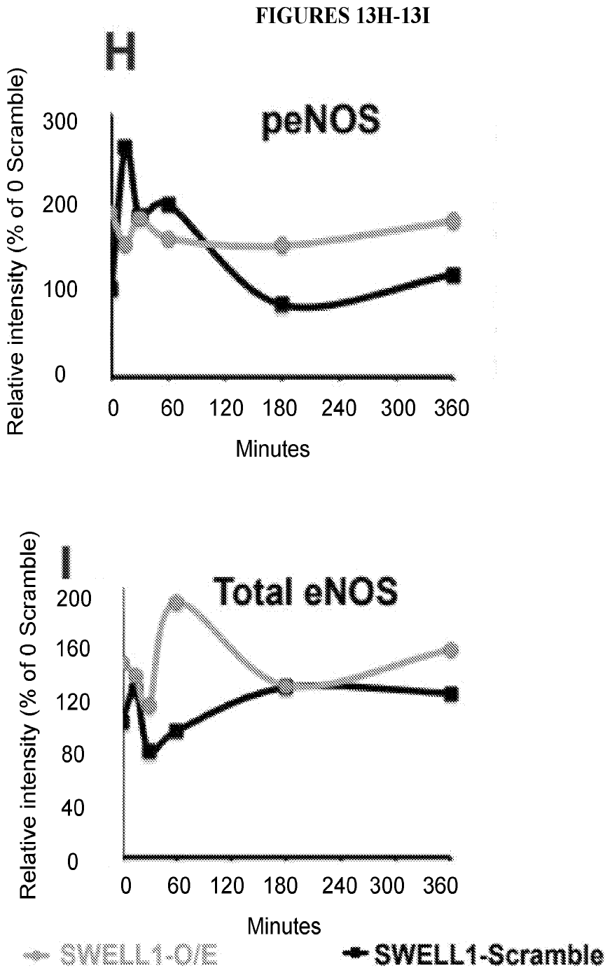

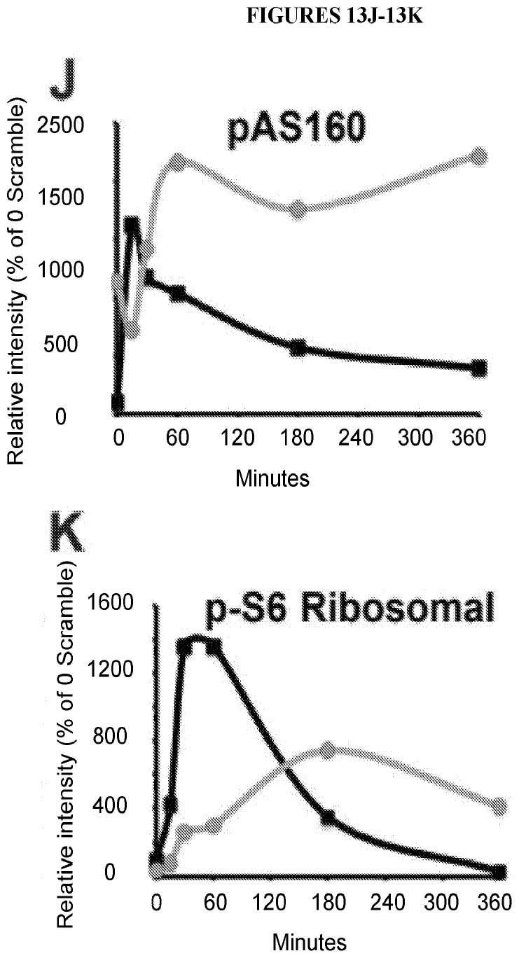

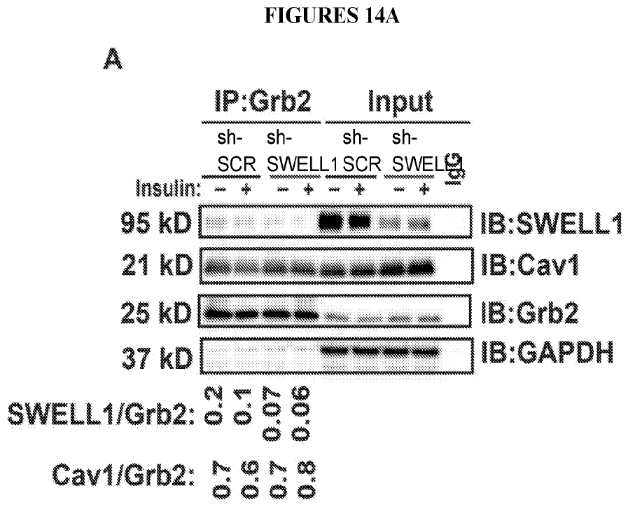

Our previous studies in adipocytes demonstrate that SWELL1 regulates insulin-PI3K-AKT signaling, adipocyte expansion and systemic glycemia, whereby SWELL1 loss-of-function induces an insulin-resistant pre-diabetic state (Xie et al., Channels (Austin), 11(6): 673-677, 2017; Zhang et al., Nat Cell Biol, 19, 504-517, 2017). Insulin signaling is also important in regulating endothelium and vascular function (Duncan et al., Diabetes, 57, 3307-3314, 2008; Kearney et al., Exp Physiol, 93, 158-163, 2008; Muniyappa et al., Rev Endocr Metab Disord, 14, 5-12, 2013). Moreover, insulin-resistance in Type 2 diabetes (T2D) is considered a systemic disorder and insulin-resistant endothelium is postulated to underlie impaired vascular function in T2D (Kearney et al., Exp Physiol, 93, 158-163, 2008; Muniyappa et al., Rev Endocr Metab Disord, 14, 5-12, 2013). As SWELL1 is highly expressed in endothelium (FIG. 10), and PI3K-AKT-eNOS signaling critical for endothelium-dependent vascular function (Morello et al., Cardiovasc Res, 82, 261-271, 2009), we next examined insulin-stimulated AKT-eNOS, ERK1/2 and mTOR signaling in SWELL1 KD compared to control HUVECs. Insulin-stimulated (10 and 100 nM) increases in pAKT2, pAS160, pERK and peNOS are significantly abrogated in HUVECs upon SWELL1 KD (FIGS. 11A&B), indicating that SWELL1 is necessary for insulin-PI3K, ERK signaling in endothelium, functioning as a positive regulator of insulin-signaling-analogous to adipocytes (Zhang et al., Nat Cell Biol, 19, 504-517, 2017). Curiously, insulin-stimulated pS6 ribosomal (mTOR signaling) is augmented in SWELL1 KD HUVECs compared to control, suggesting SWELL1 to be a negative regulator of mTOR in endothelium (FIG. 11A). Time-course experiments over 6 hours of insulin-stimulation reveal that these effects are also time-dependent (FIG. 12) but follow the same trends. To complement these SWELL1 loss-of-function experiments we performed SWELL1 gain-of-function experiments and examined these same signaling pathways. Overexpressing SWELL1 above endogenous SWELL1 protein levels is sufficient to augment insulin-stimulated pAKT, eNOS, p-eNOS and pERK (MAP kinase signaling) in HUVECs, while reducing p-p70, pS6 ribosomal protein (mTOR signaling, FIG. 13). To determine whether these changes in signaling upon SWELL1 overexpression are associated with increases in SWELL1-mediated VRAC we measured VRAC in Ad-shSCR, Ad-shSWELL1 and Ad-SWELL1 transduced HUVECs. Similar to previous reports in common cell lines (Qiu et al., Cell, 157, 447-458, 2014; Voss et al., Science, 344, 634-638, 2014), while SWELL1 loss-of-function reduces VRAC, SWELL1 overexpression in HUVECs does not increase VRAC current above basal WT levels. These data indicate that SWELL1 is both necessary and sufficient for insulin-AKT, eNOS, ERK and mTOR signaling in endothelium, and that this signaling is independent of SWELL1-mediated plasma membrane VRAC activity.

SWELL1 Interacts with GRB2, Cav1 and eNOS and Mediates Stretch-Dependent eNOS Signaling

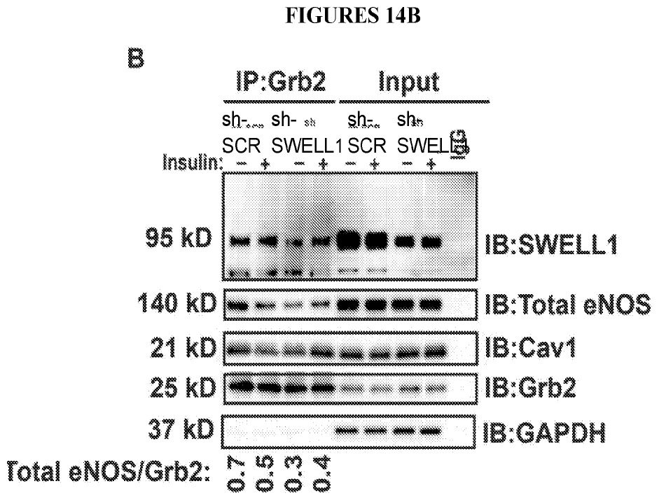

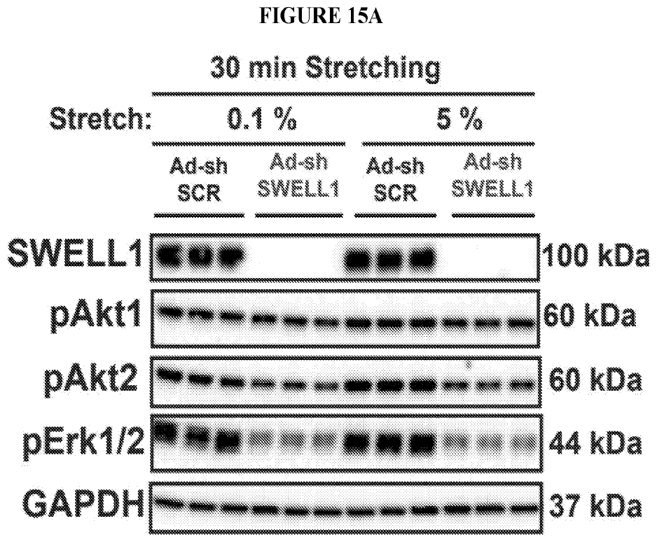

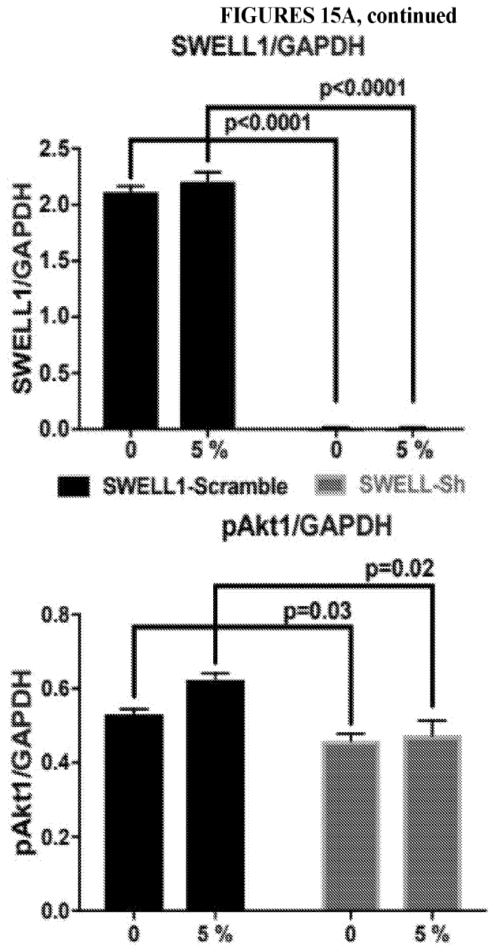

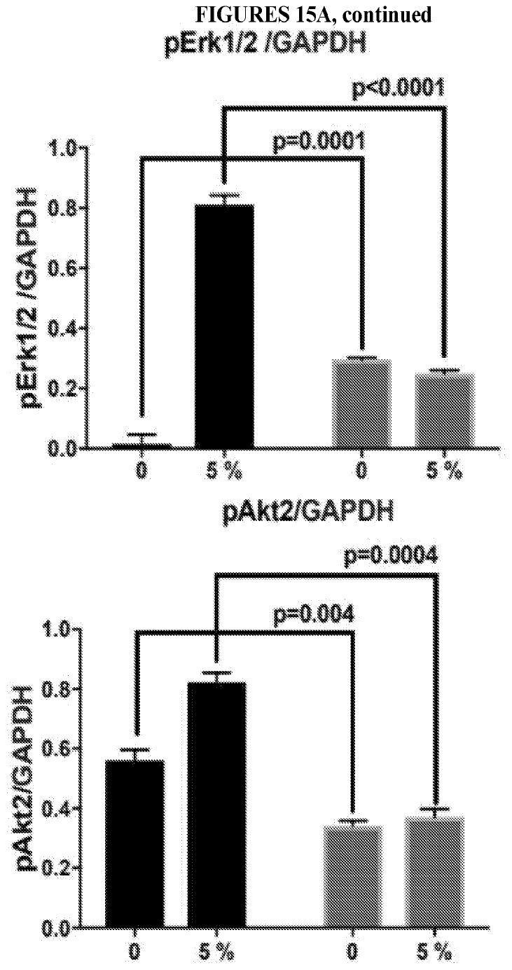

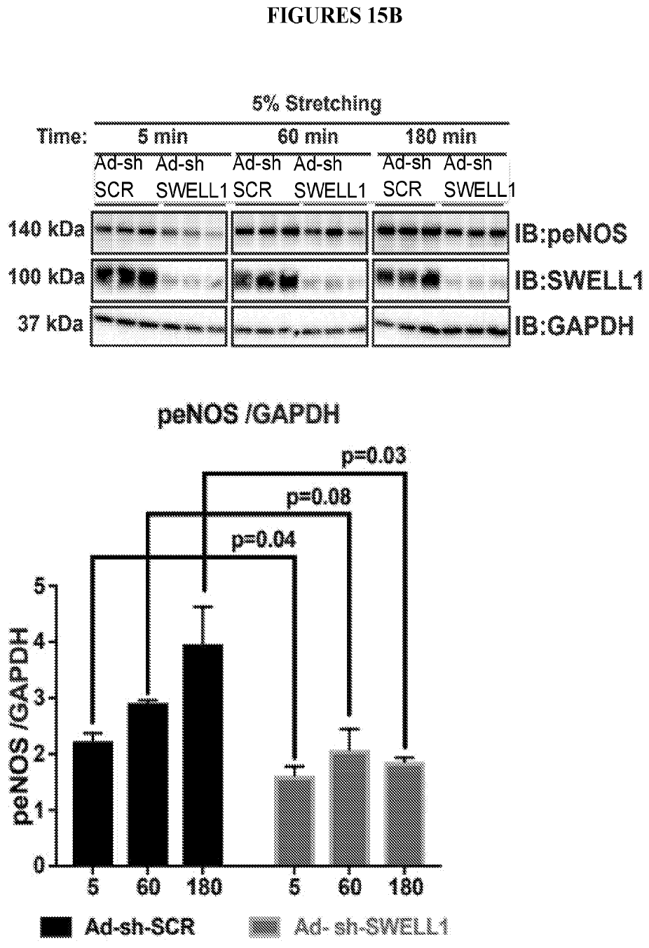

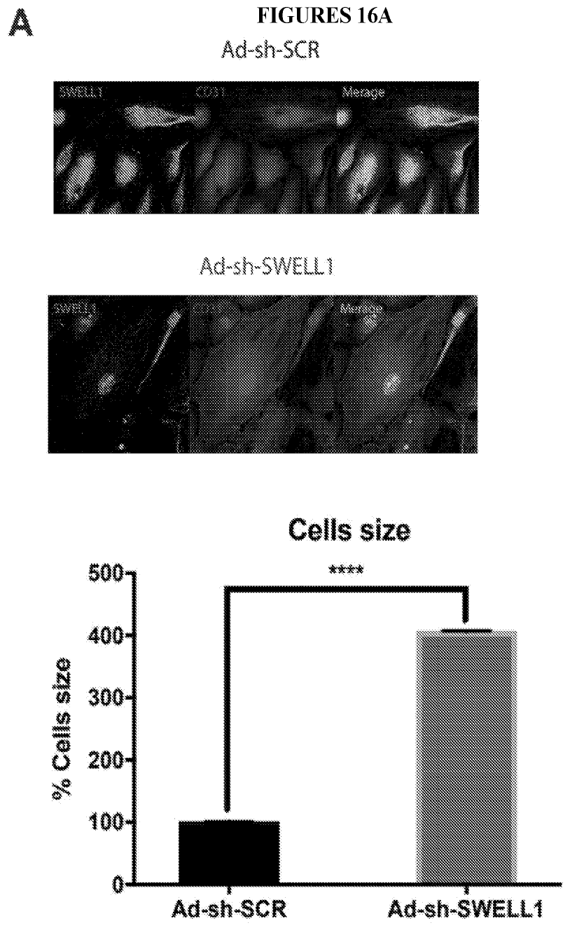

In adipocytes, the mechanism of SWELL1-mediated regulation of PI3K-Akt signaling involves SWELL1/GRB2/Cav molecular interactions. To determine if SWELL1 resides in a similar macromolecular signaling complex in endothelium we immunoprecipitated (IP) endogenous GRB2 from HUVECs. Upon GRB2 IP, we detect SWELL1 protein in shSCR treated HUVECs and less IP SWELL1 upon shSWELL1-mediated SWELL1 KD, consistent with a SWELL1-GRB interaction (FIGS. 14A&B), in addition to both Cav1 (FIGS. 14A&B) and eNOS (FIG. 14B). These data suggest that endothelial SWELL1 resides in a signaling complex that includes GRB2, Cav1 and eNOS, consistent with the findings that GRB2 and Cav1 interact, and that Cav1 regulates eNOS via a direct interaction (Ju et al., J Biol Chem, 272, 18522-18525, 1997; Venema et al., Biochem Biophys Res Commun, 236, 155-161, 1997; Goligorsky et al., Am J Physiol Renal Physiol, 283, F1-10, 2002). Moreover, these data are also in-line with the notion that caveoli form mechanosensitive microdomains (Sedding et al., Circ Res, 96, 635-642, 2005; Sinha et al., Cell, 144, 402-413, 2011; Nassoy et al., Trends Cell Biol, 22, 381-389, 2012) that regulate VRAC (Trouet et al., J Physiol, 520 Pt 1, 113-119, 1999; Trouet et al., Biochem Biophys Res Commun, 284, 461-465, 2001) and that VRAC can be activated by mechanical stimuli in a number of cell types, including endothelium (Barakat, Int J Mol Med, 4, 323-332, 1999; Nakao et al., Am J Physiol, 276, C238-249, 1999; Nilius et al., Physiol Rev, 81, 1415-1459, 2001; Romanenko et al., Am J Physiol Cell Physiol, 282, C708-718, 2002; Browe et al., J Gen Physiol, 122, 689-702, 2003; Browe et al., J Gen Physiol, 127, 237-251, 2006). Given that the endothelial cells respond to stretch stimuli to regulate vascular tone via activation of eNOS, we next examined the SWELL1-dependence of stretch-AKT, ERK1/2 and eNOS signaling in HUVECs (FIG. 15). Similar to insulin-stimulation, 5% stretch is sufficient to stimulate AKT and ERK1/2 signaling in HUVECs and this is abrogated in SWELL1 KD HUVECS, indicating that SWELL1 also regulates stretch-dependent AKT and ERK1/2 signaling in HUVECs (FIG. 15A). Similarly, we observe abrogation of time-dependent p-eNOS signaling with 5% stretch in SWELL1 KD HUVECS compared to control (FIG. 15B). Taken together, these data position SWELL1 as a regulator of both insulin and stretch-mediated PI3K-AKT-eNOS signaling in endothelium via a SWELL1-GRB2-Cav1-eNOS macromolecular complex.

SWELL1 Regulates Endothelial Cell Size, Migration and Sprouting Angiogenesis

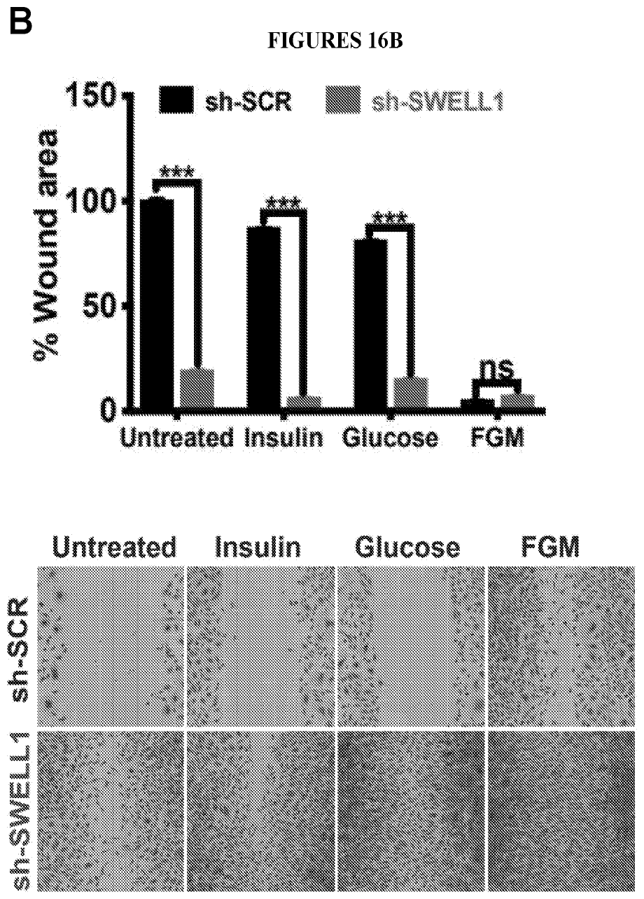

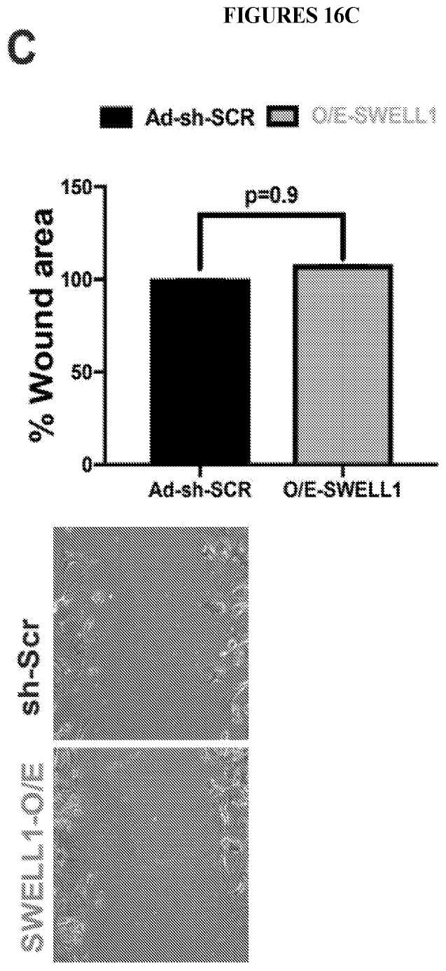

Given that SWELL1 regulates mTOR signaling in endothelium (FIG. 11) and that mTOR is a known regulator of cell size and migration, we next assessed SWELL1 dependent HUVEC size and migration. Consistent with insulin-stimulated induction of mTOR signaling in SWELL1 KD HUVECs we find that SWELL1 KD HUVECs are significantly 4-fold larger (FIG. 16A), and migrate >5-fold faster in scratch assays (FIG. 16B) compared to controls. Conversely, SWELL1 overexpression significantly slows migration rate to control levels (FIG. 16C), as expected with SWELL1-mediated reductions in mTOR signaling (FIG. 13).

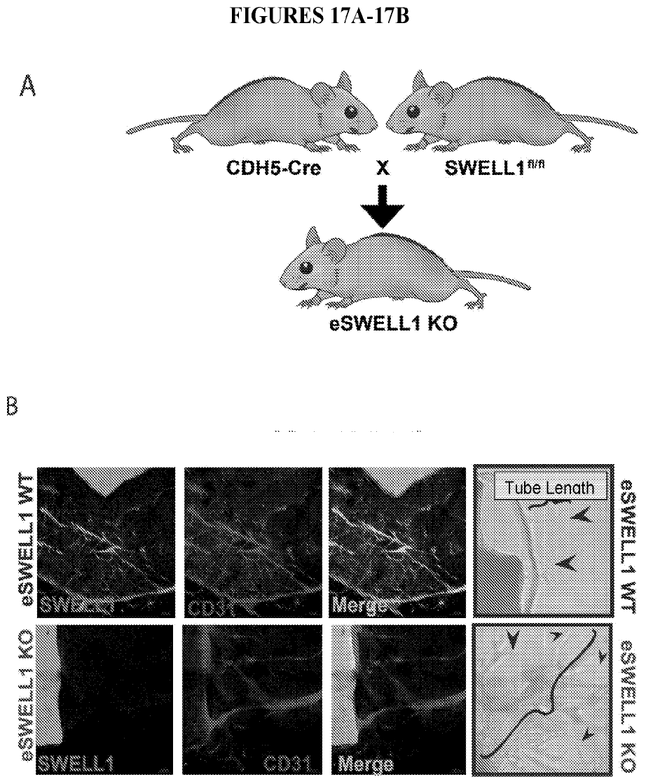

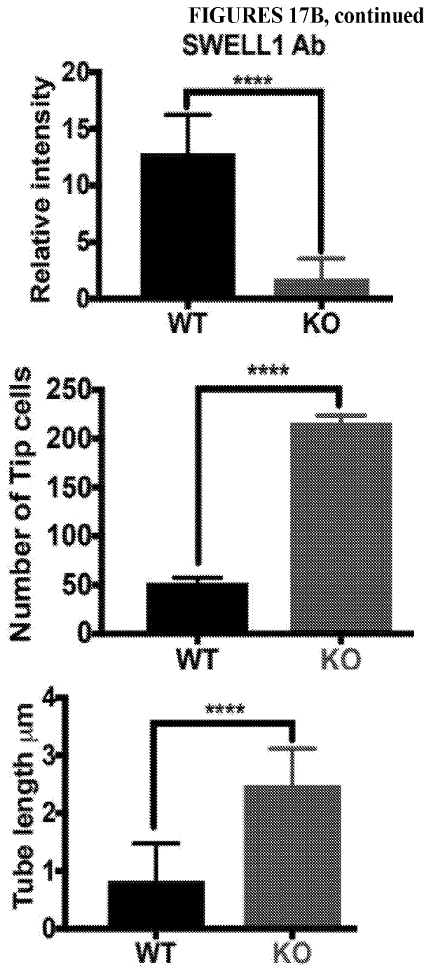

To examine the functional consequences of endothelial SWELL1 ablation in vivo we generated endothelial-targeted SWELL1 KO mice (eSWELL1 KO) by crossing SWELL1 floxed mice with the endothelium-restricted CDH5-Cre mouse (FIG. 17A). Immunofluorescence staining of aortic ring explants revealed ablation of SWELL1 from CD31+ primary endothelial cells sprouting from the explants (FIG. 17B). Consistent the SWELL1-mediated regulation of HUVEC migration observed in scratch assays (FIG. 16B), ex-vivo sprouting angiogenesis is also significantly enhanced in upon SWELL1 ablation, based on both tube length and number of tip cells in eSWELL1 KO mice as compared to SWELL1 floxed mice (FIG. 17B). Taken together, these data suggest that SWELL1 may regulate angiogenesis via mTOR signaling.

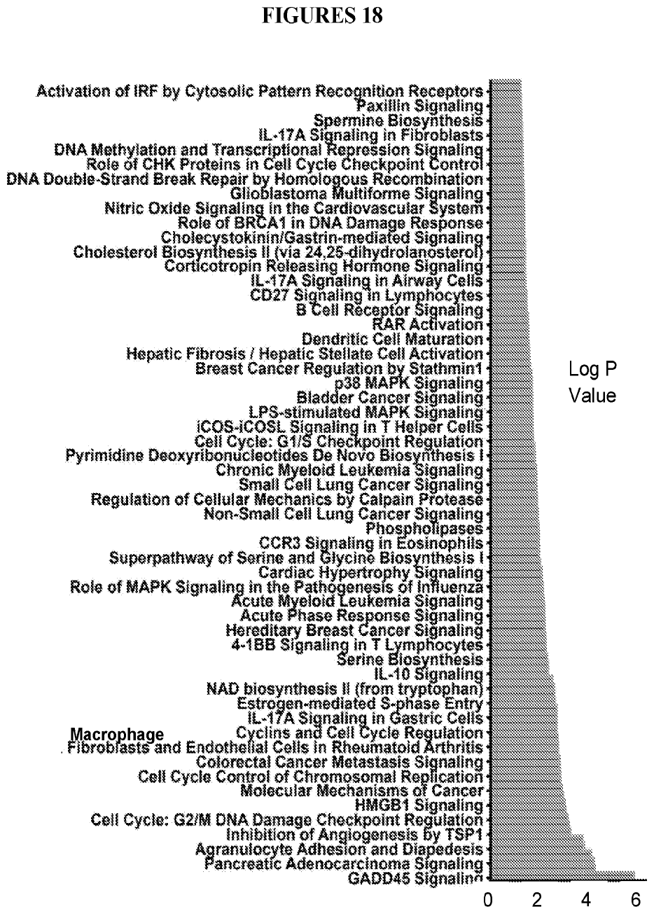

Indeed, genome-wide transcriptome analysis of SWELL1 KD HUVEC compared to control (RNA sequencing) reveal multiple pathways enriched regulating angiogenesis, migration and tumorigenesis, including GADD45, IL-8, p70S6K, TREM1, angiopoeitin and HGF signaling (FIG. 18). In addition, pathways linked to cell adhesion and renin-angiotensin signaling are enriched—pathways altered in vasculature in the setting of atherosclerosis and Type 2 diabetes (T2D).

eSWELL1 KO Mice Exhibit Mild Angiotensin-II Stimulated Hypertension and Impaired Retinal Blood Flow in the Setting of Type 2 Diabetes

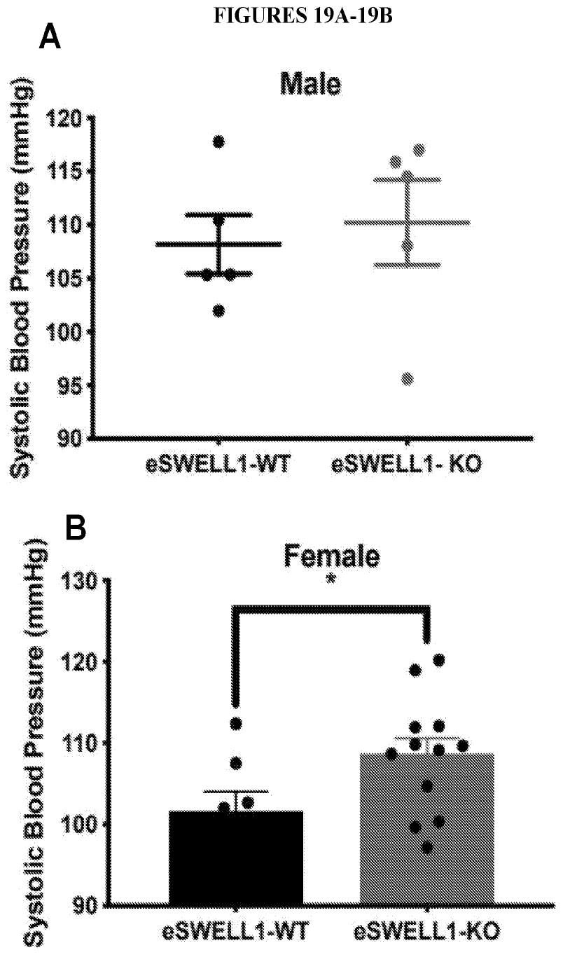

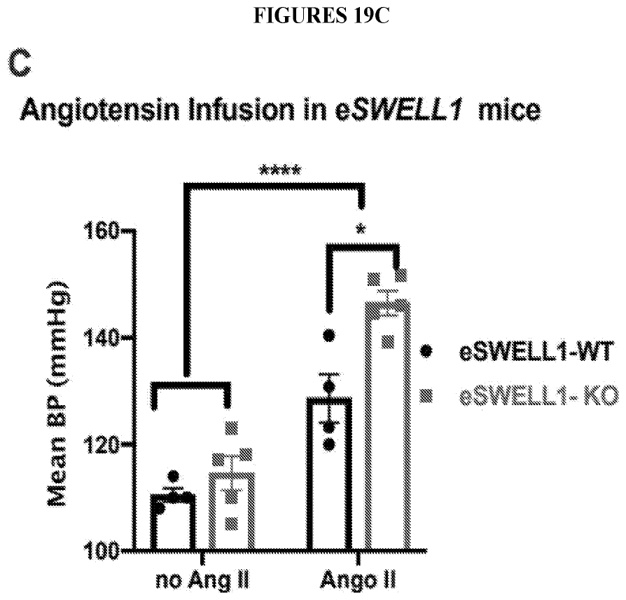

Based on our findings that SWELL1 regulates AKT-eNOS signaling in endothelium, and that eNOS signaling is central to systemic arterial and/or pulmonary arterial blood pressure regulation, we next examined systemic arterial blood pressures in eSWELL1 KO mice compared to WT controls (SWELL1 fl/fl mice). Male mice exhibit no significant differences in systolic systemic arterial blood pressure under basal conditions (FIG. 19A), while female mice are mildly hypertensive relative to WT mice (FIG. 19B). However, after 4 weeks of angiotensin-II infusion (Ang II), male eSWELL1 KO mice develop exacerbated systolic hypertension as compared to AngII-treated WT mice (FIG. 19C). These data are consistent with endothelial dysfunction and impaired vascular relaxation in eSWELL1 KO mice, resulting in a propensity for systolic hypertension.

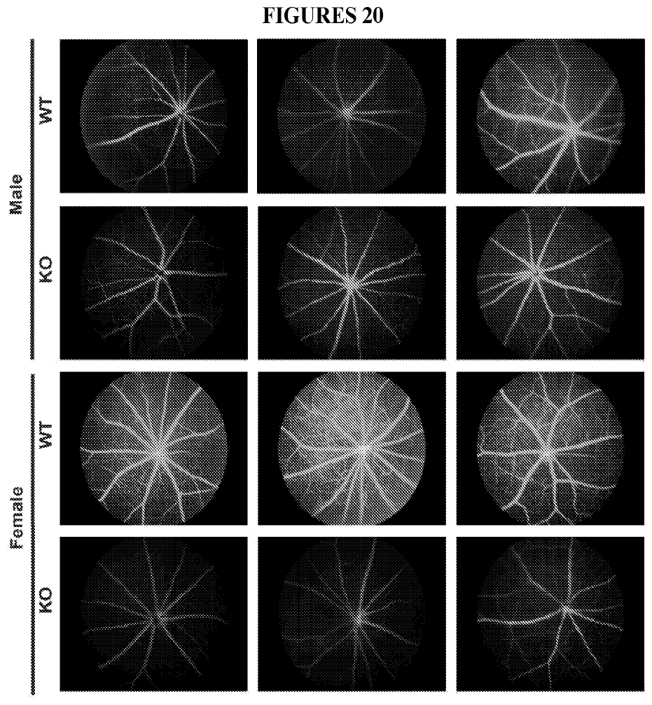

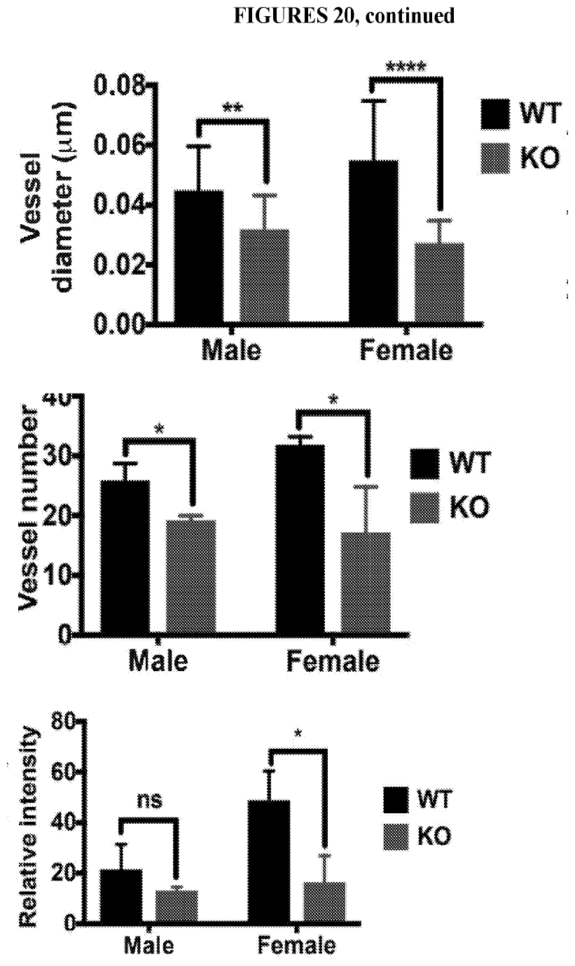

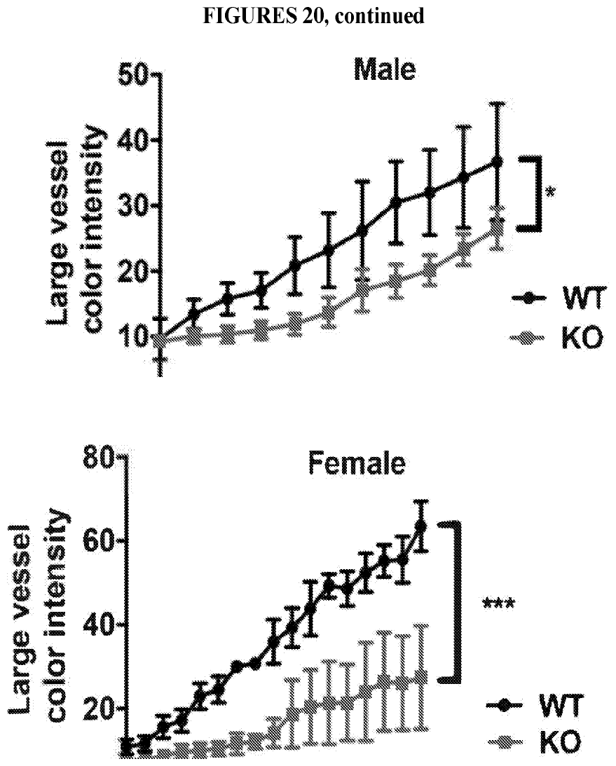

As endothelial dysfunction may also result in impaired blood flow we performed retinal imaging during i.p. injection of fluorescein to quantify retinal vessel blood flow and morphology in WT and eSWELL1 KO mice raised on either a regular diet and high-fat high-sucrose (HFHS) diet. Mice raised on a regular diet have mild impairments in retinal blood flow, based on the relative rate of rise of the fluorescein signal in the retinal arteries. There is also mild thinning of retinal vessels in eSWELL1 KO as compared to WT mice, principally in female mice. In mice raised on HFHS diet, retinal blood flow is more severely impaired with significant focal, and diffuse retinal vessel narrowing in eSWELL1 KO mice compared to WT mice (FIG. 20), and this is markedly worse in female compared to male mice.

Taken together, our findings reveal that SWELL1 is highly expressed in endothelium and functionally encodes endothelial VRAC. SWELL1 tunes insulin and stretch-stimulated AKT-eNOS, and mTOR signaling in endothelium and resides in a SWELL1-GRB2-Cav1-eNOS signaling complex to regulates endothelial cell migration, angiogenesis and vascular reactivity in vivo, especially the setting of T2D.

All documents cited herein are incorporated by reference. While certain embodiments of invention are described, and many details have been set forth for purposes of illustration, certain of the details can be varied without departing from the basic principles of the invention.

The use of the terms “a” and “an” and “the” and similar terms in the context of describing embodiments of invention are to be construed to cover both the singular and the plural, unless otherwise indicated herein or clearly contradicted by context. The terms “comprising,” “having,” “including,” and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. In addition to the order detailed herein, the methods described herein can be performed in any suitable order unless otherwise indicated herein or otherwise clearly contradicted by context. The use of any and all examples, or exemplary language (e.g., “such as”) provided herein, is intended merely to better illuminate embodiments of invention and does not necessarily impose a limitation on the scope of the invention unless otherwise specifically recited in the claims. No language in the specification should be construed as indicating that any non-claimed element is essential to the practice of the invention.

What is claimed is:

1. A method for preventing and/or treating nonalcoholic fatty liver disease (NAFLD) in a patient in need of such therapy, comprising administering a therapeutically effective amount of a SWELL1 modulator to the patient.

2. The use of a SWELL1 modulator for preventing and/or treating nonalcoholic fatty liver disease (NAFLD).

3. A method for regulating vascular tone, systemic arterial and/or pulmonary arterial blood pressure and/or blood flow in a patient in need of such treatment, comprising administering to the patient a therapeutically effective amount of a SWELL1 modulator to the patient.

4. The method of claim 3, wherein the method comprises administering to the patient a therapeutically effective amount of a SWELL1 modulator to the patient so as to regulate vascular tone.

5. The method of claim 3, wherein the method comprises administering to the patient a therapeutically effective amount of a SWELL1 modulator to the patient so as to regulate systemic arterial and/or pulmonary arterial blood pressure.

6. The method of claim 3, wherein the method comprises administering to the patient a therapeutically effective amount of a SWELL1 modulator to the patient so as to regulate blood flow.

7. The use of a SWELL1 modulator for regulating vascular tone, systemic arterial and/or pulmonary arterial blood pressure and/or blood flow.

8. A method for preventing and/or treating agammaglobulinemia or other immune deficiency in a patient in need of such therapy, comprising administering a therapeutically effective amount of a SWELL1 modulator to the patient.

9. The use of a SWELL1 modulator for preventing and/or treating agammaglobulinemia or other immune deficiency.

10. A method for preventing and/or treating male infertility in a patient in need of such therapy, comprising administering a therapeutically effective amount of a SWELL1 modulator to the patient.

11. The use of a SWELL1 modulator for preventing and/or treating male infertility.

12. The method or use of any one of claims 1-11, wherein the SWELL1 modulator is DCPIB, clomiphene, nafoxidine or tamoxifen.

13. The method or use of any one of claims 1-11, wherein the SWELL1 modulator is DCPIB.

14. The method or use of any one of claims 1-11, wherein the SWELL1 modulator is a compound of formula I, or a salt thereof 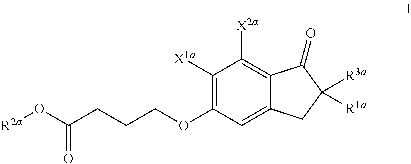
wherein:
X^{1a} and X^{2a} are independently halo;
R^{1a} is C₁₋₆ alkyl, 3-6 membered cycloalkyl, or phenyl, wherein the C₁₋₆ alkyl is optionally substituted with 3-6 membered cycloalkyl;
R^{2a} is hydrogen or C₁₋₆ alkyl, wherein the C₁₋₆ alkyl is optionally substituted with carboxy; and
R^{3a} is C₁₋₆ alkyl.

15. The method or use of any one of claims 1-11, wherein the SWELL1 modulator is a compound of formula II, or a salt thereof 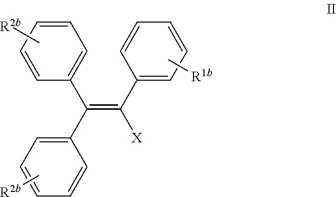
wherein:
R^{1b} is hydrogen, halo or methoxy;
only one of R^{2b} is —O(CH₂)ₙ—NR^{3b}R^{4b};
the other R^{2b} is hydrogen, halo or methoxy;
each of R^{3b} and R^{4b} is independently H or C₁₋₆ alkyl, or R^{3b} and R^{4b} together with the nitrogen to which they are attached form aziridino, azetidino, morpholino, piperazino, pyrrolidino or piperidino;
n is an integer from 2 to 4; and
X is halo.

16. The method or use of any one of claims 1-11, wherein the SWELL1 modulator is a compound of formula III, or a salt thereof 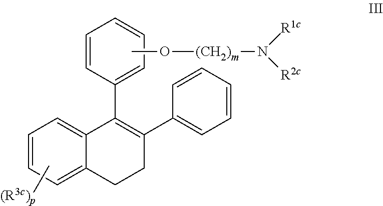
wherein:
each of R^{1c} and R^{2c} is independently H or C₁₋₈ alkyl, or R^{1c} and R^{2c} together with the nitrogen to which they are attached form aziridino, azetidino, morpholino, piperazino, pyrrolidino or piperidino; wherein the aziridino, azetidino, morpholino, piperazino, pyrrolidino and piperidino are optionally substituted with one or more C₁₋₆ alkyl;
m is an integer from 2 to 6,
R^{3c} is C₁₋₈ alkoxy; and
p is an integer from 1 to 4.

17. The method or use of any one of claims 1-11, wherein the SWELL1 modulator is a compound of formula IV, or a salt thereof 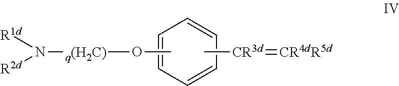
wherein:
each of R^{1d} and R^{2d} is independently H or C₁₋₆ alkyl, or R^{1d} and R^{2d} together with the nitrogen to which they are attached form aziridino, azetidino, morpholino, piperazino, pyrrolidino or piperidino;
each of R^{3d} and R^{4d} is independently aryl which is optional substituted with one or more groups selected from C₁₋₆ alkyl, C₁₋₆ alkoxy, C₁₋₆ dialkylamino, or halo;
R^{5d} is C₁₋₆ alkyl or C₁₋₆ alkenyl, wherein the C₁₋₆ alkyl is optionally substituted with aryl; and
q is an integer from 2 to 6.

18. The method or use of any one of claims 1-17, wherein the administration or use of the SWELL1 modulator and/or SWELL1-LRRC8 binding molecule is sufficient to upregulate the expression of SWELL1 and/or stability and/or assembly of SWELL1-LRRC8 complexes and/or membrane trafficking and/or SWELL1-LRRC8 signaling, or alter expression and/or associated of a SWELL1 associated protein (e.g., LRRC8b,c,d,e, GRB2, Cav1, IRS1, or IRS2).

19. The method or use of any one of claims 1-18, wherein the administration or use of the SWELL1 modulator is sufficient to upregulate the expression of SWELL1.
